(19) United States
(12) Patent Application Publication
(10) Pub. No.: US 2020/0206227 A1
(43) Pub. Date: Jul. 2, 2020

(54) TYROSINE KINASE INHIBITOR FORMULATIONS FOR THE TREATMENT OF MAST CELL-MEDIATED INFLAMMATORY DISEASES AND METHODS OF USE THEREOF

(71) Applicant: The Board of Trustees of the Leland Stanford Junior University, Stanford, CA (US)

(72) Inventors: William H. Robinson, Palo Alto, CA (US); Anna Postolova, Emerald Hills, CA (US); Harini Raghu, Mountain View, CA (US)

(21) Appl. No.: 16/672,086

(22) Filed: Nov. 1, 2019

Related U.S. Application Data
(63) Continuation of application No. 15/582,263, filed on Apr. 28, 2017, now abandoned.
(60) Provisional application No. 62/329,032, filed on Apr. 28, 2016.

(51) Int. Cl. A61K 31/519; A61K 31/506; A61K 9/00; A61K 9/16

(52) U.S. Cl. CPC A61K 31/519; A61K 31/506; A61K 9/0019; A61K 9/0048; A61K 9/0043; A61K 9/0073; A61K 9/1647

(57) ABSTRACT

Methods of treating mast cell-mediated inflammatory diseases are provided by local administration a therapeutically effective amount of a tyrosine kinase inhibitor to a patient in need thereof.

### CROSS REFERENCE

This application claims benefit and is a Continuation of U.S. application Ser. No. 15/582,263, filed Apr. 28, 2017, which claims benefit of U.S. Provisional Patent Application No. 62/329,032, filed Apr. 28, 2016, which applications are incorporated herein by reference in their entirety.

### FEDERALLY SPONSORED RESEARCH AND DEVELOPMENT

This invention was made with Government support under contract AR063676 awarded by the National Institutes of Health. The Government has certain rights in the invention.

### FIELD OF INVENTION

This invention relates to methods and pharmaceutical compositions for treating mast cell-mediated inflammatory diseases with tyrosine kinase inhibitors (TKI) to treat, to slow, and to arrest or reverse the discomfort and structural damage to tissues caused by such mast cell-mediated inflammatory diseases. Mast cell-mediated inflammatory diseases include, but are not limited to, osteoarthritis, crystal-induced arthritis, psoriatic arthritis, tenosynovitis, synovitis, allergic/non-allergic/chronic rhinitis, rhinosinusitis, conjunctivitis and ocular allergies, uveitis, nasal polyps, asthma, aspirin exacerbated respiratory disease (AERD), chronic obstructive pulmonary disease (COPD), and eosinophilic esophagitis. More specifically, formulations of specific TKIs, including but not limited to inhibitors of KIT, SRC, SYK and JAK that target development, activation and function of mast cells and other immune cells like macrophages, are administered locally, for example as a sustained release dosage form injected into a joint or delivered intranasally for allergies or nasal inflammation, swallowed for local esophageal action, or applied topically to the eye, with or without an immediate release component, that results in efficacy.

### BACKGROUND OF THE INVENTION

Mast cells are generally long-lived, tissue-dwelling immune cells critically involved in allergic and anaphylactic reactions. It is known in the art that mast cell activation through cross-linking of their surface receptors for IgE (FceRI) results in rapid degranulation and release of vasoactive, pro-inflammatory and nociceptive mediators that include histamine, cytokines and proteolytic enzymes. Owing to this, several current therapies that are in use for diseases involving mast cell degranulation are primarily targeted at inhibiting or antagonizing or blocking these mast cell mediators from performing their function (e.g., anti-histamines) for immediate relief of symptoms. The invention disclosed herein relates to methods of treating diseases that involve aberrant mast cell activation by targeting mast cell development and/or activation i.e., methods of targeting molecules upstream of mast cell mediators such as histamine. Specifically, this invention relates to targeting mast cells via inhibiting or blocking tyrosine kinase signaling pathways mediating mast cell development and/or survival and/or migration and/or activation and/or degranulation using long-acting, sustained-release formulations of various tyrosine kinase inhibitors.

Tyrosine kinases involved in mast cell and/or macrophage activation include but are not limited to those that belong to KIT, SRC, SYK and JAK families of kinases for which various tyrosine kinase inhibitors have been developed. We discovered a hitherto unknown critical and direct pathogenic role for mast cells in osteoarthritis using mice that possess a functional mutation in the crucial receptor tyrosine kinase for mast cell development, Kit, were protected against the development of osteoarthritis. Further, we unexpectedly discovered that tyrosine kinase inhibitors that target KIT or SRC or JAK protect against the development of mouse osteoarthritis by not only inhibiting mast cell activation but also by inhibiting mast cell development and by inhibiting mast cell survival.

The inventors are unaware of any small molecule TKIs given by intraarticular administration, let alone intraarticular injection of TKI in the form of long acting, controlled and sustained release particle formulations containing TKIs for the therapy of OA and crystal-induced arthritis. The inventors are unaware of any particle formulations containing small molecule TKIs given by intraarticular, intranasal, intraocular, intraauricular, swallowed, or inhaled administration for local targeting, let alone administration of TKI in the form of long acting, controlled and sustained release particle formulations comprising TKIs to resolve tissue inflammation, to prevent, slow, halt, or reverse tissue damage, to prevent, slow, halt, or reverse symptoms related to mast cell-mediated inflammatory conditions.

Described herein the term “mast cell-mediated inflammatory diseases” broadly refers to inflammatory and/or allergic diseases/conditions wherein mast cells participate in the induction and/or propagation and/or maintenance of inflammation, through selective release of mediators. The term mast cell-mediated inflammatory diseases also refers to conditions wherein mast cells can be activated to degranulate rapidly, not only by IgE and antigen signaling via the high-affinity receptor for IgE (FceRI), but also by a diverse group of stimuli including signaling from tyrosine kinases. In addition, the term mast cell-mediated inflammatory diseases refers to conditions where mast cells contribute to the symptomatology of said diseases but also critically modulate inflammatory pathways involved in initiation, propagation, tissue remodeling or tissue damage of said diseases. The term mast cell-mediated inflammatory diseases also refers to chronic diseases or conditions that involve aberrant mast cell development or mast cell survival or mast cell activation or mast cell degranulation.

Examples of mast cell-mediated inflammatory diseases include but are not limited to rheumatoid arthritis (RA), psoriatic arthritis (PsA), reactive arthritis, gouty arthritis or gout, pseudogout arthritis or CPPD arthritis; uveitis and allergic/non-allergic/chronic rhinitis, rhinosinusitis, conjunctivitis and ocular allergies, nasal polyps, asthma, aspirin exacerbated respiratory disease, COPD, and eosinophilic esophagitis. The clinical manifestations and biological mechanisms of these conditions differ significantly, but it has been discovered that mast cells play a critical role in the pathobiology of these diseases and conditions.

With regards to osteoarthritis (OA), a number of studies had shown that mast cells, and several mast cell mediators are present in the synovium and synovial fluid of individuals with osteoarthritis. However, whether mast cells and/or their mediators play a direct pathogenic role in osteoarthritis was unknown until our novel and unexpected findings showed a direct pathogenic role for mast cells in OA. Herein, OA is also designated as a mast cell-mediated inflammatory disease.

Receptor tyrosine kinases (RTKs) and cytoplasmic tyrosine kinases or non-RTKs are among the signaling molecules that are most crucial for innate immune responses mediated by mast cells and macrophages (examples provided in Table 1). Tyrosine kinases are a subfamily of protein kinases that play a critical role in cell signaling and are involved in a variety of mast cell-mediated inflammatory disorders including cell proliferation, survival, angiogenesis and metastasis. Tyrosine kinase inhibitors (TKIs) have revolutionized the treatment of certain forms of cancers, raising hopes for many patients with otherwise unresponsive tumors. Studies have also shown effectiveness of systemic administration for the treatment of RA (Genovese M C et al. Baricitinib in patients with refractory rheumatoid arthritis (2016) NEJM, 374(13), 1243-1252. PMID: 27028914), pulmonary fibrosis, and PSA. As such, several TKIs have been approved for use in the treatment of cancer and inflammatory diseases (examples provided in Table 2).

Imatinib (also referred to as imatinib mesylate or imat) is a small-molecule tyrosine kinase inhibitor that targets breakpoint cluster region-Abelson kinase (Bcr-Abl), and also inhibits a narrow spectrum of additional protein tyrosine kinases including stem cell factor receptor (KIT), SRC, platelet-derived growth factor receptor (PDGFR), colony stimulating factor-1 receptor (CSF-1R; FMS), and is used to treat chronic myelogenous leukemia (CML).

Nilotinib has been developed as a new more potent and selective inhibitor of Bcr-Abl. These drugs also inhibit a narrow spectrum of additional protein tyrosine kinases, including Abl, lymphocyte-specific protein tyrosine kinase (LCK), KIT, PDGFR, discoidin domain receptor (DDR), and CSF-1R kinases.

Dasatinib (also referred to as dasa) is a potent adenosine triphosphate and competitive inhibitor of tyrosine kinases. Dasatinib, previously known as BMS-354825, is a cancer drug produced by Bristol-Myers Squibb and sold under the trade name Sprycel. Dasatinib is an oral Bcr-Abl tyrosine kinase inhibitor (inhibits the “Philadelphia chromosome”) and SRC family tyrosine kinase inhibitor approved for first line use in patients with CML and Philadelphia chromosome-positive acute lymphoblastic leukemia (Ph+ ALL). In addition to Bcr-Abl and Abl, dasatinib also inhibits the tyrosine kinases KIT, platelet-derived growth factor receptor (PDGFR), Eph receptors (EPHR), RC and BTK family members.

Tofacitinib (formerly tasocitinib, CP-690,550, also referred to as tofa) is an orally available tyrosine kinase inhibitor, the first member of a novel class of medications, the JAK inhibitors. It inhibits phosphorylation of the tyrosine kinases JAK1 and JAK3, and thereby blocks IL-6R-mediated phosphorylation of STAT1 and STAT3, and STAT5. However, it is currently categorized as a pan-JAK inhibitor preferentially inhibiting JAK1 and JAK3 and, to a lesser extent, JAK2 with minimum effect on TYK2. In November 2012, the U.S. FDA approved tofacitinib to treat adults with moderately to severely active rheumatoid arthritis who have had an inadequate response to, or who are intolerant of, methotrexate. Additional tyrosine kinase inhibitors that inhibit JAK1, JAK2 and/or JAK3 including ruxolitinib, ABT494, baricitinib, CYT387, filgotinib, lestaurtinib, pacritinib, JSI-124 and CHZ868.

The non-receptor spleen tyrosine kinase SYK is involved in signal transduction in a variety of cell types. In particular, it is a key mediator of immune receptors signaling in host inflammatory cells (B cells, mast cells, macrophages and neutrophils), important for both allergic and antibody-mediated autoimmune diseases. Dysregulated SYK kinase activity also allows growth factor-independent proliferation and transforms bone marrow-derived pre-B cells that are able to induce leukemia. Examples of SYK inhibitors in development include fostamatinib (R788) (Ruzza P et al. (2009) Therapeutic prospect of Syk inhibitors. Expert opinion on therapeutic patents, 19(10), 1361-1376. DOI: 10.1517/13543770903207039). Additional tyrosine kinase inhibitors that inhibit SYK including entospletinib (GS-9973) and R406 (the active metabolite fostamatinib).

There exists a need for an improved pharmaceutical composition that can provide a quick onset of action as well as a long lasting effect; have physical characteristics that facilitate local administration into various parts of the body; and be shelf-stable. In particular, a stable, long-acting pharmaceutical composition suited for local admiration including but not limited to intraarticular injection, intralesional injection, intraocular application, intraocular injection, intranasal delivery, intraauricular delivery, inhaled delivery, and swallowed administration, such as those disclosed in this invention is desirable.

### SUMMARY OF THE INVENTION

We describe herein our discovery that mast cells and macrophages activated via tyrosine kinase signaling pathways play a crucial role in the pathogenesis of inflammatory joint diseases. Mast cells and macrophage also play a key role in the pathogenesis of allergic diseases. Mast cells and macrophages activated through a tyrosine kinase(s) produce a large variety of pathogenic mediators including inflammatory cytokines/chemokines and tissue degradative enzymes. Unexpectedly, it was discovered tyrosine kinase inhibitors, for example, imatinib or dasatinib or tofacitinib, attenuated not only mast cell and macrophage activation but also the development and maturation of mast cells and macrophages during these diseases, for example, during osteoarthritis (OA) and during crystal-induced arthritis.

Described herein are compositions, methods and systems for reducing pain, and/or inflammation and/or tissue damage associated with mast cell-mediated inflammatory conditions using tyrosine kinase inhibitors (TKIs). In most embodiments, the compositions described herein use TKIs, alone and not inhibitors of other kinases. In some embodiments, however, the compositions of the present invention can include other kinase inhibitors. Other kinase inhibitors include inhibitors that target serine or threonine kinases, including those that inhibit MAPK (mitogen-activated protein kinases). The described compositions offer a broad yet unique set of inhibitors of cell surface receptor tyrosine kinases (e.g., KIT) and non-receptor tyrosine kinases (e.g., JAK, SYK, SRC) which were discovered to play critical roles in the pathogenesis of mast cell-mediated inflammatory joint diseases such as osteoarthritis and crystal-induced arthritis. The invention builds on our novel and unexpected discovery that long-term administration of such TKIs inhibit not only activation of immune cells including mast cells and macrophages but also their development and maturation and in some cases, migration to the site of inflammation.

The present invention describes novel pharmaceutical compositions for local administration and sustained release of TKIs from biocompatible, biodegradable, polymeric nanoparticles and/or biocompatible, biodegradable, polymeric particle formulations. The invention describes methods comprising administration to a target site in a subject in need of treatment, an effective amount of a pharmaceutical composition comprising one or more TKIs, wherein one or more TKIs are administered by one or more controlled release nanoparticle or microparticle systems. In the practice of the invention, the administration is localized and sustained.

In some embodiments the invention described herein provides compositions and methods for the treatment of pain and inflammation mediated by mast cells using TKI/PLGA nanoparticle and/or microparticle formulations. The compositions and methods provided herein are TKIs in a PLGA nanoparticle and/or microparticle formulation. The TKI/PLGA nanoparticle and/or microparticle formulations provided herein are suitable for local administration via injection (such as intraarticular or intraocular) or topical application (such as intranasal and intraocular), swallowed to affect local structures and inhaled administration. Suitable TKIs for the present application include but are not limited to imatinib, dasatinib, tofacitinib, as well as other TKIs, including salts or esters thereof.

Any pharmaceutically acceptable biodegradable polymer known in the art can be used to provide TKI containing particles as described herein. Suitable biodegradable polymers include but are not limited poly-a-hydroxy acid esters such as polylactic acid (PLLA or DLPLA), polyglycolic acid, polylactic-co-glycolic acid (PLGA), polylactic acid-co-caprolactone; poly (ester-co-amide) copolymers; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide), polyanhydrides, polyphosphazenes, polyaminoacids etc. In particular embodiments, the biodegradable polymer is PLGA with molar compositions having a lactic acid (LA):glycolic acid (GA) ratio ranging from 100:0 to 50:50 molecular weight of 7 kDa-100 kDa. Additionally, two or more forms of the biocompatible, biodegradable PLGA can be employed, one being the more hydrophobic end-capped polymer with the terminal residues functionalized as esters, and the other being the more hydrophilic uncapped polymer with the terminal residues existing as carboxylic acids.

It is appreciated by one skilled in the art that the degradation rates of said PLGA particles and drug release from said particles can be influenced by different parameters: (i) the molecular weight: increasing the molecular weight of conventional PLGAs from 7 to 100 kDa, degradation rates were reported to range from several weeks to several months; (ii) the ratio of lactic acid (LA) to glycolic acid (GA):PLGA with a higher content of LA are less hydrophilic, absorb less water and subsequently degrade more slowly, as a consequence of the presence of methyl side groups in PLA making it more hydrophobic than PGA. An exception to this rule is the copolymer 50:50 which exhibits the faster degradation; (iii) stereochemistry: mixtures of D and L lactic acid monomers are most commonly used for PLGA fabrication, as the rate of water penetration is higher in amorphous D,L regions, leading to accelerated PLGA degradation; and (iv) end-group functionalization: polymers that are end-capped with esters (as opposed to the free carboxylic acid) demonstrate longer degradation half-lives. Moreover, the shape of the PLGA particle (e.g., particle size) strongly affects PLGA degradation behavior depending on the accessibility of water. In addition, acidic surrounding media accelerate PLGA degradation due to autocatalysis.

These TKI containing PLGA nanoparticles and/or microparticles and formulations thereof are collectively referred to herein as “TKI/PLGA particles” and “TKI/PLGA particle formulations,” where these terms are used interchangeably. “TKI/polymer particles” include “TKI/PLGA particles” as well as TKI particles formulated with other polymers. The target for the general composition of the TKI/PLGA particles described herein will generally range from 10 to 90% TKI in the composition, % of polylactic acid in the polylactic acid polyglycolic acid (PLGA) copolymer can be 0-100%, e.g., about 30% TKI, in 50/50 PLGA with molecular weight of 7-17 kDa, inherent viscosity 0.16-0.24 dL/g, and the average particle size of the nanoparticles is 20 nm-100 μm.

US2014031167 discloses anti-inflammatory agents that may be included in a pharmaceutical composition for administration into the intraarticular space of a joint in combination with triamcinolone acetonide (TCA) PLGA microparticles as a secondary agent in addition to the therapeutically active TCA/PLGA particle. However, there is no disclosure in US2014031167 of a pharmaceutical composition comprising anti-inflammatory agents alone in a sustained-release formulation, let alone of such a pharmaceutical composition comprising one or more TKIs, for example, imatinib, or a pharmaceutically acceptable salt thereof, at a therapeutically effective dose used as the active agent in a local and sustained release biodegradable nanoparticle or microparticle formulation administered by intraarticular injection or intranasal or intraocular route to a subject in need of treatment for medical conditions including inflammatory and allergic diseases such as inflammation of the joints (especially osteoarthritis and crystal-induced arthritis), nasal inflammatory and allergic conditions, and ocular inflammatory and allergic (especially uveitis and conjunctivitis).

Although US20090136579 discloses tyrosine kinase inhibitors that target platelet-derived growth factor receptors (PDGFRs) may be included in a pharmaceutical composition as a nanoparticle delivery system for intra-cellular delivery by means of local injection devices or systems such as stents, there is no disclosure in US 20090136579 of a pharmaceutical composition comprising one or more TKIs other than those that target PDGFRs, for example, tofacitinib, or a pharmaceutically acceptable salt thereof, in a local and sustained release biodegradable nanoparticle or microparticle formulation administered by intraarticular injection or intra-nasal or intra-ocular route to a subject in need of treatment for medical conditions including inflammatory and allergic diseases such as inflammation of the joints (especially osteoarthritis and crystal-induced arthritis), nasal inflammatory and allergic conditions, and ocular inflammatory and allergic (especially uveitis and conjunctivitis).

US20140148474 A1 discloses SYK tyrosine kinase inhibitors that may be potentially useful in treating diseases resulting from inappropriate activation of mast cells and related inflammatory and allergic responses. However, there is no disclosure in US 20140148474 A1 of a pharmaceutical composition comprising one or more TKIs, TKIs other than SYK inhibitors, for example, tofacitinib, or a pharmaceutically acceptable salt thereof, let alone of such a pharmaceutical composition comprising one or more TKIs in a local and sustained release biodegradable nanoparticle or microparticle formulation, nor specific formulations and compositions to be administered by intraarticular or intranasal or intraocular route to a subject in need of treatment for medical conditions including inflammatory and allergic diseases such as inflammation of the joints (especially osteoarthritis and crystal-induced arthritis), nasal inflammatory and allergic conditions (especially AERD and EOE).

Similarly US20100168116 discloses SYK tyrosine kinase inhibitors that may be potentially useful in treating diseases resulting from inappropriate activation of mast cells and related inflammatory and allergic responses in the nose. However, there is no disclosure in US US20100168116 of a pharmaceutical composition comprising one or more TKIs, TKIs other than SYK inhibitors, for example, tofacitinib, or a pharmaceutically acceptable salt thereof, let alone of such a pharmaceutical composition comprising one or more TKIs in a local and sustained release biodegradable nanoparticle or microparticle formulation, nor specific formulations and compositions to be administered by intranasal route to a subject in need of treatment for medical conditions. Furthermore, US2010/0168116 describes a dosage restricted to a maximum of 5% w/v which would have a substantially shorter duration of effect compared the composition of the product described herein.

Although WO2012104402A1 discloses that the oral administration of masatinib and formulations disclosed therein may be potentially useful in treating severe persistent asthma, there is no disclosure in WO2012104402A1 of a pharmaceutical composition comprising one or more TKIs in a locally administered and sustained release biodegradable nanoparticle or microparticle formulation, nor specific formulations and compositions to be administered by local administration including inhaled or intranasal route to a subject in need of treatment for other forms of asthma including mild, intermittent, AERD, medication induced, occupational, adult onset, and/or polyposis covered by the methods of use described herein.

While “Guyer B et al. (2004) J Allergy Clin Immunol., 113(2); S28-29. doi:10.1016/j.jaci.2003.12.058” disclose that intranasal dosing of a SYK inhibitors R112 was safe and effectively improved allergic rhinorrhea, in a more recent phase II clinical trial for allergic rhinitis (Clinical Trials.gov Identifier NCT0015089), R112 was however shown as having a lack of efficacy versus placebo. Thus, there is need for a novel application in the art to determine the mechanisms of other TKIs, local, long acting, sustained release formulations in addition to immediate release applications, and consideration for the treatment of mast cell-mediated inflammatory conditions such as OA, crystal-induced arthritis, allergic rhinitis, chronic rhinosinusitis, AERD, EOE, etc.

The methods and compositions to be described herein relate to TKIs, mast cell-mediated inflammatory joint diseases and arthritides, mast cell-mediated inflammatory eye diseases, mast cell-mediated pulmonary diseases, and mast cell-mediated allergic diseases for which the following background information is provided.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1H are representative knee joint sections and graphs illustrating reduction in osteoarthritis pathologies in mice that lack IL12 beta (IL12b), a major inflammatory cytokine involved in several inflammatory diseases including RA. FIG. 1A Mice were induced to develop osteoarthritis by surgically-induced destabilization of the medial meniscus (DMM). FIG. 1B shows the cartilage degradation scores in control or wild-type (WT, open circles) and IL12b-deficient (IL12b−/−, closed circles), assessed using a semi-quantitative scoring system 20-weeks post DMM surgery. FIG. 1C shows the osteophyte score. FIG. 1D shows the synovitis score for the same mice. Statistical analyses were done by unpaired Student's t test. FIG. 1E is representative knee joint sections and graphs illustrating reduction in osteoarthritis pathologies in mice that lack STAT2, a transcription factor downstream of IFN gamma (IFNg) signaling known to induce macrophage activation in several inflammatory diseases including RA. FIG. 1F shows the cartilage degradation scores in control or wild-type (WT, open circles) and STAT2-deficient (Stat2−/−, closed circles), assessed using a semi-quantitative scoring system 20-weeks post DMM surgery. FIG. 1G shows the osteophyte score. FIG. 1H shows the synovitis score for the same mice. Statistical analyses were done by unpaired Student's ttest.

FIG. 2A-2F are representative knee joint sections and graphs illustrating reduction in cartilage damage 20-weeks following DMM surgery in mice lacking specific Fc receptors. FIG. 2A shows representative safranin-o stained knee joint sections from wild-type (WT) and Fc gamma common chain-deficient (Fcerlg−/−) mice. Cartilage damage as evaluated by profound loss of proteoglycans or red staining is indicated by black arrowheads. FIG. 2B shows summed cartilage damage scores for the groups of WT (closed circles) and Fcerlg−/− (closed squares) mice. FIG. 2C shows representative safranin-o stained knee joint sections from wild-type (WT) and activating Fc gamma receptor 3-deficient (Fcgr3−/−) mice. Major cartilage damage as evaluated by profound loss of proteoglycans or red staining is indicated by white block arrowheads and moderate damage is indicated by black arrows. FIG. 2D shows summed cartilage damage scores for the groups of WT (closed circles) and Fcgr3−/− (closed squares) mice. FIG. 2E shows representative safranin-o stained knee joint sections from wild-type (WT) and high affinity IgE receptor Fc epsilon receptor 1 alpha-deficient (Fcerla−/−) mice. Major cartilage damage as evaluated by profound loss of proteoglycans or red staining is indicated by black arrows and moderate damage is indicated by asterisk. FIG. 2F shows summed cartilage damage scores for the groups of WT and Fcerla−/− mice. Statistical analyses were done by unpaired Student's ttest.

FIG. 3A-3D show the results of experiments demonstrating that genetic elimination of MCSF and consequently monocytes/macrophages significantly diminishes osteoarthritis-like pathologies in mice following destabilization of the medial meniscus. FIG. 3A shows representative toluidine blue stained joint-tissue sections from wild-type (Csf^{+/+}) and Csf-deficient (Csf^{-/-}) mice 20-weeks following destabilization of the medial meniscus (DMM) surgery. Arrowheads denote areas of cartilage damage. FIG. 3B-3D are bar graphs showing histological scores of cartilage damage, synovitis and osteophyte formation in mice as described in FIG. 3C, respectively. *P<0.05 and by unpaired Student's ttest.

FIG. 4A-4D show the results of experiments demonstrating that genetic elimination of mast cells reduces murine osteoarthritis severity and reconstitution of mast cells in these mice abrogates the protection conferred by mast cell deficiency. FIG. 4A shows representative knee joint sections stained with safranin-o from control mice (left panel), mast cell deficient (Kit^{W-sh}) mice (middle panel) that received PBS i.e., no mast cells and mast cell reconstituted (right panel) i.e., Kit^{W-sh} mice that received bone marrow-derived mast cells, 20-weeks after DMM surgery. Arrows indicate areas of cartilage damage. FIG. 4B-4D are graphs showing histological scores of cartilage damage, synovitis and osteophyte formation in mice as described in FIG. 4C, respectively. *P<0.05 and **P<0.01 by unpaired Student's ttest.

FIG. 5A-5D shows the results of experiments illustrating the treatment of murine osteoarthritis with the tyrosine kinase inhibitor, imatinib, at doses of 33 mg/kg/day or 100 mg/kg, given orally twice-daily for 12 weeks starting one day after DMM surgery. FIG. 5A shows representative safranin-o stained knee joint sections from vehicle (left panel), imatinib 33 mg/Kg/day (middle panel), and imatinib 100 mg/Kg/day (right panel) treated mice. Arrows indicate areas of cartilage damage. FIG. 5B-5D are graphs showing histological scores of cartilage damage, synovitis and osteophyte formation in vehicle (circles), imatinib 33 mg/Kg/day (squares), and imatinib 100 mg/Kg/day (triangles) treated mice, respectively. Each symbol represents scores from individual mice and line represents the mean values for these scores. *P<0.05, **P<0.01 and ***P<0.001 by unpaired Student's ttest.

FIG. 6A-6B: FIG. 6A shows representative scanning electron microscopy images of PLGA particles without any drug (empty PLGA) or with any of the 3 TKIs tested i.e., imatinib, tofacitinb or dasatinib. The size range for all PLGA formulations were less than 2 um. FIG. 6B is the result of experiments demonstrating the release of drugs from the PLGA encapsulations over time in 5% simulated synovial fluid containing hyaluronic acid as analyzed by mass spectrometry.

FIG. 7A-7G: FIG. 7A-7C is the results of experiments demonstrating treatment of inflammation in early murine osteoarthritis as illustrated by reduction in synovial inflammatory gene expression at 8 weeks following DMM surgery with a sustained release formulation of imatinib (PLGA Imat [PLGA/Imat]), dasatinib (PLGA Dasa [PLGA/Dasa]) or tofacitinib (PLGA Tofa [PLGA/Tofa]). Mice were given intraarticular injections containing 50 ul of these different formulations every 3 weeks for 8 weeks. Control mice received only PLGA particles denoted as PLGA empty in these graphs. FIG. 7A-7B are graphs showing relative mRNA expression of II1b and Adamts4, key pathogenic mediators of osteoarthritis in the synovium of mice described above. Symbols denote individual mice and line represent mean values. FIG. 7C is a graph showing no change in Mmp3 gene expression in the synovium of mice treated with PLGA imat, PLGA Dasa or PLGA Tofa. Control mice received only PLGA particles denoted as PLGA empty in these graphs. *P<0.05 and **P<0.01 by unpaired Student's ttest. FIG. 7D-7G is the results of experiments illustrating reduction in cartilage damage in mice following intraarticular injections of TKIs in a sustained release formulation at 16-weeks following DMM surgery. Mice were given intraarticular injections containing 50 ul of these different formulations every 3 weeks for 16 weeks. Control mice received only PLGA particles denoted as PLGA empty in these graphs. FIG. 7D shows representative knee joint sections stained with safranin-o from mice treated with vehicle (PLGA empty [PLGA/empty]), imatinib (PLGA imat [PLGA/Imat]), dasatinib (PLGA dasa [PLGA/dasa]) or tofacitinib (PLGA tofa [PLGA/tofa]). Asterisk denotes areas of moderate cartilage damage, arrows indicate areas of severe cartilage damage. FIG. 7E-7G are graphs showing histological scores of cartilage damage, synovitis and osteophyte formation in mice described in FIG. 7D, respectively. *P<0.05, and **P<0.01 by unpaired Student's ttest.

FIG. 8A-8B show the results of experiments demonstrating decreased synovial inflammation in mice 10 days after induction of collagen antibody-induced arthritis (CAIA) following treatment with a single intraarticular injection of 50 ul of different TKI/PLGA formulation. FIG. 8A show representative H&E stained knee joint sections from CAIA-challenged mice that received no treatment (PLGA empty), imatinib (PLGA Imat [PLGA/Imat]), dasatinib (PLGA dasa PLGA/dasa]) or tofacitinib (PLGA tofa [PLGA/tofa]). Bottom panels are magnified images denoting synovial inflammation (arrows) in each of these cases. FIG. 8B show the summed synovitis score from knee joint sections of mice described in FIG. 8A. Symbols denote individual mice and bars denote mean values. *P<0.05, **P<0.01 by unpaired Student's ttest.

FIG. 9A-9J show the results of experiments demonstrating that TKI/PLGA formulations effectively reduce local inflammation 24 hrs after initiation of monosodium urate (MSU) crystal-induced model of gouty arthritis. Mice were given a single intraarticular 50 ul injection of individual TKI/PLGA formulation at 4 h after MSU crystal injection in the knees of these mice. FIG. 9A is a Nanostring-based heatmap depicting fold changes of over 300 genes in the local knee joint of mice obtained at 24 hrs after gouty arthritis induction. Fold changes of individual TKI/PLGA treated mice are those over vehicle (PLGA empty [PLGA/empty]) treated mice. I—set of genes whose expression was significantly lower in all three treatment groups compared to vehicle. II—set of genes whose expression was significantly lower in at least one drug treatment group compared to vehicle. III—set of genes whose expression remained unaltered in all three treatment groups relative to vehicle. FIG. 9B-9J shows bar graphs representing examples of genes whose local expression has been lowered following treatment with TKI/PLGA formulation. *P<0.05n and **P<0.01 by unpaired Student's t test.

FIG. 10 is a schematic representation of some of the known receptor tyrosine kinases and cytoplasmic tyrosine kinases inhibited by imatinib, dasatinib, tofacitinib or other tyrosine kinase inhibitors.

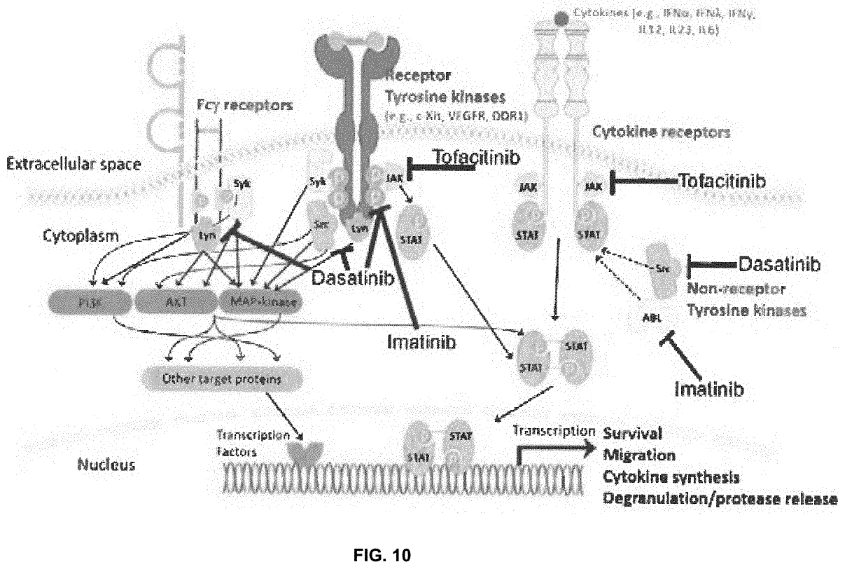

### DETAILED DESCRIPTION OF THE INVENTION

All publications, patents, and patent applications mentioned in this specification are herein incorporated in their entirety for all purposes.

Described herein, the term “mast cell-mediated inflammatory diseases” (also termed “mast cell-associated inflammatory diseases”) broadly refers to inflammatory or allergic diseases or conditions wherein mast cells participate in the induction or propagation or maintenance of inflammation, through selective release of mediators. The term mast cell-mediated inflammatory diseases also refers to conditions wherein mast cells can be activated to rapidly degranulate, not only by IgE and IgE-mediated antigen signaling via the high-affinity receptor for IgE (FcεRI) (which signals via the tyrosine kinase SYK), but also by a diverse group of stimuli that activate receptor tyrosine kinases or signal via non-receptor tyrosine kinases. In addition, the term mast cell-mediated inflammatory diseases refers to conditions where mast cells contribute to the symptomatology of said diseases or critically modulate inflammatory pathways involved in initiation, propagation, tissue remodeling and tissue damage of said diseases. The term mast cell-mediated inflammatory diseases also refers to chronic diseases or conditions that involve aberrant mast cell development or mast cell survival or mast cell activation or mast cell degranulation or mast cell trafficking.

The term “tyrosine kinase inhibitor” (“TKI”) as used herein broadly refers to agents or compounds which are capable of selectively inhibiting tyrosine kinases family of enzymes but do no not target serine or threonine kinases, including those that inhibit MAPK (mitogen-activated protein kinases). The TKI may inhibit tyrosine kinase activity by directly acting on a tyrosine kinase molecule, or it may cooperate with one or more other factors or agents to achieve the desired inhibition. The tyrosine kinase family of enzymes includes both receptor tyrosine kinases and non-receptor tyrosine kinases.

The term “local administration” (or “locally administering”, “local delivery”) as used herein broadly refers to but is not limited to administration to a particular organ, tissue, or body part. Local administration includes but is not limited to intraarticular injection, intralesional injection, intraocular application, intraocular injection, intranasal delivery, sinus delivery, intraauricular delivery, inhaled delivery, swallowed administration, rectal delivery, topical delivery, and other local administration such as those disclosed in this invention is desirable. Local administration of a pharmaceutical composition enables delivery of a level or amount of an agent needed to treat a mast cell-mediated inflammatory disease, or reduce or prevent tissue injury or damage related to mast cell-mediated inflammatory disease, without causing significant negative or adverse side effects to other tissues or organs in the body.

The term “TKI/polymer particles” (also referred to as TKI particles, TKI/polymer, TKI/PLGA, TKI/PLGA particles, polymer/TKI, PLGA/TKI) as used herein broadly refers to a tyrosine kinase inhibitor associated with a biodegradable, bioerodable, biocompatible polymer including but not limited to poly-α-hydroxy acid esters such as, polylactic acid (PLLA or DLPLA), polyglycolic acid, polylactic-co-glycolic acid (PLGA), polylactic acid-co-caprolactone; poly (ester-co-amide) copolymers; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide), polyanhydrides, polyphosphazenes, polyaminoacids etc. TKI/polymer particles can comprise nanoparticles, microparticles, larger particles, and/or combinations of particle sizes. Described herein the terms TKI/PLGA or PLGA/TKI are used interchangeably. For e.g., PLGA particles comprising imatinib can be referred to as Imatinib/PLGA or PLGA/Imatinib or PLGA/Imat or imat/PLGA.

The term “particles” as used herein broadly refers to nanoparticles, microparticles or other sized particles. The particles and TKI/polymer particles described herein can comprise nanoparticles, microparticles, larger particles, or combinations of particle sizes.

The terms “biodegradable” and “biodegradable polymer” refer to biodegradable technology utilized by the bio-medical community. Biodegradable polymers are classified into three groups: medical, ecological, and dual application, while in terms of origin they are divided into two groups: natural and synthetic. The polymer (meaning a material composed of molecules with repeating structural units that form a long chain) is used to encapsulate or form a reservoir for a drug prior to injection in or administration to the body and is frequently based on lactic acid, a compound normally produced in the body, and is thus able to be excreted naturally. The coating is designed for controlled release over a period of time, reducing the number of injections or administrations required and maximizing the therapeutic benefit. Once introduced into the body, biodegradable polymers require no retrieval or further manipulation and are degraded into soluble, non-toxic by-products. Different polymers degrade at different rates within the body and therefore polymer selection can be tailored to achieve desired release rates. The term “biodegradable polymer” also refers to a polymer or polymers which degrade in vivo, and wherein erosion of the polymer or polymers over time occurs concurrent with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an inflammatory condition, tissue injury or damage, or to promote healing of injured or damaged tissue.

The term “therapeutically effective amount” as used herein, refers to the level or amount of agent needed to treat a mast cell-mediated inflammatory disease, or reduce or prevent tissue injury or damage related to mast cell-mediated inflammatory disease without causing significant negative or adverse side effects to the tissue where the pharmaceutical composition is administered.

The term “pharmaceutically acceptable” as used herein means biologically or pharmacologically compatible for in vivo use in animals or humans, and can mean approved by a regulatory agency of the Federal or a state government or listen in the U.S. Pharmacopeia or other generally recognized pharmacopeia for use in animals, and more particularly in humans.

The term “pulmonary conditions” (also referred to as “pulmonary diseases” or “pulmonary conditions”) as used herein broadly refers to but is not limited to asthma including to atopic and nonatopic phenotypes (including but not limited to exercise-induced, nocturnal, occupational, steroid-resistant, cough variant, medication induced, obesity related, adult onset, eosinophilic, perimenopausal), pulmonary fibrosis, cystic fibrosis, pulmonary hypertension, acute respiratory distress syndrome (ARDS), chronic obstructive pulmonary disease (COPD), and aspirin exacerbated respiratory disease (AERD) (Virk, H et al. (2016). Mast cells and their activation in lung disease. Translational Research. Published online. doi:10.1016/j.trsl.2016.01.005).

The term “asthma” as used herein broadly refers to but is not limited to atopic/allergic and nonatopic/allergic phenotypes (including but not limited to exercise-induced, nocturnal, occupational, steroid-resistant, cough variant, medication induced, obesity related, adult onset, eosinophilic, perimenopausal) variants.

The term “joint disease” as used herein broadly refers to but is not limited to diseases affecting joints including but not limited to osteoarthritis, gout, calcium pyrophosphate dihydrate deposition disease, hydroxyapatite crystal deposition disease, rheumatoid arthritis, and other diseases involving a joint or joints.

The term “nasal polyposis disease” as used herein broadly refers to but is not limited to diseases involving the polyps within the nasal passages and sinuses including but not limited to chronic rhinosinusitis or aspirin exacerbated respiratory disease (AERD).

The term “allergic disease” as used herein broadly refers to but is not limited to diseases involving allergic rhinitis, chronic rhinitis, rhinosinusitis, conjunctivitis, ocular allergies, nasal polyps, asthma, aspirin exacerbated respiratory disease (AERD), eosinophilic esophagitis, and other diseases associated with allergic responses. Allergic responses include, but are not limited to, conditions caused by hypersensitivity of the immune system to something in the environment that usually causes little problem in most people.

The systemic administration of tyrosine kinase inhibitors (TKIs), particularly for extended periods of time, can have a number of unwanted side effects including liver toxicities, skin toxicities, cardiotoxicities, bone marrow suppression, or other toxicities. In addition, administration of systemic TKIs for can make patients more susceptible to infections. Accordingly, there is a medical need to extend the local duration of action of TKIs, while reducing the systemic side effects associated with that administration. Thus, there is a need in the art for methods and compositions for the sustained local treatment of pain, discomfort, and symptoms of mast cell inflammation, such as joint pain, ocular pain, sinus pain and congestion, or difficulty breathing, with TKIs that results in clinically tolerable or no measurable systemic toxicities. In addition, there is a medical need to slow, arrest, reverse or otherwise inhibit structural damage to tissues caused by inflammatory diseases such as damage to articular tissues resulting from degenerative arthritides including osteoarthritis (OA), autoimmune arthritides including rheumatoid arthritis (RA), and crystal-induced arthritides including gout, pseudogout, calcific tendonitis, hydroxyapatite crystal arthritis, and other types of crystal-induced arthritis. There is also a medical need to slow, arrest, reverse or otherwise inhibit damage to tissues caused by allergic inflammation such as allergic/inflammatory nasal, ocular, auricular, and pulmonary conditions.

We discovered that mast cells and macrophages activated via tyrosine kinase signaling pathways play critical roles in osteoarthritis pathogenesis (degenerative arthritis) and crystal-induced arthritis. We further discovered that administration of tyrosine kinase inhibitors (TKIs) treats osteoarthritis (OA), gout (a crystal-induced arthritis), and rheumatoid arthritis (RA) using murine models. Further, TKI/PLGA nanoparticle formulations delivered intraarticularly treat osteoarthritis, crystal-induced gouty arthritis and rheumatoid arthritis in murine models. Mast cell-mediated inflammation also plays a pathogenic role in allergic diseases including allergic rhinitis, chronic rhinitis, non-allergic rhinitis, ocular allergies, eosinophilic esophagitis, asthma, AERD, and other pulmonary conditions, EOE, and other inflammatory conditions. The TKI/PLGA particle formulations provided herein are effective at treating inflammation while minimizing the potential side effects of systemic TKI administration, including for example, immunosuppression and infection.

In most embodiments described herein, the compositions specifically utilize tyrosine kinase inhibitors (TKIs) as the active agent, and not inhibitors of non-tyrosine kinases. Other kinase inhibitors include inhibitors that target serine or threonine kinases, including those that inhibit MAPK (mitogen-activated protein kinases). Some receptor tyrosine kinases and signaling tyrosine kinases lead to downstream activation of MAPKs. Although certain TKIs can block signaling pathways that lead to downstream activation of MAPK and/or other serine or threonine kinases, the compositions described herein utilize inhibitors specific to tyrosine kinases as the principal active agent.

Suitable TKIs for use with these methods and compositions may include, but are not limited to, imatinib, afatinib, fostamatinib, axitinib, cediranib, erlotinib, gefitinib, lapatinib, lestaurtinib, neratinib, pazopanib, quizartinib, regorafenib, semaxanib, sorafenib, sunitinib, tivozanib, toceranib, vandetanib, bosutinib, crizotinib, CYT387, dasatinib, nilotinib, ponatinib, ruxolitinib, tofacitinib, baracitinib and vatalanib, as well as the various salts of each these TKIs, derivatives thereof, analogs thereof, and combinations thereof. In some embodiments, the TKI binds to or inhibits a c-KIT receptor or a SYK kinase, or a Src family kinase, or a JAK, or combinations thereof. Preferably, the TKIs are imatinib, dasatinib, tofacitinib, or fostamatinib.

The concentration of the TKI or the TKI content in the formulations of the present invention will depend on the selected route of administration and dosage form, but will generally range from about 10 to about 90% (w/w). The average range of TKI content of the invention is preferably from about 10% to 90% by weight of the pharmaceutical formulation (w/w). In some embodiments, the TKI/polymer particles are about 10%-25% (w/w), about 10%-35% (w/w), about 10%-50% (w/w), about 15%-25% (w/w), about 15%-40% (w/w), about 15%-65% (w/w), about 20%-65% (w/w), about 20%-90% (w/w), about 25%-85% (w/w), about 30%-90% (w/w), about 40%-60% (w/w), about 40%-75% (w/w), about 40%-90% (w/w), about 50%-75% (w/w), about 50%-90% (w/w), about 60%-85% (w/w) and about 60%-90% (w/w). Preferably, the TKI is from about 20% to about 80% by weight of the pharmaceutical formulation, including about 20%, about 25% of about 30%, about 35% of about 40%, about 45%, about 50% about 55%, about 60%, about 65%, about 70% of about 75%, or about 80%. In a particular embodiment, the TKI comprises about 40% by weight of the pharmaceutical formulation (e.g., 30%-50%). In another embodiment, the TKI comprises about 60% by weight of the pharmaceutical formulation. It is understood that these ranges refer to TKI content of all particles in a given population. The TKI content of any given individual particle could be within a standard deviation above or below the mean content of TKI.

The concentration of the TKI or the TKI content in the formulations of the present invention will depend on the selected route of administration and dosage form, but will generally range from 10 to 90% (w/v). The average range of TKI content of the invention is preferably from about 10% to 90% by weight of the pharmaceutical formulation (w/v). In some embodiments, the TKI/polymer particles are about 10%-25% (w/v), about 10%-35% (w/v), about 10%-50% (w/v), about 15%-25% (w/v), about 15%-40% (w/v), about 15%-65% (w/v), about 20%-65% (w/v), about 20%-90% (w/v), about 25%-85% (w/v), about 30%-90% (w/v), about 40%-60% (w/v), about 40%-75% (w/v), about 40%-90% (w/v), about 50%-75% (w/v), about 50%-90% (w/w), about 60%-85% (w/v) and about 60%-90% (w/v). In particular embodiments, the TKI is from about 20% to about 80% by weight of the pharmaceutical formulation, including about 20%, about 25% of about 30%, about 35% of about 40%, about 45%, about 50% about 55%, about 60%, about 65%, about 70% of about 75%, or about 80%. In a particular embodiment, the TKI comprises about 40% (w/v) of the pharmaceutical formulation (e.g., about 30%-50%). In another embodiment, the TKI comprises about 60% (w/v) of the pharmaceutical formulation. It is understood that these ranges refer to TKI content of all particles in a given population. The TKI content of any given individual particle could be within a standard deviation above or below the mean content of TKI.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present invention.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate. Among the useful polysaccharides are, without limitation, calcium alginate, and functionalized celluloses, particularly carboxymethylcellulose esters characterized by being water insoluble, a molecular weight of about 5 kDa to 500 kDa, for example. Other polymers of interest include, without limitation, polyesters, polyethers and combinations thereof which are biocompatible and may be biodegradable and/or bioerodible.

Suitable particles for use with these methods and compositions include PLGA and other polymer-including particles, nanoparticles, microparticles, larger particles, or combinations of particle sizes. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (ester-co-amide) copolymers; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose (or other surfactants), carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkanoates such as polyhydroxy valerate, polyhydroxy butyrate, and the like. In particular embodiments, the biodegradable, bioerodible, biocompatible polymer is PLGA. Other polymers disclosed in U.S. Pat. No. 7,063,748B2 and U.S. Pat. No. 5,702,716 may find use in the present invention. The specifications disclosed in U.S. Pat. No. 7,063,748B2 and U.S. Pat. No. 5,702,716 are herein incorporated in their entirety for all purposes.

The biodegradable polymeric materials which are included to form the matrix are desirably subject to enzymatic or hydrolytic instability. Water-soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups. Equally important to controlling the biodegradation of the polymer and hence the extended release profile of the formulation is the relative average molecular weight of the polymeric composition employed in the pharmaceutical composition. Different molecular weights of the same or different polymeric compositions may be included in the pharmaceutical composition to modulate the release profile. In certain embodiments, the relative average molecular weight of the polymer will range from about 7 to about 120 kDa, usually from about 7 to about 20 kDa, more usually from about 20 to about 60 kDa, more usually from about 50 to about 80 kDa, and more usually from about 70 to about 120 kDa. In a preferred embodiment, the relative average molecular weight of the polymer is about 12 to about 54 kDa. It is understood that these ranges refer to molecular weight of the polymer of all particles in a given population. The molecular weight of the polymer of any given individual particle could be within a standard deviation above or below the molecular weight of the polymer.

TKIs may be contained, dispersed, or embedded within the bulk of the particle matrix, may be contained or loaded within a microsphere particle that encapsulates at least some of the TKIs, and/or may be associated with a nanoparticle or nanosphere. One of skill in the art would appreciate the differences of such particle formulations. For instance, microspheres and nanospheres may have different surface area to volume ratios, which may alter the drug-release characteristics and dosage profiles of a TKI encapsulated therewithin. In other words, a TKI encapsulated by a microsphere may have a release profile that is different from that of a TKI encapsulated by a nanosphere. While a particular amount of nanospheres and microspheres may encapsulate the same amount of a TKI, the nanospheres as compared to the microspheres may have an increased surface area to interact with cells in a host tissue.

In some embodiments, copolymers of glycolic acid (GA) and lactic acid (LA) are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the pharmaceutical composition, where a more flexible composition is desirable for larger geometries. PLGA with a higher content of LA are less hydrophilic, absorb less water and subsequently degrade more slowly, as a consequence of the presence of methyl side groups in PLA making it more hydrophobic than PGA. An exception to this rule is the copolymer 50:50 which exhibits the faster degradation. Broadly the % of poly lactic acid (LA) in the PLGA copolymer is 50-100%, preferably about 15-85%, more preferably about 35-75%. The ratio of lactic acid (LA) to glycolic acid (GA) in the polylactic acid polyglycolic acid (PLGA) copolymer can be 0-100%. In some embodiments, the ratio of LA:GA is about 85:15, the ratio of LA:GA is about 75:25, the ratio of LA:GA is about 65:35, the ratio of LA:GA is about 60:40, the ratio of LA:GA is about 55:45, the ratio of LA:GA is 50:50, the ratio of LA:GA is 45:65, the ratio of LA:GA is 40:60, the ratio of LA:GA is about 35:65, the ratio of LA:GA is about 30:70, the ratio of LA:GA is about 25:75. In a particular embodiment, an approximately 75:25 PLGA copolymer is used. In a particular embodiment, an approximately 50:50 PLGA copolymer is used.

The biodegradable polymer matrix of the present invention may comprise a mixture of two or more biodegradable polymers. For example, the pharmaceutical composition may comprise a mixture of a first biodegradable polymer and a different second biodegradable polymer. One or more of the biodegradable polymers may have terminal acid groups. Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. As discussed herein, the matrix of the pharmaceutical composition may release drug at a rate effective to sustain release of an amount of the TKI for more than one week after administration into desired location such as into the joint, ocular tissue, nasal passage. In certain embodiments, therapeutic amounts of the TKI are released for more than about one month, and even for about six months or more.

Another example of the long acting, biodegradable pharmaceutical composition comprises a TKI with a biodegradable polymer matrix that comprises a single type of polymer. For example, the biodegradable polymer matrix may consist essentially of a polycaprolactone. The polycaprolactone may have a molecular weight between about 10 and about 20 kilodaltons, such as about 15 kilodaltons. These formulations are capable of providing a nearly linear release rate for at least about 70 days, or for at least about 50 days, or for at least about 30 days, or for at least about 15 days. In some embodiments, the TKI/PLGA particles or TKI particles have a mean diameter in the range of about 0.02 to 100 μm, for example, as detected by laser light scattering methods. In some embodiments, the particles have a mean diameter in the range of about 20-100 nm, about 20-200 nm, about 40-400 nm, about 40-600 nm, about 60-800 nm, about 60-1000 nm, about 200 nm-2 μm, about 400 nm-2 μm, about 600 nm-4 μm, about 600 nm-6 μm, about 800 nm-4 μm, about 800 nm-6 μm, about 800 nm-1 μm, about 1 μm-20 μm, about 1 μm-40 μm, about 10 μm-30 μm, about 20 μm-40 μm, about 20 μm-60 μm, about 30 μm-60 μm, about 30 μm-80 μm, about 40 μm-60 μm, about 50 μm-80 μm, about 40 μm-80 μm, about 40 μm-90 μm, about 40 μm-100 μm. It is understood that these ranges refer to the mean diameter of all particles in a given population. The diameter of any given individual particle could be within a standard deviation above or below the mean diameter.

In some embodiments, the TKI/PLGA particles or TKI particles are administered in a formulation having a viscosity in the range of about 2.0 centipoise (cP) to about 4 cP. In some embodiments the formulation has a viscosity in the range of about 2.7 cP to about 3.5 cP. In some embodiments, the TKI/PLGA particles or TKI particles are administered in a formulation having a viscosity in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TKI/PLGA particle or TKI particle formulations are administered at a TKI dose in the range of about 10 mg to about 2500 mg and in a formulation having a viscosity in the range of about 2.7 cP to about 3.5 cP. In some embodiments, TKI/PLGA particle or TKI particle formulations are administered at a TKI dose in the range of about 10 mg to about 2500 mg and in a formulation having a viscosity of in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TKI/PLGA particle or TKI particle formulations are administered at a TKI dose in the range of about 10 mg to about 500 mg and in a formulation having a viscosity of about 3.0 cP. In some embodiments, the TKI PLGA particles or TKI particles are administered as a suspension having a viscosity in the range of about 2.0 centipoise (cP) to about 4 cP. In some embodiments the formulation has a viscosity in the range of about 2.7 cP to about 3.5 cP. In some embodiments, the TKI/PLGA particles or TKI particles are administered as a suspension having a viscosity in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TKI/PLGA particle or TKI particle formulations are administered at a TKI dose in the range of about 10 mg to about 2500 mg and as a suspension having a viscosity in the range of about 2.7 cP to about 3.5 cP. In some embodiments, TKI/PLGA particle or TKI particle formulations are administered at a TKI dose in the range of about 10 mg to about 50 mg and as a suspension having a viscosity of in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TKI/PLGA particle or TKI particle formulations are administered at a TKI dose in the range of about 10 mg to about 2500 mg and as a suspension having a viscosity of about 3.0 cP.

In some embodiments, the TKI/PLGA particle or TKI particle formulations are administered at a TKI dose in the range of about 10 to about 20 mg, or about 10 to about 50 mg, or about 25 to about 50 mg, or about 50 to about 100 mg, or about 75 to about 150 mg, or about 100 to about 250 mg, or about 200 to about 400 mg, or about 250 to about 500 mg, or about 300 to about 600 mg, or about 500 to about 1000 mg, or about 750 to about 1500 mg, or about 1000 to about 2000 mg, or about 1500 to about 2500 mg.

Various methods may be used to associate TKIs in polymers to form particles, including, but not limited to, forming the nanoparticles or microparticles in the presence of a solution comprising the TKI. Examples of these methods are described below.

The manufacture of PLGA particles or methods of making biodegradable polymer nanoparticles are known in the art. PLGA particles are commercially available from a number of sources and/or can be made by, but not limited to, spray drying, solvent evaporation, phase separation, fluidized bed coating or combinations thereof. If not purchased from a supplier, then the biodegradable PLGA copolymers may be prepared by the procedure set forth in U.S. Pat. No. 4,293,539, the disclosure of which is hereby incorporated by reference in its entirety for all purposes. Ludwig prepares such copolymers by condensation of lactic acid and glycolic acid in the presence of a readily removable polymerization catalyst (e.g., a strong acid ion-exchange resin such as Dowex HCR-W2-H). However, any suitable method known in the art of making the polymer can be used.

In the coacervation process, a suitable biodegradable polymer is dissolved in an organic solvent. Suitable organic solvents for the polymeric materials include, but are not limited to acetone, halogenated hydrocarbons such as chloroform and methylene chloride, aromatic hydrocarbons such as toluene, halogenated aromatic hydrocarbons such as chlorobenzene, and cyclic ethers such as dioxane. The organic solvent containing a suitable biodegradable polymer is then mixed with a non-solvent such as silicone based solvent. By mixing the miscible non-solvent in the organic solvent, the polymer precipitates out of solution in the form of liquid droplets. The liquid droplets are then mixed with another non-solvent, such as heptane or petroleum ether, to form the hardened nanoparticles. The nanoparticles are then collected and dried. Process parameters such as solvent and non-solvent selections, polymer/solvent ratio, temperatures, stirring speed and drying cycles are adjusted to achieve the desired particle size, surface smoothness, and narrow particle size distribution.

In the phase separation or phase inversion procedures entrap dispersed agents in the polymer to prepare nanoparticles. Phase separation is similar to coacervation of a biodegradable polymer. By addition of a non-solvent such as petroleum ether to the organic solvent containing a suitable biodegradable polymer, the polymer precipitates from the organic solvent to form nanoparticles.

In the salting out process, a suitable biodegradable polymer is dissolved in an aqueous miscible organic solvent. Suitable water miscible organic solvents for the polymeric materials include, but are not limited to acetone, acetonitrile, and tetrahydrofuran. The water miscible organic solvent containing a suitable biodegradable polymer is then mixed with an aqueous solution containing salt. Suitable salts include, but are not limited to electrolytes such as magnesium chloride, calcium chloride, or magnesium acetate and non-electrolytes such as sucrose. The polymer precipitates from the organic solvent to form nanoparticles, which are collected and dried. Process parameters such as solvent and salt selection, polymer/solvent ratio, temperatures, stirring speed and drying cycles are adjusted to achieve the desired particle size, surface smoothness, and narrow particle size distribution.

Alternatively, the nanoparticles or microparticles or other particles may be prepared by the process of Ramstack et al, 1995, described in published international patent application WO1995013799A1, the disclosure of which is incorporated herein in its entirety. The Ramstack et al. process essentially provides for a first phase, including an active agent and a polymer, and a second phase, that are pumped through a static mixer into a quench liquid to form nanoparticles containing the active agent. The first and second phases can optionally be substantially immiscible and the second phase is preferably free from solvents for the polymer and the active agent and includes an aqueous solution of an emulsifier. In the spray drying process, a suitable biodegradable polymer is dissolved in a suitable solvent and then sprayed through nozzles into a drying environment provided with sufficient elevated temperature and/or flowing air to effectively extract the solvent.

Alternatively, a suitable biodegradable polymer can be dissolved or dispersed in supercritical fluid, such as carbon dioxide. The polymer is either dissolved in a suitable organic solvent, such as methylene chloride, prior to mixing in a suitable supercritical fluid or directly mixed in the supercritical fluid and then sprayed through a nozzle. Process parameters such as spray rate, nozzle diameter, polymer/solvent ratio, and temperatures, are adjusted to achieve the desired particle size, surface smoothness, and narrow particle size distribution.

In a fluidized bed coating, the drug is dissolved in an organic solvent along with the polymer. The solution is then processed, e.g., through a Wurster air suspension coating apparatus to form the final microcapsule product.

The nanoparticles can be prepared in a size distribution range suitable for local infiltration or injection. The diameter and shape of the nanoparticles can be manipulated to modify the release characteristics. In addition, other particle shapes, such as, for example, cylindrical shapes, can also modify release rates of a sustained release TKI/PLGA nanoparticle or TKI particle by virtue of the increased ratio of surface area to mass inherent to such alternative geometrical shapes, relative to a spherical shape. The nanoparticles have a volumetric mean diameter ranging between about 0.5 to 500 microns. In a preferred embodiment, the nanoparticles have a volumetric mean diameter of between 10 to about 100 microns.

Biodegradable polymer nanoparticles that deliver sustained release TKI may be suspended in suitable aqueous or non-aqueous carriers which may include, but is not limited to water, saline, pharmaceutically acceptable oils, low melting waxes, fats, lipids, liposomes and any other pharmaceutically acceptable substance that is lipophilic, substantially insoluble in water, and is biodegradable and/or eliminatable by natural processes of a patient's body. Oils of plants such as vegetables and seeds are included. Examples include oils made from corn, sesame, cannoli, soybean, castor, peanut, olive, arachis, maize, almond, flax, safflower, sunflower, rape, coconut, palm, babassu, and cottonseed oil; waxes such as carnauba wax, beeswax, and tallow; fats such as triglycerides, lipids such as fatty acids and esters, and liposomes such as red cell ghosts and phospholipid layers.

As the biodegradable PLGA polymers, and other biodegradable polymers, undergo gradual bio-erosion at the target site for example within the joint, the TKI is released to the inflammatory site. The pharmacokinetic release profile of TKI by the biodegradable PLGA polymer may be first order, zero order, bi- or multiphasic, to provide desired treatment of inflammatory related pain. In any pharmacokinetic event, the bio-erosion of the polymer and subsequent release of TKI may result in a controlled release of TKI from the polymer matrix.

### Excipients

The release rate of TKI from a PLGA biodegradable polymer matrix or other polymer matrices can be modulated or stabilized by adding a pharmaceutically acceptable excipient to the formulation. An excipient may include any useful ingredient added to the biodegradable polymer depot that is not a corticosteroid or a biodegradable polymer. Pharmaceutically acceptable excipients may include without limitation lactose, dextrose, sucrose, sorbitol, mannitol, starches, gum acacia, calcium phosphate, alginates, tragacanth, gelatin, calcium silicate, microcrystalline cellulose, PEG, polysorbate 20, polysorbate 80, polyvinylpyrrolidone, cellulose, water, saline, syrup, methyl cellulose, and carboxymethyl cellulose. An excipient for modulating the release rate of TKI from the biodegradable PLGA drug depot may also include without limitation pore formers, pH modifiers, solubility enhancers, reducing agents, antioxidants, and free radical scavengers.

Size of PLGA particles and TKI particles: Nanoparticles and microspheres. PLGA particles and TKI particles can either be made micro-scale or nano-scale. Nanoparticles are particles with sizes smaller than 1 μm. Their extremely small size results in a high ratio of surface area to volume. This ratio promotes a high degree of surface adsorption by drugs, proteins, and other molecules. It also allows for increased interaction with other particles, which leads to changes in physical properties. The small size of nanoparticles provides other benefits, as well. For example, nanoparticles can be made aggregation free, making them useful for intravenous or systemic drug delivery. They can additionally enter all cells via pinocytosis such that not just professional phagocytes can take up the particle. Furthermore, nanoparticles can be manufactured and produced in sterile form.

Microspheres (sizes can vary from 1 μm-1 mm) are also frequently used in drug delivery systems and have their own advantages. Because microspheres are larger than nanoparticles, they are able to encapsulate a larger amount of drugs or other molecules. However, polymeric microspheres themselves are known to cause acute inflammation, followed by an indolent chronic inflammatory response in 7-14 days. Therefore, administering very high doses of such drug-loaded PLGA microspheres could have serious adverse effects.

### Administration of TKI/PLGA Particles and TKI Particles

In one embodiment the TKI/PLGA particle or TKI particle formulations are suitable for administration, for example, local administration by injection into a site at or near the site of a patient's pain and/or inflammation. The TKI/PLGA particle or TKI particle formulations provided herein are effective in slowing, arresting, reversing or otherwise inhibiting structural damage to tissues associated with progressive disease with minimal long-term side effects of TKI/PLGA particle or TKI particle administration, including for example, prolonged suppression of the immune system. The TKI/PLGA particle or TKI particle formulations provided herein are also effective at reducing a patient's joint pain.

In another embodiment, a sustained release form of TKI/PLGA particles or TKI particles is administered locally to treat inflammation and attenuate structural damage. Local administration of a TKI/PLGA particle formulation can occur, for example, by injection into the intraarticular space or peri-articular space at or near the site of a patient's pain. Local administration can also include but is not limited to intraocular, intranasal, intra-auricular, inhaled, swallowed, intra-rectal, topical, or other local route of administration as disclosed in this invention. When intra-articularly delivered TKI is incorporated into a PLGA biodegradable polymer for sustained release into a joint at a dosage that does not induce TKI-associated systemic toxicity, preferred loadings of the TKI are about 10-60% (w/w) of the PLGA particle.

In certain other embodiments, the formulation additionally contains an immediate release component. The immediate release component can be provided in various forms, for example as non-encapsulated TKI (e.g., not incorporated within a polymeric matrix), a bimodal particle size distribution in which the immediate release particles have a much smaller particle size/higher effective surface area to provide for more rapid release, or can be in the form of particles in which the biodegradable polymer is designed to degrade more rapidly, In certain particular embodiments of the invention, a sustained release form of TKI/PLGA particles is administered (e.g., by single injection or as sequential injections) into an intra-articular space for the treatment of inflammation, for example, due to osteoarthritis, rheumatoid arthritis, gouty arthritis, pseudogout arthritis, hydroxyapatite crystal arthritis, other crystal arthritis, and/or other joint disorders, or into local tissues affected by bursitis, tenosynovitis, epicondylitis, synovitis and/or other disorders. When intra-articularly delivered TKI is incorporated into a PLGA biodegradable polymer for sustained release into a joint at a dosage that does not induce TKI-associated systemic toxicity, preferred loadings of the TKI are about 10-90% (w/w) of the TKI/PLGA nanoparticle or microparticle or other particles.

In certain preferred embodiments of the invention, a sustained release form of TKI/PLGA particles or TKI particles is administered (e.g., by single injection or as sequential injections) into an intra-articular space to slow, arrest, reverse or otherwise inhibit structural damage to tissues associated with progressive disease such as, for example, the damage to cartilage associated with progression of osteoarthritis. In other preferred embodiments, local administration can include but is not limited to intraocular, intranasal, intra-auricular inhaled, and swallowed delivery of the TKI/PLGA or TKI particles. The TKI/PLGA particles described herein are also useful in the treatment of a systemic disorder for which TKI treatment would be required or otherwise therapeutically beneficial.

In some embodiments, the population of TKI/PLGA particles or TKI particles, the controlled or sustained release TKI/PLGA particle or TKI particle preparation or formulation is administered as one or more intra-articular injections. In some embodiments, the population of TKI/PLGA particles, the controlled or sustained release TKI/PLGA particle or TKI particle preparation or formulation is administered as one or more local injections at the site of pain. In some embodiments, the patient has osteoarthritis, rheumatoid arthritis, psoriatic arthritis, reactive arthritis, acute gouty arthritis, acute pseudogout arthritis, and/or other arthritis or synovitis. In some embodiments, the patient has acute bursitis, sub-acute bursitis, acute nonspecific tenosynovitis, calcific tendonopathy, Milwaukee shoulder, and/or epicondylitis. The invention also provides methods of slowing, arresting or reversing progressive structural tissue damage associated with chronic inflammatory disease in a patient by administering to said patient a therapeutically effective amount of a population of TKI/PLGA particles described herein. It is contemplated that whenever appropriate, any embodiment of the present invention can be combined with one or more other embodiments of the present invention, even though the embodiments are described under different aspects of the present invention.

Recently, the clinical problem of “crystal-induced pain” caused by the substance remaining in the joint has begun to receive considerable attention (Horisawa, E et al. (2002). Size-dependency of DL-lactide/glycolide copolymer particulates for intra-articular delivery system on phagocytosis in rat synovium. Pharmaceutical research, 19(2), 132-139 DOI:10.1023/A:1014260513728). The mechanism by which crystal-induced pain is generated in the human joint cavity remains unknown. Since the pain has not arisen so often with aqueous drug preparations, it is thought that the bioincompatibility and the physicochemical properties (i.e., diameter, shape) of the drug particles are closely related to the pain induction (Horisawa, E et al. (2002). Size-dependency of DL-lactide/glycolide copolymer particulates for intra-articular delivery system on phagocytosis in rat synovium. Pharmaceutical research, 19(2), 132-139 DOI:10.1023/A:1014260513728). Additionally, it was found that steroidal microspheres prepared with several polymeric materials were phagocytosed by the synovial activated cells depending on their particle size. They assessed that the irritancy with synovial tissues depended on the biocompatibility of the colloidal particles (Ratcliffe, J H et al. (1984). Preparation and evaluation of biodegradable polymeric systems for the intra-articular delivery of drugs. Journal of pharmacy and pharmacology, 36(7), 431-436. DOI: 10.1111/j.2042-7158.1984.tb04419.x).

PLGA nanoparticles containing TKI or other drug molecules might be more suitable for drug delivery to inflamed synovium rather than the larger microparticles/microspheres taking into consideration, prolonged pharmacologic efficacy, ability to penetrate synovium and that they are less likely to be inflammatory in and of themselves.

In yet a further embodiment, the TKI/PLGA particle or TKI particle formulations are suitable for local or topical administration, (i.e., local, organ-specific delivery) by means of conventional topical formulations, such as liquids, solutions, suspensions, gels, sprays, mists, drops, for the nose, eyes, ears, inhalation for pulmonary efficacy, and swallowed for local oropharyngeal and esophageal efficacy. The TKI/PLGA particle and TKI particle formulations provided herein are effective in slowing, arresting, reversing or otherwise inhibiting damage to tissues associated with progressive disease with minimal side effects of TKI/PLGA particle and TKI particle administration, including for example, prolonged suppression of the immune system. The TKI/PLGA particle and TKI particle formulations provided herein are also effective at reducing the patient's discomfort, for example allergic reactions, nasal congestion, itchy/water eyes, pain in the nose or eyes or ears, shortness of breath, gastroesophageal reflux (GERD) or dysphagia as seen in ophthalmic allergic/inflammatory disorders (including conjunctivitis and uveitis), otic allergic/inflammatory disorders, nasal allergic/inflammatory disorders, nasal polyps, rhinitis, sinusitis, rhinosinusitis, reversible airway obstruction, adult respiratory distress syndrome, asthma, chronic obstructive pulmonary disease, aspirin exacerbated respiratory disease and bronchitis.

In another embodiment, a sustained release form of TKI/PLGA particles or TKI particles is administered locally to treat inflammation and attenuate structural damage. Local administration of a TKI/PLGA particle formulation can occur, for example, by nasal spray or gel, eye/ear/nose drops or gel, inhaled, swallowed, or injected, depending on the patient's symptoms and affected organs. When intranasal or intraocular delivered TKI is incorporated into a PLGA biodegradable polymer for sustained release into the nose at a dosage that does not induce TKI-associated systemic toxicity, preferred loadings of the TKI are about 10%-90% (w/w) of the PLGA particle. In a preferred embodiment, the TKI comprises about 40% by weight of the pharmaceutical formulation (e.g., from about 30% to about 50%). In some embodiments, the TKI comprises from about 10% to about 20% of the pharmaceutical formulation, or about 20% to about 30% of the pharmaceutical formulation, or about 30% to about 40% of the pharmaceutical formation, or about 40% to about 50% of the pharmaceutical formation, or about 50% to about 60% of the pharmaceutical formulation, or about 60% to about 70% of the pharmaceutical formulation, or about 70% to about 80% of the pharmaceutical formulation, or about 80% to about 90% of the pharmaceutical formulation.

In certain other embodiments, the formulation additionally contains an immediate release component. The immediate release component can be provided in various forms, for example as non-encapsulated TKI (e.g., not incorporated within a polymeric matrix), a bimodal particle size distribution in which the immediate release particles have a much smaller particle size/higher effective surface area to provide for more rapid release, or can be in the form of particles in which the biodegradable polymer is designed to degrade more rapidly, In certain particular embodiments of the invention, a sustained release form of TKI/PLGA particles is administered (e.g., by nasal spray or gel, eye/ear drops or gel, inhaled, swallowed, or injected) into the nose, eyes, ears, lungs, or esophagus, respectively, for the treatment of inflammation, for example, due to allergic/non-allergic/chronic rhinitis, rhinosinusitis, conjunctivitis, uveitis, EoE, asthma, other pulmonary conditions, or joint disease. When intra-articularly delivered TKI is incorporated into a PLGA biodegradable polymer for sustained release into a joint at a dosage that does not induce TKI-associated systemic toxicity, preferred loadings of the TKI are about 10%-90% (w/w) of the nanoparticle. In a preferred embodiment, the TKI comprises about 40% by weight of the pharmaceutical formulation (e.g., 30%-50%). In some embodiments, the TKI comprises from about 10% to about 20% of the pharmaceutical formulation, or about 20% to about 30% of the pharmaceutical formulation, or about 30% to about 40% of the pharmaceutical formation, or about 40% to about 50% of the pharmaceutical formation, or about 50% to about 60% of the pharmaceutical formulation, or about 60% to about 70% of the pharmaceutical formulation, or about 70% to about 80% of the pharmaceutical formulation, or about 80% to about 90% of the pharmaceutical formulation.

In certain particular embodiments of the invention, a sustained release form of TKI/PLGA particles or TKI particles is administered (e.g., by single injection or as sequential injections or by single administration or by sequential administration) into the nose, eyes, ear, lungs, esophagus, gastrointestinal tract, or joint, to slow, arrest, reverse or otherwise inhibit damage to the nasal mucosal tissues associated with progressive inflammation associated with allergic/non allergic/chronic rhinitis, conjunctivitis, other eye disease, asthma, other lung disease, or joint disease. The TKI/PLGA particles described herein are also useful in the treatment of a systemic disorder for which TKI treatment would be required or otherwise therapeutically beneficial.

In some embodiments, the population of TKI/PLGA particles or TKI particles, the controlled or sustained release TKI/PLGA or TKI particle preparation or formulation is administered as one or more topical, local, organ specific administrations into the nose, eyes, ears, lungs, or esophagus. In some embodiments, the population of TKI/PLGA particles, the controlled or sustained release TKI/PLGA particle or TKI particle preparation or formulation is administered as one or more local ways of administration at the site of discomfort. In some embodiments, the patient has uveitis or allergic conjunctivitis, allergic/non allergic rhinitis, chronic rhinitis, chronic rhinosinusitis, nasal polyps, and asthma, or other pulmonary condition. The invention also provides methods of slowing, arresting or reversing progressive structural tissue damage associated with chronic inflammatory disease in a patient by administering to said patient a therapeutically effective amount of a population of TKI/PLGA particles described herein. It is contemplated that whenever appropriate, any embodiment of the present invention can be combined with one or more other embodiments of the present invention, even though the embodiments are described under different aspects of the present invention.

In other embodiments dosage forms for nasal or inhaled administration may conveniently be formulated as aerosols, solutions, drops, gels or dry powders.

In a particular embodiment, dosage forms for topical administration to the nasal cavity (nasal administration) include pressurized aerosol formulations, powder and aqueous formulations administered to the nose by pressurized pumps. Formulations which are non-pressurized and adapted for nasal administration are also of interest. Suitable formulations contain water as the diluent or carrier for this purpose. Aqueous formulations for administration to the nose may be provided with conventional excipients such as buffering agents, tonicity modifying agents and the like. Aqueous formulations may also be administered to the nose by nebulisation. Other particular delivery systems include drops, unit dose containers, squeeze bottles, metered-dose pumps sprays, airless and preservative free sprays, compressed air nebulizers, powder dosage forms, insuflators, multi-dose powder systems, pressurized MDIs, nasal gel and all those described in the review by Kublik (Kublik H and Vifgren M T. (1998) Nasal delivery systems and their effect on deposition and absorption. Advanced Drug Delivery Review 29; 157-177 PMID:10837586).

Dosage forms for nasal administration are provided in a metered dose device. The dosage form may be provided as a fluid formulation for delivery from a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. In one embodiment, the fluid dispenser is of the general type described and illustrated in WO2005044354A1. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. Another preferred fluid dispenser is of the general type illustrated in FIGS. 30-40 of WO2005044354A1. Additional preferred dispensers include all devices discussed in the review by Djupesland (Djupesland P G. (2013) Nasal drug delivery devices: characteristics and performance in a clinical perspective a review. Drug Deliv and Transl. Res. 3:42-62. DOI 10.1007/s13346-012-0108-9). Other preferred dispensers similar to and including but not limited to FLONASE nasal spray available from GlaxoSmithKline of the United Kingdom; NASONEX nasal spray available from Schering Corporation of Kenilworth, N.J.; and ASTELIN nasal spray available from MedPointe Pharmaceuticals of Somerset, N.J. All of these products deliver topical formulations via conventional pump-sprayers available from suppliers such as Pfeiffer of Germany; Saint-Gobain Calmar of France, or Valois of France, nasal spray pumps having unit dose systems for nasal powder formulations available from Aptar Inc., (Crystal Lake, Ill.); breath-powered nasal delivery technology available from OptiNose Inc., (Yardley, Pa.); TriVair™ “nasal straw” delivery technology available from Trimel Inc., (Mississauga, Ontario); MicroDose™ Dry Powder Inhaler (DPI), MicroDose™ Dry Powder Nebulizer (DPN), and “electric” atomizing nasal applicators available from MicroDoseTherapeutx Inc., (Monmouth Junction, N.J.); and monodose insufflators available from MIAT S.p.A. (Milan, Italy). Additionally intranasal methods of application and delivery included in US20140227326A1, U.S. Pat. No. 6,297,227B1, WO1999049923A1, and CN101015559 are hereby incorporated by reference in their entirety for all purposes.

In a particular embodiment the particles for intranasal delivery have a size bigger than 9-10 microns because they can be trapped in the nasal cavity, whereas too fine particles having size below 5 microns are usually inhaled directly into the lungs and would be optimal for inhaled formulations. Other embodiments and size specifications are determined by route of delivery as described in: Majgainya, S et al. (2015) Novel approach for nose-to-brain drug delivery bypassing blood brain barrier by pressurized olfactory delivery device. J App Pharm 7; 148-163. ISSN 19204159; Surber, C et al. (2011) Nasal drug delivery in humans. Curr. Probl. Dermatol. 40; pp 20-35 doi: 10.1159/000321044; and Kumar, A et al., (2016). Nasal nanotechnology: revolution for efficient therapeutics delivery. Drug Delivery. 23: pp 681-693 doi: 10.3109/10717544.2014.920431.

In another embodiment, dosage forms for inhaled administration, for the use of asthma or other pulmonary disease or conditions, may conveniently be formulated as aerosols or dry powders. For compositions suitable and/or adapted for inhaled administration, it is preferred that the compound or salt of formula I is in a particle-size-reduced form, and more preferably the size-reduced form is obtained or obtainable by micronisation. The preferable particle size of the size-reduced (e. g., micronised) compound or salt or solvate is defined by a D50 value of about 0.5 to about 10 microns (for example as measured using laser diffraction). Aerosol formulations, e.g., for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted. Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,I,2,3,3,3-heptafluoropropane and I,I,I,2-tetrafluoroethane. The aerosol dosage forms can also take the form of a pump-atomiser. The pressurized aerosol may contain a solution or a suspension of the active compound. This may require the incorporation of additional excipients e. g., co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or the particle mass characteristics (amount and/or profile) of the formulation. For pharmaceutical compositions suitable and/or adapted for inhaled administration, one embodiment is a dry powder inhalable composition. Such a composition can comprise a powder base such as lactose, glucose, trehalose, mannitol or starch, the compound of formula I or salt or solvate thereof (preferably in particle-size-reduced form, e.g., in micronised form), and optionally a performance modifier such as L-leucine or another amino acid, and/or metals salts of stearic acid such as magnesium or calcium stearate. Preferably, the dry powder inhalable composition comprises a dry powder blend of lactose and the compound of TKI/polymer particle or salt thereof. The lactose is preferably lactose hydrate e.g., lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g., 10-1000 microns e.g., 30-1000 microns) in diameter, and/or 50% or more of the lactose particles being less than 500 microns (e. g., 10-500 microns) in diameter. More preferably, the particle size of the lactose is defined by 90% or more of the lactose particles being less than 300 microns (e.g., 10-300 microns e.g., 50-300 microns) in diameter, and/or 50% or more of the lactose particles being less than 100 microns in diameter. Optionally, the particle size of the lactose is defined by 90% or more of the lactose particles being less than 100 200 microns in diameter, and/or 50% or more of the lactose particles being less than 40-70 microns in diameter. It is preferable that about 3 to about 30% (e.g., about 10%) (by weight or by volume) of the particles are less than 50 microns or less than 20 microns in diameter. For example, without limitation, a suitable inhalation-grade lactose is E9334 lactose (10% nes).

Optionally, in particular for dry powder inhalable compositions, a pharmaceutical composition for inhaled administration can be incorporated into a plurality of sealed dose containers (e.g., containing the dry powder composition) mounted longitudinally in a strip or ribbon inside a suitable inhalation device. The container is rupturable or peel-openable on demand and the dose of e.g., the dry powder composition can be administered by inhalation via the device such as the DISKUS® device (GlaxoSmithKline). Other dry powder inhalers are well known to those of ordinary skill in the art, and many such devices are commercially available, with representative devices including Aerolizer® (Novartis), Airrnax™ (IVAX), ClickHaler® (Innovata Biomed), Diskhaler® (GlaxoSmithKline), Accuhaler (GlaxoSmithKline), Easyhaler® (Orion Pharma), Eclipse™ (Aventis), Flow Caps® (Hovione), Handihaler® (Boehringer Ingelheim), Pulvinal® (Chiesi), Rotahaler® (GlaxoSmithKline), Skye Haler™ or Certihaler™ (SkyePharma), Twisthaler® (Schering Corp.), Turbuhaler® (AstraZeneca), Ultrahaler® (Aven tis), and the like.

In some embodiments, a microparticle/nanoparticle coating may be applied using a spray method. Spray may involve spraying or applying an atomized or aerosolized form of the composition topically to form a sheet, film, or coating.

The coatings created by spraying may have a thickness of about 20 microns. Alternatively, the coatings may have a thickness of at least about 2.5 microns, at least about 5 microns, at least about 10 microns, at least about 15 microns, at least about 25 microns, at least about 30 microns, at least about 35 microns, at least about 50 microns, at least about 75 microns, at least about 100 microns, at least about 125 microns, at least about 150 microns, at least about 175 microns, at least about 200 microns, at least about 225 microns, at least about 250 microns, at least about 300 microns, at least about 350 microns, at least about 400 microns, at least about 450 microns, or at least about 500 microns. Any other thickness consistent with the coating's function may also be used. The porosity and stability of the sprayed coating may vary as a function of the concentration of alcohol(s) (e.g., ethanol) in the composition. For instance, the porosity may increase, and the stability may decrease (e.g., increased fragility and brittleness of the coating or sheet) as the alcohol concentration increases. Stability may be measured as a function of sheet or coating integrity.

In another embodiment, solutions intended for topical administration to the eye, the concentration of the TKI is preferably about 10 to 90% (w/v or w/w). In a preferred embodiment, the TKI comprises about 40% by weight of the pharmaceutical formulation (e.g., about 30%-50%). The topical compositions of the present invention are prepared according to conventional techniques and contain conventional excipients in addition to one or more TKI compounds of formula. A general method of preparing eye drop compositions is described below: One or more TKI compounds of formula (I) and a tonicity-adjusting agent are added to sterilized purified water and if desired or required, one or more excipients. The tonicity-adjusting agent is present in an amount sufficient to cause the final composition to have an ophthalmically acceptable osmolality (generally about 150-450 mOsm, or about 100-500 mOsm, or preferably 250-350 mOsm). Conventional excipients include preservatives, buffering agents, chelating agents or stabilizers, viscosity-enhancing agents and others. The chosen ingredients are mixed until homogeneous. After the solution is mixed, pH is adjusted (typically with NaOH or HCl) to be within a range suitable for topical ophthalmic use, preferably within the range of 4.5 to 8. Many ophthalmically acceptable excipients are known, including, for example, sodium chloride, mannitol, glycerin or the like as a tonicity-adjusting agent; benzalkonium chloride, polyquaternium-I or the like as a preservative; sodium hydrogen phosphate, sodium dihydrogen phosphate, boric acid or the like as a buffering agent; edetate disodium or the like as a chelating agent or stabilizer; polyvinyl alcohol, polyvinyl pyrrolidone, polyacrylic acid, to polysaccharide or the like as a viscosity-enhancing agent; and sodium hydroxide, hydrochloric acid or the like as a pH controller.

In one aspect, for example intraarticular injection, a TKI/PLGA particle or TKI particle formulation provides at least two weeks, preferably at least three weeks, including up to and beyond about 2 days, or about 3 days, or about 5 days, or about 7 days, or about 15 days, or about 30 days, or about 60 days, or about 90 days, or about 120 days of a sustained, steady state release of TKI. In one aspect, a TKI/PLGA particle or TKI particle formulation is provided wherein the TKI/PLGA particle or TKI particle formulations provide at least two weeks, preferably at least three weeks, including up to and beyond about 2 days, about 3 days, about 5 days, about 7 days, or about 15 days, or about 30 days, or about 60 days, or about 90 days, or about 120 days of a sustained, steady state release of TKI at a rate that does not have adverse effects. The duration of the release of TKI from the TKI/PLGA particles or TKI particles can vary in relation to the total number of TKI/PLGA particles contained in a given formulation.

In some embodiments, the TKI/PLGA particle or TKI particle formulation retains sustained efficacy even after the TKI is no longer resident at the site of administration, for example, in the intra-articular space, and/or after the TKI is no longer detected in the systemic circulation. The TKI/PLGA particle or TKI particle formulation retains sustained efficacy even after the TKI/PLGA or TKI microparticle formulation is no longer resident at the site of administration, for example, in the intra-articular space, and/or the released TKI is no longer detected in the systemic circulation. The TKI/PLGA particle formulation retains sustained efficacy even after the TKI/PLGA particle formulation ceases to release therapeutically effective amounts of TKI. For example, in some embodiments, the TKI released by the TKI/PLGA microparticle formulation retains efficacy for at least one week, at least two weeks, at least three weeks, at least four weeks, at least five weeks, at least six weeks, at least seven weeks, at least eight weeks, at least nine weeks, at least twelve weeks, or more than twelve-weeks post-administration. In some embodiments, the TKI released by the TKI/PLGA particle formulation retains efficacy for a time period that is at least 1.1 times as long, at least 1.2 times as long, at least 1.3 times as long, at least 1.4 times as long, at least 1.5 times as long, at least 1.6 times as long, at least 1.7 times as long, at least 1.8 times as long, at least 1.9 times as long, twice as long, at least 2.5 times as long, at least three times as long, at least four times as long, at least five times as long, at least ten times as long, at least fifteen times as long, at least twenty times as long, at least thirty times as long, at least forty times as long, at least fifty times as long, at least seventy-five times as long, at least one hundred times as long, at least two-hundred, at least three hundred, at least five-hundred, at least one-thousand, at least 2500, at least 10000, at least 50000 or more than 50000 times as long as the residency period for the TKI and/or the TKI/PLGA particle formulation.

In some embodiments, a controlled- or sustained-release TKI/PLGA formulation is provided wherein formulation may or may not exhibit an initial rapid release in addition to the sustained, steady state release of TKI for a second length of time of at least 2 days, at least 3 days, at least 4 days, at least 5 days, at least one week, at least two weeks, preferably at least three weeks, including up to and beyond 3 days, 5 days, 7 days, 15 days, 30 days, or 60 days, or 90 days or 120 days or 180 days or 240 days. The initial rapid release can be, for example, an initial “burst” of release within 1 hour of administration the TKI/PLGA nanoparticle formulation. The initial rapid release can be within the first 24 hours post-administration. In some embodiments, the TKI/PLGA formulation is provided wherein formulation may or may not exhibit an initial rapid release within the first 24 hours post-administration. In some embodiments, the TKI/PLGA formulation is provided wherein formulation may or may not exhibit an initial “burst” of rapid release within 1 hour post-administration. It should be noted that when TKI levels are measured in vitro, an initial rapid release or burst of TKI release from the TKI/PLGA nanoparticle formulation can be seen, but this initial rapid release or burst may or may not be seen in vivo.

In other aspects, for example intranasal, intraocular, intraarticular, inhaled, swallowed administrations, a TKI/PLGA particle or TKI particle formulation provides at least one day, at least two days, at least three days, preferably seven days, including up to and beyond 14 days, or 30 days, 60 days or 90 days of a sustained, steady state release of TKI. In one aspect, a TKI/PLGA particle or TKI particle formulation is provided wherein the TKI/PLGA or TKI particle formulations provide at least one day, at least two days, at least three days, preferably seven days, including up to and beyond 2 weeks days, or 30 days, 60 days or 90 days of a sustained, steady state release of TKI at a rate that does not have adverse effects. The duration of the release of TKI from the TKI/PLGA particles or TKI particles can vary in relation to the total number of TKI/PLGA particles contained in a given formulation.

In certain embodiments, the TKI/PLGA particle, or pharmaceutical composition comprising a plurality of sustained release particles, provides about 2.5% TKI release per week, about 5% TKI release per week, about 10% TKI release per week, about 15% TKI release per week, about 20% TKI release per week, about 25% TKI release per week, about 30% TKI release per week, about 35% TKI release per week, about 40% TKI release per week, about 45% TKI release per week, about 50% TKI release per week, about 60% TKI release per week, about 75% TKI release per week, about 80% TKI release per week, about 90% TKI release per week, or about 100% TKI release per week, and provides therapeutically effective levels of TKIs. In certain embodiments, the TKI/PLGA particles or pharmaceutical composition comprises particles with a biomodal particle size distribution.

In some embodiments, the TKI/PLGA particle or TKI particle formulation retains sustained efficacy even after the TKI is no longer resident at the site of administration, for example, nasal, ocular, auricular, pulmonary, esophageal, and/or after the TKI is no longer detected in the systemic circulation. The TKI/PLGA particle or TKI particle formulation retains sustained efficacy even after the TKI/PLGA or TKI microparticle formulation is no longer resident at the site of administration, for example, in the intra-articular space, and/or the released TKI is no longer detected in the systemic circulation. The TKI/PLGA particle formulation retains sustained efficacy even after the TKI/PLGA particle formulation ceases to release therapeutically effective amounts of TKI. For example, in some embodiments, the TKI released by the TKI/PLGA microparticle formulation retains efficacy for at least one day, two days, three days, four days, five days, six days, one week, at least two weeks, at least three weeks, at least four weeks, at least five weeks, at least six weeks, at least seven weeks, at least eight weeks, at least nine weeks, at least twelve weeks, or more than twelve-weeks post-administration. In some embodiments, the TKI released by the TKI/PLGA particle formulation retains efficacy for a time period that is at least 1.1 times as long, at least 1.2 times as long, at least 1.3 times as long, at least 1.4 times as long, at least 1.5 times as long, at least 1.6 times as long, at least 1.7 times as long, at least 1.8 times as long, at least 1.9 times as long, twice as long, at least 2.5 times as long, at least three times as long, at least 5 times as long, at least 10 times as long, at least 50 times as long, at least 100 times as long, or more than 100 times as long as the residency period for the TKI and/or the TKI/PLGA particle formulation.

In some embodiments, a controlled- or sustained-release TKI/PLGA formulation is provided wherein formulation may or may not exhibit an initial rapid release in addition to the sustained, steady state release of TKI for a second length of time of at least two days, three days, four days, five days, six days, on week, two weeks, preferably at least three weeks, including up to and beyond 30 days, or 60 days, or 90 days or 120 days or 180 days or 240 days. The initial rapid release can be, for example, an initial “burst” of release within ½ hour of administration the TKI/PLGA nanoparticle formulation. The initial rapid release can be within the first 24 hours post-administration. In some embodiments, the TKI/PLGA formulation is provided wherein formulation may or may not exhibit an initial rapid release within the first 24 hours post-administration. In some embodiments, the TKI/PLGA formulation is provided wherein formulation may or may not exhibit an initial “burst” of rapid release within 1 hour post-administration. It should be noted that when TKI levels are measured in vitro, an initial rapid release or burst of TKI release from the TKI/PLGA nanoparticle/microparticle formulation can be seen, but this initial rapid release or burst may or may not be seen in vivo.

These TKI/PLGA particle formulations, preparations, and populations thereof, when administered to a patient, exhibit an improved benefit or other therapeutic outcome in the treatment of a disease, for example a joint related disorder, as compared to the administration, for example administration into the intra-articular space of a joint, of an equivalent amount of the TKI absent any microparticle or other type of incorporation, admixture, or encapsulation. The improved benefit can be any of a variety of laboratory or clinical results. For example, administration of a TKI/PLGA particles is considered more successful than administration of TKI absent any TKI absent any microparticle or other type of incorporation, admixture, or encapsulation if, following administration of the TKI/PLGA particles, one or more of the symptoms associated with the disease is alleviated, reduced, inhibited or does not progress to a further, i.e., worse, state, to a greater extent than the level that is observed after administration of TKI absent any microparticle or other type of incorporation, admixture, or encapsulation. Administration of a TKI/PLGA particles is considered more successful than administration of TKI absent any microparticle or other type of incorporation, admixture, or encapsulation if, following administration of the TKI/PLGA particles, anti-inflammatory activity is sustained for a longer period than the level that is observed after administration of TKI absent any microparticle or other type of incorporation, admixture, or encapsulation.

In addition to the therapeutic component, the TKI/polymer particles disclosed herein may include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

In addition, the TKI/polymer particles may include a solubility-enhancing component provided in an amount effective to enhance the solubility of the TKI(s) relative to substantially identical TKI/polymer particles without the solubility-enhancing component. For example, a TKI/polymer particle may include a P-cyclodextrin, which is effective in enhancing the solubility of the TKI. The β-cyclodextrin may be provided in an amount from about 0.5% (w/w) to about 25% (w/w) of the TKI/polymer particle formulation. In certain embodiments, the β-cyclodextrin is provided in an amount from about 5% (w/w) to about 15% (w/w) of the formulation. In some situations mixtures of polymer particle formulations may be utilized employing the same or different pharmacological agents. In this way, a cocktail of release profiles, giving a biphasic or triphasic release with a single administration is achieved, where the pattern of release may be greatly varied. The TKI/polymer particle formulations may also have a sigmoidal release profile.

Additionally, release modulators such as those described in U.S. Pat. No. 5,869,079 may be included in the TKI/polymer particle formulations. The amount of release modulator employed will be dependent on the desired release profile, the activity of the modulator, and on the release profile of the TKI in the absence of modulator. Electrolytes such as sodium chloride and potassium chloride may also be included in the TKI/polymer particle formulations. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolves more slowly, slowing the exposure of drug particles, and thereby slowing the rate of drug bioerosion.

### Tyrosine Kinases

Phosphorylation of target proteins by kinases is an important mechanism in signal transduction and for regulating enzyme activity. Tyrosine kinases (TK) are a class of over 100 distinct enzymes that transfer a phosphate group from ATP to a tyrosine residue in a polypeptide (Table 1). Tyrosine kinases phosphorylate signaling, adaptor, enzyme and other polypeptides, causing such polypeptides to transmit signals to activate (or inactive) specific cellular functions and responses. There are two major subtypes of tyrosine kinases, receptor tyrosine kinases and cytoplasmic/non-receptor tyrosine kinases.

LE 1
Tyrosine Kinases: Overview of Cellular Distributions and Cellular Functions

| | | |
|---|---|---|
| [symbol] osine kinase | [symbol] ls expressing kinase | [symbol] lular function |

[symbol]

eptor: [symbol]
GFR family:

| | | |
|---|---|---|
| [symbol] ms | [symbol] ocytes, macrophages, osteoclasts | [symbol] l growth, proliferation, |
| [symbol] GFR | [symbol] oblasts, smooth muscle cells, | differentiation, survival, |
| | | and priming |
| | keratinocytes, [symbol] l cells, | [symbol] l growth, proliferation, |
| | chondrocytes | differentiation and survival |
| [symbol] GFR | [symbol] oblasts, smooth muscle cells, | [symbol] l growth, proliferation, |
| | keratinocytes, [symbol] l cells, | differentiation and survival |
| | chondrocytes | |
| [symbol] it | [symbol] matopoietic | [symbol] l growth, proliferation, |
| | progenitor cells, mast | differentiation and survival |
| | cells, [symbol] ordial germ cells, | |
| | interstitial cells of Cajal | |
| [symbol] 3 | [symbol] matopoietic progenitor cells | [symbol] l growth, proliferation, |
| | | differentiation and survival |

[symbol]

GFR family:

| | | |
|---|---|---|
| [symbol] GFR1 | [symbol] ocytes, macrophages, endothelial | [symbol] ocyte and macrophage |
| | cells | migration; vascular permeability |
| [symbol] GFR2 | [symbol] othelial cells | [symbol] culogenesis; angiogenesis |
| [symbol] GFR3 | [symbol] phatic endothelial cells | [symbol] culogenesis; lymphangiogenesis |
| [symbol] R family: | [symbol] oblasts and other mesenchymal | [symbol] ue repair, wound healing, |
| | cells | angiogenesis |

[symbol]

-receptor (cytoplasmic):

| | | |
|---|---|---|
| [symbol] family: | [symbol] quitous | [symbol] l proliferation, survival, |
| | | cell adhesion and migration |
| [symbol] family: | [symbol] ells, Mast cells | [symbol] l proliferation, survival, |
| | | cell adhesion and migration |

[symbol]

family:

| | | |
|---|---|---|
| [symbol] 1 | [symbol] quitous | [symbol] okine signaling |
| [symbol] 2 | [symbol] quitous | [symbol] mone-like cytokine signaling |
| [symbol] 3 | [symbol] lls, B cells, NK cells, myeloid | [symbol] mon-gamma chain cytokine |
| | cells | signaling |
| [symbol] 2 | quitous | [symbol] okine signaling |

[symbol]

C-A family:

| | | |
|---|---|---|
| [symbol] R | [symbol] loid cells (monocytes, | [symbol] minal differentiation |
| | macrophages, granulocytes) | |
| [symbol] | [symbol] quitous | [symbol] l growth; T cell receptor, |
| | | regulation of brain function, |
| | | and adhesion-mediated signaling |
| [symbol] | [symbol] quitous | [symbol] l development, growth, |
| | | replication, adhesion, motility |
| | | [symbol] ntaining tight junctions; |
| | | transmigration of |
| [symbol] | [symbol] quitous | IgA across |
| | | [symbol] helial cells |

[symbol]

C-B family:

| | | |
|---|---|---|
| [symbol] | [symbol] ells, thymocytes | cell proliferation and |
| | | differentiation; thymopoiesis |
| [symbol] K | [symbol] loid cells, lymphoid | liferation, differentiation, |
| | cells | migration |
| [symbol] | [symbol] lls, NK cells | [symbol] ell activation; KIR activation |
| [symbol] | [symbol] loid cells, B cells, mast | R signaling; FceR1 signaling |
| | cells | |

[symbol]

family:

| | | |
|---|---|---|
| [symbol] | [symbol] quitous | liferation, differentiation, |
| | | phagocytosis; tumor suppressor |
| [symbol] 70 | [symbol] lls, NK cells | [symbol] ell activation; KIR activation |

[symbol]

indicates data missing or illegible when filed

Examples of small molecule tyrosine kinase inhibitors are listed in Table 2.

[symbol]

le 2
Examples of Tyrosine Kinase Inhibitors (TKIs)

| | | | | | [symbol] | |
|---|---|---|---|---|---|---|
| | | | | | approval | |
| [symbol] e | de name | ective Target | 0 (nM/L) | [symbol] pany | year | [symbol] approved for |
| inib | [symbol] rif | [symbol] 2, EGFR | 14 | hringer | 3 | -small lung cancer |
| | | | | Ingelheim | | |
| | | | | Pharmac | | |
| / | | | | euticals | | |
| [symbol] itinib | [symbol] ensa | [symbol] | | [symbol] entech | 5 | -small lung cancer |
| inib | [symbol] a | PDGRB, | [symbol] | er | 2 | anced renal cell |
| | | VEGFR1/2/3 | | | | carcinoma |
| [symbol] ozatinib | [symbol] etriq | 3, KIT, | | lixis | 2 | gressive metastatic |
| | | MET, RET, | | | | medullary thyroid |
| | | VEGFR2 | | | | cancer |
| itinib | adia | [symbol] | [symbol] | artis | 4 | -small lung cancer |
| otinib | [symbol] ori | [symbol] , MET | | er | 3 | -small lung cancer |
| [symbol] rafenib | nlar | [symbol] F | | [symbol] oSmithKli | 3 | astatic melanoma |
| | | | | ne | | with BRAF |
| | | | | | | V600E mutation |
| atinib | [symbol] cel | [symbol] -ABL, | | tol Myers | 6 | [symbol] trointestinal |
| | | SRC, KIT, | | | | stromal tumors, |
| | | PDGFRs, | | | | advanced |
| | | EPH, CSK, | | | | renal cell |
| | | DDRs | | | | carcinoma |
| tinib | [symbol] eva | | | Pharms | 4 | -small lung cancer |
| itinib | sa | [symbol] R | | aZeneca | 3 | -small lung cancer |
| tinib | ruvica | | | rmacyclics | 3 | ntle cell lymphoma, |
| | | | | | | chronic |
| | | | | | | lymphocytic |
| | | | | | | leukemia, |
| | | | | | | Waldenstrom |
| | | | | | | macroglobulinemia |
| tinib | evec | [symbol] , KIT, | 0.1, 0.1 | artis | 1 | onic myeloid |
| | | PDGFRs, | | | | leukemia, |
| | | DDRs | | | | gastrointestinal |
| | | | | | | stromal tumors |
| atinib | erb | [symbol] 2, EGFR | 10.8 | [symbol] oSmithKli | 7 | [symbol] 2 + breast cancer |
| | | | | ne | | |
| tinib | igna | [symbol] , KIT, | | artis | 7 | onic myeloid |
| | | PDGFRs, | | | | leukemia |
| | | DDRs | | | | |
| [symbol] ertinib | risso | [symbol] R | | aZeneca | 5 | -small lung cancer |
| opanib | ient | frs, vegfr, | 0 | [symbol] oSmithKli | 2 | anced soft tissue |
| | | KIT | | ne | | sarcoma |
| orafenib | arga | [symbol] pdgfrs, | | er | 3 | anced |
| | | ret, kit, | | | | gastrointestinal |
| | | B-RAF | | | | stromal tumors |
| olitinib | afi | [symbol] 2, JAK1 | 3.3 | te | 4 | [symbol] cythemia vera |
| afenib | avar | [symbol] FRs, | 0 | er | 5 | anced renal cell |
| | | PDGFRs, | | | | carcinoma |
| | | B-RAF, | | | | |
| | | MEK, ERK, | | | | |
| | | c-FMS | | | | |
| itinib | [symbol] nt | [symbol] FRS2, | 0 | er | 6 | [symbol] trointestinal |
| | | PDGFRB, | | | | stromal tumors, |
| | | KIT, RET, | | | | advanced |
| | | CSF1R, | | | | renal cell |
| | | FLT3 | | | | carcinoma |
| [symbol] citinb | anz | [symbol] 3, JAK2, | 0,112 | er | 2 | [symbol] erate to Severe |
| | | JAK1 | | | | Rheumatoid |
| | | | | | | Arthritis |
| acitinib | | [symbol] 1, JAK2 | | te, Lilly | | umatoid Arthritis |
| detanib | [symbol] relsa | , vegfrs, ret, | 0 | aZeneca | 1 | [symbol] ullary thyroid |
| | | tie2, fgfr1 | | | | cancer |

[symbol]

indicates data missing or illegible when filed

### Mast Cell-Mediated Inflammatory Joint Diseases

### Osteoarthritis

Osteoarthritis (OA) is a painful condition caused by a gradual breakdown of joints, loss of cartilage from the joints and joint inflammation. The management of OA includes a combination of non-pharmacologic approaches, such as exercise and patient education; pharmacologic therapies, including oral, topical, and intraarticular medications; and surgical interventions, including total joint arthroplasty. The goal of finding disease-modifying agents for OA is being addressed through ongoing research. Screening for OA in asymptomatic individuals has not become standard of care since there is not preventive, curative, or slowing medication on the market. When humans develop pain in weight bearing joint classically harboring OA or non-classic joints in at risk individuals with convincing history, an X-ray of the affected joint is ordered to evaluate the presence of degenerative disease. Many factors increase the risk of OA including joint injury, joint surgery, degenerative meniscal tears, degeneration of articular cartilage, anterior cruciate ligament tears, collagen and other matrix protein defects, genetic predisposition, body weight and other factors. Humans in the process of developing OA or with features of early OA can be treated with TKI/polymer to prevent the development and progression of OA. Further, humans with established OA as assessed by radiographic evidence can be treated with TKI/polymer to treat pain and tissue/articular damage, slow progression, prevent further progression, and reverse the degenerative process. Further, humans at risk for OA, with early OA, or with established OA can be further tested for the presence of inflammation in the involved joint to identify individuals most likely to respond to treatment with TKI/polymer. Testing for joint inflammation can be performed with imaging markers, such as MRI with or without gadolinium contrast, or an ultrasound, to determine if one or more of the following features indicative of inflammation are present: synovial enhancement or proliferation, an effusion is present, and bone marrow edema. Molecular markers of inflammation can also be tested for, including one or more of CRP, ESR and inflammatory cytokines. If an effusion is present, a joint aspiration can be done and the fluid analyzed for specific inflammatory markers. Finally, clinical history and exam can be used to assess inflammation—including the presence of an effusion on physical exam or morning stiffness on history.

Current osteoarthritis therapy: The treatment of OA is directed towards reduction of symptoms and the prevention of disability. There are no pharmacologic therapies that have been proven to prevent the progression of joint damage due to OA. The goals of therapy for patients with osteoarthritis (OA) are to control pain and swelling, minimize disability, improve the quality of life, and educate the patient about their role in disease management. Pain and other symptoms of OA can be confused with soft tissue processes such as bursitis at periarticular sites; in addition, pain in a particular area may be referred from OA at other site or may be due to a non-articular process. Thus, an important first step in management is to be confident that pain in a particular joint is most likely due to OA at that site. The analgesic acetaminophen is the first line treatment for OA. However, a 2015 review of studies found acetaminophen to only have a small short term benefit. For mild to moderate symptoms effectiveness is similar to non-steroidal anti-inflammatory drugs (NSAIDs), though for more severe symptoms NSAIDs may be more effective. NSAIDs such as naproxen can reduce pain but is associated with greater side effects such as gastrointestinal bleeding. Another class of NSAIDs, COX-2 selective inhibitors (such as celecoxib) are equally effective to NSAIDs with lower rates of adverse gastrointestinal effects but higher rates of cardiovascular disease such as myocardial infarction. Oral opioids, including both weak opioids such as tramadol and stronger opioids such as codeine, are also often prescribed. Oral steroids are not recommended in the treatment of OA. Joint injections of glucocorticoids (such as hydrocortisone) leads to short term pain relief that may last between a few weeks and a few months. Injections of hyaluronic acid have not been found to provide substantial improvement compared to placebo when the knee joint is affected. Once OA joint deterioration becomes intolerable either due to pain or lack of mobility, surgical joint replacement becomes the mainstay of therapy. If problems are significant and more conservative management is ineffective, joint replacement surgery or resurfacing may provide benefit.

It is important to note that multiple therapies that are FDA-approved for rheumatoid arthritis (based on providing significant benefit in RA) have failed to provide or demonstrated only minimal benefit in OA. Examples include hydroxychloroquine (European League Against Rheumatism (EULAR) Congress 2015: Abstract OP0304. Presented Jun. 13, 2015), anti-TNF (infliximab, adalimumab, etanercept) (Magnano, M D et al. (2007). A pilot study of tumor necrosis factor inhibition in erosive/inflammatory osteoarthritis of the hands. The Journal of Rheumatology, 34(6), 1323-1327. PMID:17516620), and anti-IL-1 (IL-1 receptor antagonist; Chevalier X et al. Results from a double blind, placebo-controlled, multicenter trial of a single intraarticular injection of anakinra (Kineret) in patients with osteoarthritis of the knee. 2005 ACR/ARHP Annual Scientific Meeting; Nov. 12-17, 2005; San Diego. Abstract 1339).

Humans in the process of developing OA or with features of early OA can be treated with TKI/polymer to prevent the development and progression of OA. Further, humans with established OA as assessed by radiographic evidence can be treated with TKI/polymer to treat pain and tissue/articular damage, slow progression, prevent further progression, and reverse the degenerative process. Further, humans at risk for OA, with early OA, or with established OA can be further tested for the presence of inflammation in the involved joint to identify individuals most likely to respond to treatment with TKI/polymer. Testing for joint inflammation can be performed with imaging markers, such as MRI with or without gadolinium contrast, or an ultrasound, to determine if one or more of the following features indicative of inflammation are present: synovial enhancement or proliferation, an effusion is present, and bone marrow edema. Molecular markers of inflammation can also be tested for, including one or more of CRP, ESR and inflammatory cytokines. If an effusion is present, a joint aspiration can be done and the fluid analyzed for specific inflammatory markers. Finally, clinical history and exam can be used to assess inflammation—including the presence of an effusion on physical exam or morning stiffness on history.

### Crystal-Induced Arthritis

### Gouty Arthritis

Gout is a painful and potentially debilitating joint disease that develops in some people who have chronically high blood levels of urate (commonly referred to as uric acid). Not everyone with high blood urate levels (called hyperuricemia) develops gout; up to two-thirds of individuals with hyperuricemia never develop symptoms. It is unclear why some people with hyperuricemia develop gout while others do not, but the symptoms of gout result from the body's reaction to deposits of urate crystals in tissues. There are three main phases of gout: acute gouty arthritis, intercritical gout, and chronic tophaceous gout. (1) Acute gouty arthritis—Initial gout flares usually involve a single joint, most often the big toe or knee. This attack is known as acute gouty arthritis. Over time, the attacks can begin to involve multiple joints at once and may be accompanied by fever. People with osteoarthritis in the fingers may experience their first gout attacks in the fingers rather than the toes or knees. (2) Intercritical period—The time between gout attacks is known as an intercritical period. A second attack typically occurs within two years, and additional attacks may occur thereafter. If gout is untreated over a period of several years, the time between attacks may shorten, and attacks may become increasingly severe and prolonged and involve multiple joints. (3) Chronic tophaceous gout—People who have repeated attacks of gout or persistent hyperuricemia for many years can develop tophaceous gout. This designation describes the accumulation of large numbers of urate crystals in masses called tophi. People with this form of gout develop tophi in joints, bursae (the fluid-filled sacs that cushion and protect tissues), bones, and cartilage, or under the skin. Tophi may cause erosion of the bone and eventually joint damage and deformity (called gouty arthropathy). The presence of tophi near the knuckles or small joints of the fingers can be a distressing cosmetic problem. Tophi are usually not painful or tender. However, they can become inflamed and can cause symptoms like those of an acute gouty attack.

The joint at the base of the big toe is the most commonly affected (podagra). It may also present as tophi, kidney stones, or urate nephropathy. It is caused by elevated levels of uric acid in the body. The uric acid crystallizes, and the crystals deposit in joints, tendons, and surrounding tissues. Clinical diagnosis may be confirmed by seeing the characteristic crystals in joint fluid.

The goal of treatment of flares of gouty arthritis is to reduce pain, inflammation, and disability quickly and safely. Deciding which medication to use is based upon several factors, including a person's risk of bleeding, kidney health, and whether there is a past history of an ulcer in the stomach or small intestine. Anti-inflammatory medications are the best treatment for acute gout attacks and are best started early in the course of an attack. People with a history of gout should keep medication on hand to treat an attack because early treatment is an important factor in determining how long it takes to decrease the pain, severity, and duration of an attack. Treatment with nonsteroidal anti-inflammatory drugs (NSAIDs), steroids, or colchicine improves symptoms. Once the acute attack subsides, levels of uric acid are usually lowered via lifestyle changes, and in those with frequent attacks, allopurinol or probenecid provides long-term prevention. The initial aim of treatment is to settle the symptoms of an acute attack. Repeated attacks can be prevented by different drugs used to reduce the serum uric acid levels. Options for acute treatment include nonsteroidal anti-inflammatory drugs (NSAIDs), colchicine, and steroids, while options for prevention include allopurinol, febuxostat, and probenecid.

### Pseudogout Arthritis

Pseudogout arthritis (Pseudogout) is a crystal-induced arthritis (joint disease) induced by the deposition of Calcium pyrophosphate dihydrate (CPPD) crystals in connective tissues. CPPD is an umbrella term for the various clinical subsets, whose naming reflects an emphasis on particular features. For example pseudogout refers to the acute symptoms of joint inflammation or synovitis: red, tender, and swollen joints that may resemble gouty arthritis (a similar condition in which monosodium urate crystals are deposited within the joints). The Ryan and McCarty diagnostic criteria for definite CPPD include observation of positively birefringent rhomboid-shaped crystals in synovial fluids of affected joints, in addition to the presence of radiographic chondrocalcinosis. Some people, particularly older adults, have CPPD crystals in their joints (chondrocalcinosis) but never experience symptoms of pseudogout. Up to 50 percent of people age 90 have chondrocalcinosis. In addition to older age, there are several other factors that increase the risk of accumulating CPPD crystals in the joints, including: (1) Joint trauma—People who have previously experienced a significant injury to or surgery on a joint have an increased risk of developing CPPD crystal deposits. (2) Genetics—People can inherit a predisposition to CPPD crystal deposition (called “familial chondrocalcinosis”); these people are more likely to develop pseudogout or other features of calcium pyrophosphate crystal deposition (CPPD) disease earlier in life. (3) Excess iron—People with a genetic disorder called hemochromatosis, which causes the body to store excess iron, are at an increased risk of developing CPPD crystal deposits.

There is no treatment that can completely remove or prevent the formation of calcium pyrophosphate dihydrate (CPPD) crystals. However, the joint pain and swelling generally resolve with treatment. However, any medication that could reduce the inflammation of chondrocalcinosis bears a risk of causing organ damage, treatment is not advised if the condition is not causing pain. For acute pseudogout, treatments include intraarticular corticosteroid injection, systemic corticosteroids, non-steroidal anti-inflammatory drugs (NSAIDs), or, on occasion, high-dose colchicine. In general, NSAIDs are administered in low doses to help prevent chondrocalcinosis. However, if an acute attack is already occurring, higher doses are administered. Research into surgical removal of calcifications is underway, however this still remains an experimental procedure. For patients who experience frequent episodes of pseudogout, a healthcare provider may prescribe daily colchicine. Use of this medication, which is also often used to treat or prevent gout, can reduce the number of pseudogout attacks.

### Calcific Tendonitis

Calcific tendinitis (also calcific/calcifying/calcified/calcareous tendinitis/tendonitis/tendinopathy, tendinosis calcarea, hydroxyapatite deposition disease (HADD) and calcific periarthritis) is a crystal-induced arthritis (joint disease) which is a form of tendinitis, is a disorder characterized by deposits of hydroxyapatite (a crystalline calcium phosphate) in any tendon of the body, but most commonly in the tendons of the rotator cuff (shoulder), causing pain and inflammation. The condition is related to and may cause adhesive capsulitis (“frozen shoulder”). The calcific deposits are visible on X-ray as discrete lumps or cloudy areas. The deposits look cloudy on X-ray if they are in the process of reabsorption, and this is also when they cause the most pain. The deposits are crystalline when in their resting phase and like toothpaste in the reabsorptive phase. However, poor correlation exists between the appearance of a calcific deposit on plain X-rays and its consistency on needling. Ultrasound is also useful to depict calcific deposits and closely correlates with the stage of disease.

### Apatite-Associated Arthritis

Apatite-associated destructive arthritis (joint disease) includes but is not limited to Milwaukee shoulder syndrome. It is a rheumatological condition similar to calcium pyrophosphate dihydrate deposition disease (CPPD). It is associated with periarticular or intraarticular deposition of hydroxyapatite crystals. Crystal deposition in the joint causes the release of collagenases, serine proteases, elastases, and interleukin-1. This precipitates acute and rapid decline in joint function and degradation of joint anatomy. Subsequently disruption of the rotator cuff ensues. Along with symptomatology, the disease typically presents with positive radiologic findings, often showing marked erosion of the humeral head, cartilage, capsule, and bursae. Though rare, it is most often seen in females beginning in their 50s or 60s. Diagnosis is made with arthrocentesis and Alizarin Red staining along with clinical symptoms.

### Autoimmune Arthritis

### Rheumatoid Arthritis

Rheumatoid arthritis (RA) is an autoimmune disease (joint disease) involving the synovial joints. It is caused by an autoimmune response that causes synovitis and joint destruction. Approximately 60% of RA patients produce anti-citrullinated protein antibodies and rheumatoid factor. Mast cells have been demonstrated to play an important role in the pathogenesis of rheumatoid arthritis.

### Mast Cell-Mediated Allergic Diseases

### Allergic Rhinitis

Allergic rhinitis is defined as symptoms of sneezing, nasal pruritus, airflow obstruction, and mostly clear nasal discharge caused by IgE-mediated reactions against inhaled allergens and involving mucosal inflammation driven by type 2 helper T (Th2) cells. When persons are exposed to an allergen against which they are sensitized, cross-linking by the allergen of IgE bound to mucosal mast cells results in nasal symptoms within minutes. This is due to the release of neuroactive and vasoactive substances from mast cells such as histamine and prostaglandin D2. During the next hours, mast cells and other cells produce a wide array of chemokines and cytokines initiating the Th2 inflammation cascade in the nasal mucosa. The consequence is mucosal inflammation with nasal symptoms that can persist for hours after allergen exposure and mucosa that becomes more reactive to the precipitating allergen (priming) as well as to other allergens and to non-allergenic stimuli, such as strong odors and other irritants (nonspecific nasal hyperresponsiveness). Notably mast cells are also increased in people with perennial non-allergic rhinitis. (Wheatley, L. M., & Togias, A. (2015). Allergic rhinitis. N Engl J Med, 372(5), 456-463. DOI: 10.1056/NEJMcp1412282).

Allergens of importance include seasonal pollens and molds, as well as perennial indoor allergens, such as dust mites, pets, pests, and some molds. The frequency of sensitization to inhalant allergens is increasing and is now more than 40% in many populations in the United States and Europe. Allergic rhinitis contributes to missed or unproductive time at work and school as well as sleep problems. The presence of allergic rhinitis (seasonal or perennial) significantly increases the probability of asthma: up to 40% of people with allergic rhinitis have or will have asthma. (Wheatley, L M, & Togias, A. (2015). Allergic rhinitis. N Engl J Med, 372(5), 456-463. DOI: 10.1056/NEJMcp1412282) (Modena, B D. et al. (2016). Emerging concepts: mast cell involvement in allergic diseases. Translational Research. Published online. doi:10.1016/j.trsl.2016.02.011)

Given the increasing incidence and prevalence of AR, there is a need for new AR treatments, as the current ones are often insufficient to prevent, slow, halt or reverse tissue damage, inflammation or control or alleviate symptoms. Available pharmacologic therapy includes oral or intranasal H1 antihistamines or decongestants, intranasal glucocorticoids, leukotriene inhibitors, or allergen immunotherapy. In addition to a lack of adequate relief with use of these medications, they are fraught with side effects. Oral antihistamines have sedative effects, while intranasal use is limited by bitter taste. Oral decongestant increase blood pressure and intranasal decongestants cause rebound congestion and are ineffective after more than three day of use. Intranasal glucocorticoids have an inappropriately delayed onset of action, and lead to thinning of nasal mucosa and nose bleeds. Finally, the inconvenience of weekly injections and life threatening reactions associated with allergen immunotherapy administration limits its use as well. Finally, while trials have shown at least some benefit to current pharmacologic treatment with seasonal AR, perennial symptoms are notoriously refractory. Currently the only available mast cell targeting therapeutic is cromolyn, a mast cell stabilizer used as a nasal spray. It requires use prior to onset of symptoms, which is often unpredictable in AR. Additionally, it requires frequent dosing and is generally considered to be less efficacious than other treatment options. Given the significant role of mast cells in AR and the currently limited use of mast cell stabilizers due to limited half-life and efficacy, it is novel in the art to target mast cells via a new pathway as proposed in this invention, namely inhibiting or blocking tyrosine kinase signaling pathways mediating mast cell development and/or survival and/or migration and/or activation and/or degranulation using long-acting, sustained-release formulations of various tyrosine kinase inhibitors, to prevent, slow, halt, or reverse inflammation, tissue damage, and symptoms in AR, as disclosed in this invention.

### Non-Allergic Rhinitis, Drug Induced Rhinitis, Occupational Rhinitis, Occupational Rhinitis, Food Induced Rhinitis

Non-allergic rhinitis (NAR) manifests as chronic nasal symptoms not caused by allergic processes. NAR (also known as idiopathic rhinitis, vasomotor rhinitis) can occur with or without eosinophils on nasal smear. Symptoms are similar to AR and occur in response to environmental conditions such as changes in temperature or relative humidity, odors, passive tobacco smoke, alcohol, sexual arousal and emotional factors. Histologic findings are similar to AR including the presence of mast cells. Drug induced rhinitis is caused by oral or topical medications. It has been implicated in use with angiotensin-converting enzyme inhibitors, b-blockers, antihypertensive medications, aspirin, other NSAIDS and oral contraceptives. Often the medication is imperative for the underlying medical condition and the person is forced to cope with the rhinitis/congestion symptoms given the necessity for treatment of the other condition. Occupational rhinitis is triggered by protein and chemical allergens and/or is caused by respiratory sensitizers. In affected persons, intense symptoms lead to suboptimal work production or need to change employment. Food induced rhinitis occurs in response to alcohol or hot/spicy foods and leads to avoidance of certain foods in social situations. (Dykewicz, M. S., & Hamilos, D. L. (2010). Rhinitis and sinusitis. Journal of Allergy and Clinical Immunology, 125(2), S103-S115 doi:10.1016/j.jaci.2009.12.989).

Pharmacologic treatment options for non-allergic and other listed rhinitis conditions are similar to those for AR. Unfortunately they have the same side effect profiles and are even less effective or completely ineffective for these conditions. Given the role of mast cells in NAR and the currently limited use of mast cell stabilizers due to limited half-life and efficacy, it is novel in the art to target mast cells via a new pathway as proposed by this invention to inhibit or block tyrosine kinase signaling pathways mediating mast cell development and/or survival and/or migration and/or activation and/or degranulation using long-acting, sustained-release formulations of various tyrosine kinase inhibitors such as intranasal TKI/polymer particle administration to prevent, slow, halt, or reverse inflammation, tissue damage, and symptoms in NAR, as disclosed in this invention.

### Chronic Rhinosinusitis

Chronic rhinosinusitis (CRS) is defined as an inflammatory condition involving the paranasal sinuses and nasal passages with a minimum duration of 8-12 weeks despite attempts at medical management. Symptoms include nasal obstruction, nasal drainage, facial pain/pressure, and a decreased sense of smell. Two or more symptoms are required as well as objective evidence of mucosal inflammation via confirmation with computed tomography (CT) or magnetic resonance imaging (MRI) (Dykewicz, M. S., & Hamilos, D. L. (2010). Rhinitis and sinusitis. Journal of Allergy and Clinical Immunology, 125(2), S103-S115 doi:10.1016/j.jaci.2009.12.989). CRS is divided into two groups: CRS with nasal polyposis (CRSwNP) and CRS without nasal polyps (CRSsNP). Studies indicate that there exists significant overlap in the inflammatory mechanisms in CRSwNP and CRSsNP. The nasal mucosa demonstrates an increase in infiltrating eosinophils, mast cells, and increased production of IgE and IL-5. CRSwNP can further be divided into two subtypes: individuals with allergic sensitization to environmental aeroallergens (i.e., allergic rhinitis) and those with sensitization to aspirin (i.e., AERD) The tissue inflammatory characteristics of AERD and allergic rhinitis are similar, but underlying mechanisms of disease clearly differ.

Treatment options for CRS are sparse. Surgical intervention and debulking (especially in those with polyposis) and intranasal corticosteroids are the currently available options, but offer limited efficacy, and intranasal corticosteroids are not helpful to prevent recurrence of polyposis. Decongestants or antihistamines are options for patients with comorbid AR. Short courses of oral steroids are another option, but do not lead to sustained efficacy and have side effects including hyperglycemia, bone loss, and mood changes. Given the role of mast cells in CRS and the currently limited use of mast cell stabilizers due to limited half-life and efficacy, it is novel in the art to target mast cells via a new pathway as proposed by this invention to inhibit or block tyrosine kinase signaling pathways mediating mast cell development and/or survival and/or migration and/or activation and/or degranulation using long-acting, sustained-release formulations of various tyrosine kinase inhibitors such as intranasal TKI/polymer particle administration to prevent, slow, halt, or reverse inflammation, tissue damage, and symptoms in CRS, as disclosed in this invention

### Nasal Polyps

Nasal polyps are benign inflammatory growths that arise from inflamed mucosa lining the paranasal sinuses. They can causes invariant unilateral or bilateral nasal obstruction and loss of smell or rhinorrhea as noted above in the CRS section. Presence of polyps is associated with asthma and AERD.

There is strong evidence that mast cells contribute to the formation of nasal polyps and are found in increased numbers in patients with nasal polyps irrespective of allergic sensitization. Polyp tissue from AERD patients has been shown to contain both degranulated mast cells and eosinophils. Nasal lavage in patients with nasal polyps consistently demonstrates increased levels of mast cell granule products as well as eosinophil chemoattractant factors IL-5 and eotaxin. Nasal polyps consist of edematous and fibrotic stroma surrounded by a thickened basement membrane and epithelial cell layer. Eosinophils represent more than 60% of the cellular population and are particularly prevalent between the epithelial cells and thickened basement membrane. As mentioned, the mast cell production of two potent eosinophil chemokines, eosinophil chemoattractant factors IL-5 and eotaxin, is one mechanism in which mast cells trigger eosinophilic tissue infiltration. The accumulation and activation of eosinophils in the mucosal tissue under the influence of IL-5 and eotaxin has been proposed to be the first step in polyp formation. Consistent with its edematous and fibromatous structure, nasal polyps also demonstrate high rates of tissue remodeling and extracellular matrix breakdown. Nasal polyps consistently demonstrate elevated levels of matrix metalloproteinases (MMPs), and MMP-9 appears to be of particular importance. Mast cells are known producers of MMP-9, and its production is further upregulated by the cellular release of tryptase and chymase. Mast cells are also known to secrete TGFb, an important cytokine that causes fibroblastic proliferation, collagen production, and nasal polyp fibroblast expression of vascular endothelial growth factor. Mast cells stimulate nasal polyp epithelial cells and fibroblasts to release other inflammatory factors and chemokines such as granulocyte macrophage colony stimulating factor, thymus- and activation-regulated chemokines, and SCF. Taken together, these findings support the idea that the mast cells are the agent provocateur in both eosinophilic activation and tissue remodeling in nasal polyps (Modena, B D. et al. (2016). Emerging concepts: mast cell involvement in allergic diseases. Translational Research. Published online. doi:10.1016/j.trsl.2016.02.011).

One current treatment option of polyposis includes oral corticosteroids to shrink the polyps, however the effects are temporary and lead to the aforementioned side effects. Intranasal steroids are also recommended to decrease polyp burden but have limited efficacy. Many people with polyposis need surgical removal of polyps, which is often followed by oral or intranasal steroid administration for prevention of recurrence; however polyps tend to still recur. Patients who also have AERD can have improvement of polyposis with aspirin desensitization however desensitization can be life threatening (Dykewicz, M. S., & Hamilos, D. L. (2010). Rhinitis and sinusitis. Journal of Allergy and Clinical Immunology, 125(2), S103-S115 doi:10.1016/j.jaci.2009.12.989). Despite the pathogenic role of mast cells in polyposis, there is a lack of treatment targeting mast cells as they pertain to the growth, shrinkage and treatment of nasal polyps. It is novel in the art to target mast cells via inhibiting or blocking tyrosine kinase signaling pathways mediating mast cell development and/or survival and/or migration and/or activation and/or degranulation using long-acting, sustained-release formulations of various tyrosine kinase inhibitors such as intranasal TKI/polymer particle administration to prevent, slow, halt, reverse and treat nasal polyposis, as disclosed in this invention.

### Asthma Disorders

Asthma is diagnosed through a combination of clinical symptoms (most commonly episodic cough, wheezing, or dyspnea) provoked by typical triggers and physiologic abnormalities. However, the physiologic definition of asthma is relatively nonspecific, consisting of airway hyper-reactivity and airflow limitation during expiration, which is variable and/or reversible with bronchodilators. In most asthma patients, the presence of bronchial hyper-reactivity is never objectively confirmed. Asthma can be triggered by exercise, cold air, viral infections, and exposure to inhaled allergens. Intrinsic abnormalities in airway smooth muscle function, airway remodeling in response to injury or inflammation, and interactions between epithelial and mesenchymal cells appear to modulate and add to the effects of airway inflammation in creating the clinical presentation of asthma. Airway biopsies obtained by bronchoscopy have demonstrated that inflammation in asthma generally involves the same cells that play prominent roles in the allergic response in the nasal passages and skin, whether the individual is atopic or not. This supports the belief that the consequences of mast cell activation, mediated by a variety of cells, cytokines, and other mediators, are key to the development of clinical asthma. Mast cells are increased in number in asthmatic airways and may be found in close association with airway smooth muscle cells. In addition to producing bronchoconstricting mediators (e.g., histamine, certain prostaglandins, and leukotrienes), mast cells also store and release tumor necrosis factor (TNF)-alpha, which is important in the recruitment and activation of inflammatory cells and in altered function and growth of airway smooth muscle.

There are various types of asthma including atopic/allergic and non-atopic phenotypes (including but not limited to exercise-induced, nocturnal, occupational, steroid-resistant, cough variant, medication induced, obesity related, childhood vs adult onset, eosinophilic, aspirin exacerbated respiratory disease (AERD), premenopausal, asthmatic granulomatosis). Asthma treatment is based on severity which includes the four categories of intermittent, mild, persistent, moderate persistent and severe persistent. (National Asthma Education and Prevention Program: Expert panel report II: Guidelines for the diagnosis and management of asthma. National Heart, Lung, and Blood Institute (NIH publication no. 97-4051, Bethesda, Md. 1997) (Moore W C, et al. (2010) Identification of asthma phenotypes using cluster analysis in the Severe Asthma Research Program. Am J Respir Crit Care Med; 181:315. PMID: 19892860) (Brightling C E et al. (2002) Mast-cell infiltration of airway smooth muscle in asthma. N Engl J Med; 346:1699. PMID 12037149) (Nakae S et al. (2007) Mast cell-derived TNF contributes to airway hyperreactivity, inflammation, and TH2 cytokine production in an asthma model in mice. J Allergy Clin Immunol; 120:48.PMID 17482668).

Pharmacologic treatment is the mainstay of management in most patients with asthma. The 2007 National Asthma Education and Prevention Program (NAEPP) Expert Panel Report presented a stepwise approach to pharmacologic therapy in varying combinations of short acting bronchodilators, long acting bronchodilators, low to high doses of inhaled glucocorticoids, cromolyn, leukotriene antagonists, or theophylline. Given the refractory nature of asthma and limited effectiveness of current treatment options for some people, several novel treatments are under investigation including monoclonal antibodies to IgE (omalizumab) and IL-5 (mepolizumab) (National Asthma Education and Prevention Program: Expert panel report III: Guidelines for the diagnosis and management of asthma. Bethesda, Md.: National Heart, Lung, and Blood Institute, 2007. (NIH publication no. 08-4051) (Fanta C H. (2009) Asthma. N Engl J Med; 360:1002. PMID 19264689).

Current asthma treatments have a significant side effect profile including cardiac arrhythmias with bronchodilators and theophylline, anaphylaxis, blood dyscrasias, and rash from cromolyn and leukotriene antagonists, and growth retardation and systemic side effects associated with glucocorticoids. Given the significant role of mast cells in asthma and their limited targeting for therapeutic purpose, it is novel in the art as reported in this invention to target mast cells via inhibiting or blocking tyrosine kinase signaling pathways mediating mast cell development and/or survival and/or migration and/or activation and/or degranulation using long-acting, sustained-release formulations of various tyrosine kinase inhibitors such as inhaled TKI/polymer particle administration, to prevent, slow, halt, reverse and treat inflammation, tissue damage, and symptoms in asthma, as disclosed in this invention.

### Asthma-COPD Overlap and COPD

Many of the features described for asthma overlap with chronic obstructive pulmonary disease (COPD), a common respiratory condition characterized by airflow limitation that is usually progressive and associated with an enhanced chronic inflammatory response in the airways and the lung to noxious particles or gases. Exacerbations and comorbidities contribute to the overall severity in individual patients (Global Strategy for the Diagnosis, Management and Prevention of COPD, Global Initiative for Chronic Obstructive Lung Disease (GOLD) 2016). In COPD, post-bronchodilator pulmonary function testing may confirm little or no reversibility of the airflow obstruction. At other times, however, the distinction is less clear, such as when patients with COPD exhibit episodic symptoms and a large reversible component to their airflow obstruction. Recognition of these overlapping features of both asthma and COPD in some patients has led to description of the asthma-COPD overlap syndrome.

The predominant pathologic changes of chronic obstructive pulmonary disease (COPD) are found in the airways, but changes are also seen in the lung parenchyma and pulmonary vasculature. In an individual, the pattern of pathologic changes depends on the underlying disease (e.g., chronic bronchitis, emphysema, alpha-1 antitrypsin deficiency), possibly individual susceptibility, and disease severity. High resolution CT can assess lung parenchyma, airways, and pulmonary vasculature. In particular two pathological phenotypes of COPD centrilobular (CLE) and panlobular emphysema (PLE) have shown important differences in their overall inflammation with an unexpected protagonism of mast cells, which are related to airway reactivity. These findings highlight the distinctness of these COPD phenotypes and the role of mast cells in the pathophysiology of COPD (Ballarin et al. (2012) Mast cell infiltration discriminates between histopathological phenotypes of chronic obstructive pulmonary disease, American journal of respiratory and critical care medicine, 186(3), 233-239. doi: 10.1164/rccm.201112-21420C). Disease severity dictates COPD treatment, as described by the GOLD severity criteria. Similar to asthma, pharmacologic therapy consists of varying combinations of short acting bronchodilators, long acting bronchodilators, low to high doses of inhaled glucocorticoids and theophylline are used. Additional options include short to long acting anticholinergic agents and phosphodiesterase inhibitors. The fact that COPD is the third-ranked cause of death in the United States, killing more than 120,000 individuals each year and causes high resource utilization with frequent clinician office visits, frequent hospitalizations due to acute exacerbations, and the need for chronic therapy (e.g., supplemental oxygen therapy, medication) speaks to the ineffectiveness of current pharmacotherapy options (Miniño A M et al. (2011) Deaths: final data for 2008. Natl Vital Stat Rep 2011; 59:1. PMID 22808755) (Buist A S et al. (2007) International variation in the prevalence of COPD (the BOLD Study): a population-based prevalence study. Lancet; 370:741. PMID 17765523). Given the role of mast cells in COPD and lack of targeted mast cell treatment in this condition, it is novel in the art to target mast cells via inhibiting or blocking tyrosine kinase signaling pathways mediating mast cell development and/or survival and/or migration and/or activation and/or degranulation using long-acting, sustained-release formulations of various tyrosine kinase inhibitors such as inhaled TKI/polymer particle administration to prevent, slow, halt, reverse and treat inflammation, tissue damage, and symptoms in COPD, as disclosed in this invention, as disclosed in this invention.

### Aspirin-Exacerbated Respiratory Disease (AERD)

Aspirin-exacerbated respiratory disease (AERD) is characterized by asthma, chronic rhinosinusitis with nasal polyposis, and pathognomonic respiratory reactions to aspirin (Samter's triad). It has been estimated that this syndrome affects 7% of adults with asthma and 14% of those who have severe asthma. Pathologically, AERD is characterized by marked eosinophilic inflammation and ongoing mast-cell activation in the respiratory mucosa. The frequent recurrence of nasal polyps after surgery, as well as the requirement for high-dose glucocorticoids to manage the asthma, reflect the aggressive, persistent nature of the disease. The typical onset is in adulthood, with or without preexisting asthma, rhinitis, or atopy.

All nonsteroidal anti-inflammatory drugs (NSAIDs) that inhibit both cyclooxygenase (COX)-1 and COX-2 may provoke the pathognomonic reactions in AERD; these reactions are accompanied by idiosyncratic activation of respiratory tract mast cells. NSAID-induced increases in LTE4 during clinical reactions are paralleled by increases in the products of activated mast cells (histamine, tryptase, and prostaglandin D2 [PGD2]) and are blocked by the administration of mast-cell-stabilizing drugs. Thus, mast-cell activation contributes substantially to cysteinyl leukotriene formation when COX-1 is inhibited in AERD. In contrast, patients with AERD can usually be treated with COX-2-selective drugs without having these reactions. The fact that structurally diverse NSAIDs that block COX-1 all provoke reactions reflects an enigmatic requirement for COX-1-derived prostaglandins to maintain a tenuous homeostasis. Curiously, the reactions also induce a refractory state in which NSAIDs can be used with diminished or no sequelae (desensitization); in fact, after desensitization, high-dose aspirin has therapeutic benefits (Laidlaw, T. M., & Boyce, J. A. (2016). Aspirin-Exacerbated Respiratory Disease—New Prime Suspects. New England Journal of Medicine, 374(5), 484-488. DOI: 10.1056/NEJMcibrl 514013).

Current treatment for AERD is asthma management, polyposis management, and aspirin desensitization, which is the only definitive treatment. Aspirin desensitization is a multiple day process with several days spent in the physician's office. Furthermore, patients need to take high dose aspirin the rest of their lives which can have side effects including bleeding diathesis and gastrointestinal complications. To decrease the incidence of severe life threatening reactions with aspirin desensitization, the administration of leukotriene inhibitors and omalizumab are being investigated peri-desensitization. We propose a new art to use TKI/polymer particles prior to, peri- and after-desensitization in AERD patients. Given the role of mast cells in the disease and lack of targeted treatment, it is novel in the art to target mast cells via inhibiting or blocking tyrosine kinase signaling pathways mediating mast cell development and/or survival and/or migration and/or activation and/or degranulation using long-acting, sustained-release formulations of various tyrosine kinase inhibitors such as intranasal or inhaled TKI/polymer particles to decrease the incidence of life threatening side effects related to desensitization and decrease the amount of aspirin needed to maintain tolerance after desensitization, as disclosed in this invention. As AERD frequently also presents with nasal polyps and asthma, TKI/polymer particles would be helpful to prevent, slow, halt, reverse and treat nasal polyposis and asthma associated with this condition, as disclosed in this invention

The relationship between mast cells and eosinophils is complex. There is in fact a large array of mediator and receptor signaling that occurs between the 2 cell types. These dual interactions act to guide and enhance each other's function. Recently, the totality of this “cross-talk” between eosinophils and mast cells has been given the name “allergic effector unit.” In this way, eosinophilic and mast cell inflammation seen in AERD is similar to allergen driven inflammation as it occurs in the upper and lower airways (i.e., allergic rhinitis and atopic asthma), the skin (i.e., atopic dermatitis), and the esophagus (i.e., eosinophilic esophagitis).

### Eosinophilic Esophaqitis—EOE

When gastrointestinal eosinophilia is limited to the esophagus, is accompanied by characteristic symptoms (dysphagia, food impaction, gastroesophageal reflux disease (GERD)), and other causes of eosinophilia have been ruled out, it is termed eosinophilic esophagitis. A panel of experts defined eosinophilic esophagitis as “a chronic, immune/antigen-mediated, esophageal disease characterized clinically by symptoms related to esophageal dysfunction and histologically by eosinophil-predominant inflammation” (Liacouras C A et al. (2011) Eosinophilic esophagitis: updated consensus recommendations for children and adults. J Allergy Clin Immunol; 128:3. PMID 21477849). Diagnosis is based on symptoms, histology with eosinophil-predominant inflammation on esophageal biopsy, characteristically consisting of a peak value of ≥15 eosinophils per high power field, and persistence after proton pump inhibitor therapy. Classic endoscopic findings include strictures and linear furrows.

Increased numbers of mast cells are seen in esophageal tissue samples from patients with EoE, and degranulation is common. The exact role of mast cells in EoE is unclear. Results from a murine model of EoE suggest that mast cells may play an important role in esophageal remodeling in EoE by promoting muscle cell hyperplasia and hypertrophy. Elevated TGF-beta, produced by eosinophils and mast cells, contributes to esophageal tissue remodeling and smooth muscle dysfunction in patients with EoE, similar to that seen in the airways of patients with asthma, further supporting the link between esophageal and pulmonary inflammation. (Niranjan R et al. (2013) Pathogenic role of mast cells in experimental eosinophilic esophagitis Am J Physiol Gastrointest Liver Physiol; 304:G1087. PMID 23599040) (Aceves S S et al. (2007) Esophageal remodeling in pediatric eosinophilic esophagitis. J Allergy Clin Immunol; 119:206.PMID 17208603).

Current treatment options for EOE include swallowed topical glucocorticoids or systemic glucocorticoids. When structural changes occur in the esophagus, surgical dilation is required. Other tried but ineffective treatments include antihistamines, cromolyn, and anti-TNF therapy. Data about treatment with montelukast, mepolizumab, reslizumab, omalizumab, and anti-TNF therapy is inconclusive. Given the role of mast cells in EOE and the lack of targeted therapeutic directed towards mast cells, it is novel in the art to target mast cells via a new pathway-inhibiting or blocking tyrosine kinase signaling pathways mediating mast cell development and/or survival and/or migration and/or activation and/or degranulation using long-acting, sustained-release formulations of various tyrosine kinase inhibitors such as swallowed TKI/polymer particle administration to targeting the esophagus to prevent, slow, halt, reverse and treat inflammation, tissue damage, and symptoms in EOE, as disclosed in this invention.

### Allergic Conjunctivitis

The umbrella terms “allergic conjunctivitis” or “ocular allergy” are used to describe a heterogeneous array of conjunctival diseases. The initial assumption was that each of these conditions was caused by the IgE-mediated hypersensitivity reaction. In actuality, there exists many other non-IgE-mediated mechanisms involved, such as non-IgE mast cell activation and late-phase reactions. All types of allergic eye disease (seasonal and perennial allergic conjunctivitis, vernal keratoconjunctivitis (VKC) and atopic keratoconjunctivitis (AKC)) have been demonstrated to have higher numbers of conjunctival mast cells, even in the absence of leukocyte infiltration. The acute reaction is predominantly due to mast cell degranulation and therefore typically controlled with directed topical therapy (e.g., olopatadine, ketotifen) against mast cells and their mediators. Mast cell degranulation and activation in the early phase reaction then drives a late-phase reaction. Conversely, VKC and atopic keratoconjunctivitis AKC are chronic, potentially sight-threatening conditions thought to be only partially allergen dependent. Giant papillary conjunctivitis (GPC) is a delayed hypersensitivity reaction secondary to trauma involving contact between a foreign body and the tarsal conjunctiva, or due to an immune reaction to protein deposits on contact lenses, which rub against the eyelid upon each blink.

Treatment of the allergic acute conditions includes topical antihistamines, decongestants, and mast cell stabilizers. Treatment efficacy is mixed and given the sensitivity of the ocular surface, frequently are poorly tolerated. The chronic conditions require immunosuppressive therapy and given significant morbidity, warrant other treatment options as suggested by this patent. Given the role of mast cells in ocular allergy and the currently limited use of mast cell stabilizers due to limited half-life and efficacy, it is novel in the art to target mast cells via a new pathway—inhibiting or blocking tyrosine kinase signaling pathways mediating mast cell development and/or survival and/or migration and/or activation and/or degranulation using long-acting, sustained-release formulations of various tyrosine kinase inhibitors such as intraocular TKI/polymer particle administration to prevent, slow, halt, reverse and treat inflammation, tissue damage, and symptoms in ocular allergy, as disclosed in this invention.

All references, patents, patent applications or other documents cited are hereby incorporated by reference in their entirety for all purposes. These references, patents, patent applications or other documents include: US20050043706, US20110206773, US20120282298, US2014031167, US20090136579, US20140148474 A1, US20100168116, US20140227326A1, U.S. Pat. Nos. 6,297,227B1, 5,869,079, 4,293,539. 7,063,748B2, 6,340,475, US20100168116, U.S. Pat. Nos. 5,702,716, 4,293,539, 7,771,742, 7,906,512, US20130040984, U.S. Pat. No. 8,865,220, US20150023993, U.S. Pat. Nos. 7,713,975, 6,113,947, US20090136579, US20060046961, U.S. Pat. Nos. 5,192,741, 5,744,163, 6,528,097, US20140045895, U.S. Pat. No. 8,673,336, CN101015559, WO2012104402A1, WO1995013799A1, WO2005044354A1, WO1999049923A1. Genovese M C et al. Baricitinib in patients with refractory rheumatoid arthritis (2016) NEJM, 374(13), 1243-1252. PMID: 27028914; Ruzza P et al. (2009) Therapeutic prospect of Syk inhibitors. Expert opinion on therapeutic patents, 19(10), 1361-1376. DOI: 10.1517/13543770903207039; Virk, H et al. (2016). Mast cells and their activation in lung disease. Translational Research. Published online. doi:10.1016/j.trsl.2016.01.005; Guyer B et al. (2004) J Allergy Clin Immunol., 113(2); S28-29. doi:10.1016/j.jaci.2003.12.058; Marslin, G et al. (2015) Delivery as nanoparticles reduces imatinib mesylate-induced cardiotoxicity and improves anticancer activity, International journal of nanomedicine, 10, 3163-3170 doi: 10.2147/IJN.S75962; Benny, O et al. (2009) Local delivery of poly lactic-co-glycolic acid microspheres containing imatinib mesylate inhibits intracranial xenograft glioma growth, Clinical Cancer Research, 15(4), 1222-1231 doi:10.1158/1078-0432; Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90; Horisawa, E et al. (2002). Size-dependency of DL-lactide/glycolide copolymer particulates for intra-articular delivery system on phagocytosis in rat synovium. Pharmaceutical research, 19(2), 132-139 DOI:10.1023/A:1014260513728; Ratcliffe, J H et al. (1984). Preparation and evaluation of biodegradable polymeric systems for the intra-articular delivery of drugs. Journal of pharmacy and pharmacology, 36(7), 431-436. DOI: 10.1111/j.2042-7158.1984.tb04419.x; Kublik H and Vifgren M T. (1998) Nasal delivery systems and their effect on deposition and absorption. Advanced Drug Delivery Review 29; 157-177 PMID:10837586; Djupesland P G. (2013) Nasal drug delivery devices: characteristics and performance in a clinical perspective a review. Drug Deliv and Transl. Res. 3:42-62. DOI 10.1007/s13346-012-0108-9; Majgainya, S et al. (2015) Novel approach for nose-to-brain drug delivery bypassing blood brain barrier by pressurized olfactory delivery device. J App Pharm 7; 148-163. ISSN 19204159; Surber, C et al., (2011). Nasal drug delivery in humans. Curr. Probl. Dermatol. 40; pp 20-35 doi: 10.1159/000321044; Kumar, A et al., (2016). Nasal nanotechnology: revolution for efficient therapeutics delivery. Drug Delivery. 23: pp 681-693 doi: 10.3109/10717544.2014.920431; European League Against Rheumatism (EULAR) Congress 2015: Abstract OP0304. Presented Jun. 13, 2015; Magnano, M D et al. (2007). A pilot study of tumor necrosis factor inhibition in erosive/inflammatory osteoarthritis of the hands. The Journal of Rheumatology, 34(6), 1323-1327. PMID:17516620; IL-1 receptor antagonist; Chevalier X et al. Results from a double blind, placebo-controlled, multicenter trial of a single intraarticular injection of anakinra (Kineret) in patients with osteoarthritis of the knee. 2005 ACR/ARHP Annual Scientific Meeting; Nov. 12-17, 2005; San Diego. Abstract 1339; Wheatley, L. M., & Togias, A. (2015). Allergic rhinitis. N Engl J Med, 372(5), 456-463. DOI: 10.1056/NEJMcp1412282; Modena, B D. et al. (2016). Emerging concepts: mast cell involvement in allergic diseases. Translational Research. Published online. doi:10.1016/j.trsl.2016.02.011; (Dykewicz, M. S., & Hamilos, D. L. (2010). Rhinitis and sinusitis. Journal of Allergy and Clinical Immunology, 125(2), S103-S115 doi:10.1016/j.jaci.2009.12.989; National Asthma Education and Prevention Program: Expert panel report II: Guidelines for the diagnosis and management of asthma. National Heart, Lung, and Blood Institute (NIH publication no. 97-4051, Bethesda, Md. 1997; Moore W C, et al. (2010) Identification of asthma phenotypes using cluster analysis in the Severe Asthma Research Program. Am J Respir Crit Care Med; 181:315. PMID: 19892860; Brightling C E et al. (2002) Mast-cell infiltration of airway smooth muscle in asthma. N Engl J Med; 346:1699. PMID 12037149); Nakae S et al. (2007) Mast cell-derived TNF contributes to airway hyperreactivity, inflammation, and TH2 cytokine production in an asthma model in mice. J Allergy Clin Immunol; 120:48.PMID 17482668; National Asthma Education and Prevention Program: Expert panel report III: Guidelines for the diagnosis and management of asthma. Bethesda, Md.: National Heart, Lung, and Blood Institute, 2007 NIH publication no. 08-4051; Fanta C H. (2009) Asthma. N Engl J Med; 360:1002. PMID 19264689; Global Strategy for the Diagnosis, Management and Prevention of COPD, Global Initiative for Chronic Obstructive Lung Disease (GOLD) 2016; Ballarin et al. (2012) Mast cell infiltration discriminates between histopathological phenotypes of chronic obstructive pulmonary disease, American journal of respiratory and critical care medicine, 186(3), 233-239. doi: 10.1164/rccm.201112-21420C; Miniño A M et al. (2011) Deaths: final data for 2008. Natl Vital Stat Rep 2011; 59:1. PMID 22808755; Buist A S et al. (2007) International variation in the prevalence of COPD (the BOLD Study): a population-based prevalence study. Lancet; 370:741. PMID 17765523; Laidlaw, T. M., & Boyce, J. A. (2016). Aspirin-Exacerbated Respiratory Disease—New Prime Suspects. New England Journal of Medicine, 374(5), 484-488. DOI: 10.1056/NEJMcibr1514013; Liacouras C A et al. (2011) Eosinophilic esophagitis: updated consensus recommendations for children and adults. J Allergy Clin Immunol; 128:3. PMID 21477849; Niranjan R et al. (2013) Pathogenic role of mast cells in experimental eosinophilic esophagitis Am J Physiol Gastrointest Liver Physiol; 304:G1087. PMID 23599040; Aceves S S et al. (2007) Esophageal remodeling in pediatric eosinophilic esophagitis. J Allergy Clin Immunol; 119:206.PMID 17208603; Glasson, S s, et al. (2007) The surgical destabilization of the medical mesicus (DMM) model of osteoarthritis in the 129/SvEv mouse. Osteoarthritis Cartilage, 15; 1061-1069. doi:10.1016/j.joca.2007.03.006; Kamekura, S. et al. (2005) Osteoarthritis development in novel experimental mouse models induced by knee joint instability. Osteoarthritis Cartilage. 13; 632-641. doi:10.1016/j.joca.2005.03.004; Kamekura, S et al. (2005). Osteoarthritis development in novel experimental mouse models induced by knee joint instability. Osteoarthritis and cartilage, 13(7), 632-64. doi:10.1016/j.joca.2005.03.004) (Bendele, A M. (2001) Animal models of osteoarthritis. J Musculoskelet Neuronal Interact, 1(4), 363-376. PMID:15758487; Blom, A B. et al. (2004) Synovial lining macrophages mediate osteophyte formation during experimental osteoarthritis. Osteoarthritis Cartilage. 12; 627-635. doi:10.1016/j.joca.2004.03.003; Marslin, G et al. (2015) Delivery as nanoparticles reduces imatinib mesylate-induced cardiotoxicity and improves anticancer activity. International journal of nanomedicine, 10, 3163-3170 doi: 10.2147/IJN.S75962; Benny, O et al. (2009) Local delivery of poly lactic-co-glycolic acid microspheres containing imatinib mesylate inhibits intracranial xenograft glioma growth, Clinical Cancer Research, 15(4), 1222-1231 doi:10.1158/1078-0432; Chang, D Y et al. (2015). Therapeutic effects of intranasal cyclosporine for eosinophilic rhinosinusitis with nasal polyps in a mouse model. American journal of rhinology & allergy, 29(1), e29-e32 doi: 10.2500/ajra.2015.29.4152; Vlahos, R et al. (2015). (In Press) Preclinical murine models of chronic obstructive pulmonary disease. European Journal of Pharmacology, 1-7 doi: 10.1016/j.ejphar.2015.03.029; Fricker, M et al. (2014). Animal models of chronic obstructive pulmonary disease. Expert opinion on drug discovery, 9(6), 629-645 doi: 10.1517/17460441.2014.909805; Liu, T et al. (2015). Aspirin-Exacerbated Respiratory Disease Involves a Cysteinyl Leukotriene-Driven IL-33-Mediated Mast Cell Activation Pathway. The Journal of Immunology, 195(8), 3537-3545 doi: 10.4049/jimmunol.1500905; Liu, T et al. (2013). Prostaglandin E2 deficiency causes a phenotype of aspirin sensitivity that depends on platelets and cysteinyl leukotrienes. Proceedings of the National Academy of Sciences, 110(42), 16987-16992 doi: 10.1073/pnas. 1313185110.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

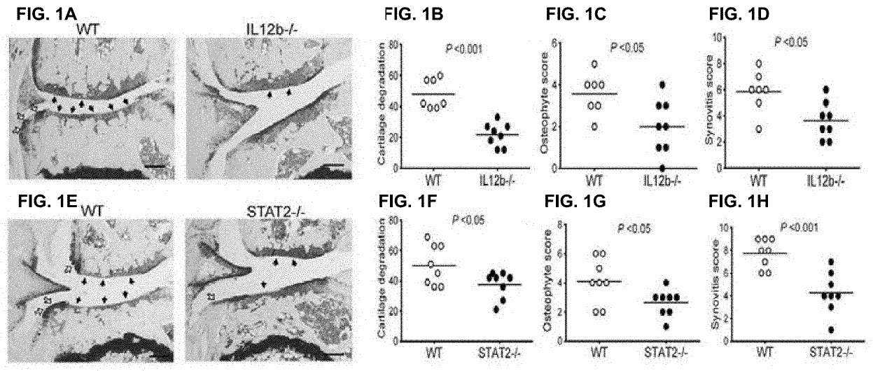

Mouse models of OA. C57BL6 (B6) mice (n=7-10 per group) are surgically induced to develop OA by medial meniscectomy (MM) or destabilization of the medial meniscus (DMM). Experiments were performed under protocols approved by the Stanford University Committee of Animal Research and in accordance with NIH guidelines. Murine OA was generated by surgically either by destabilization of the DMM (Glasson, S s, et al. (2007) The surgical destabilization of the medical meniscus (DMM) model of osteoarthritis in the 129/SvEv mouse. Osteoarthritis Cartilage, 15; 1061-1069. doi:10.1016/j.joca.2007.03.006) or by MM (Kamekura, S. et al. (2005) Osteoarthritis development in novel experimental mouse models induced by knee joint instability. Osteoarthritis Cartilage. 13; 632-641. doi:10.1016/j.joca.2005.03.004). One week and two weeks following surgical induction of the MM or DMM model, the articular cartilage is intact and there is no overt evidence of OA—at this time point the mice walk and run normally and are asymptomatic or can exhibit mild joint symptoms, but due to the surgical procedure the mice are in a pre- or early-OA disease state and progress to develop OA over the following months (FIG. 1A).

Histological scoring of murine OA. Mice were euthanized 8-20 weeks after surgery. Their stifle joints were decalcified in EDTA solution, fixed in 4% paraformaldehyde, and embedded in paraffin. Serial 4 μm sections were cut and stained with toluidine blue. Scoring of arthritis in these histology sections was done according to a modified version of previously described composite scoring systems (Kamekura, S et al. (2005). Osteoarthritis development in novel experimental mouse models induced by knee joint instability. Osteoarthritis and cartilage, 13(7), 632-64. doi:10.1016/j.joca.2005.03.004) (Bendele, A M. (2001) Animal models of osteoarthritis. J Musculoskelet Neuronal Interact, 1(4), 363-376. PMID:15758487). The “Cartilage Degeneration Score” (also termed the “OA Score” or “Histologic Score”) was calculated as follows: cartilage degeneration (0-4) was multiplied by the width (1=1/3, 2=2/3, and 3=3/3 of surface area) of each third of the femoral-medial and tibial-medial condyles, and the scores for the 6 regions were summed. To evaluate osteophyte formation, we scored toluidine-blue-stained sections according to a previously described scoring system (Kamekura, S. et al. (2005) Osteoarthritis development in novel experimental mouse models induced by knee joint instability. Osteoarthritis Cartilage. 13; 632-641. doi:10.1016/j.joca.2005.03.004): 0, none; 1, formation of cartilage-like tissues; 2, increase of cartilaginous matrix; 3, endochondral ossification. To evaluate synovitis, we scored H&E-stained sections according to a previously described scoring system (Blom, A B. et al. (2004) Synovial lining macrophages mediate osteophyte formation during experimental osteoarthritis. Osteoarthritis Cartilage. 12; 627-635. doi:10.1016/j.joca.2004.03.003): 0, no changes compared to normal joints; 1, thickening of the synovial lining and some influx of inflammatory cells; 2, thickening of the synovial lining and intermediate influx of inflammatory cells; and 3, profound thickening of the synovial lining (more than four cell layers) and maximal observed influx of inflammatory cells. Scores for osteophyte formation and synovitis (inflammation in the synovial lining and joint) were recorded for the femoral-medial and the tibial-medial condyles on the operated side of the joint, and the scores for the two regions were summed and statistical comparisons performed using the T test.

Mouse models of allergic asthma. Mouse models of the acute allergic response to inhaled allergens are widely used to elucidate the mechanisms underlying the immunologic and inflammatory responses in asthma, and for the identification and investigation of novel targets for controlling allergic inflammation. The most commonly used strain of mouse for antigen challenge models is BALB/c as they develop T helper cell 2 (Th2)-biased immunological responses. Other strains (C57BL/6 and A/J) are also used in allergen challenge studies. The acutely challenged mouse shows elevated levels of IgE, airway inflammation, goblet cell hyperplasia, epithelial hypertrophy, airway hyper-responsiveness (AHR) to specific stimuli as well as early- and late-phase bronchoconstriction in response to allergen challenge. Chronic allergen exposure in mice results in multiple features of clinical asthma, such as airway remodeling and persistent AHR, are present. Chronic allergen challenge models utilize repeated exposure of the airways to low levels of allergen for periods of up to 12 weeks. Different allergens have been employed and co-administration of an adjuvant is not always required. Ovalbumin (OVA) derived from chicken egg is a frequently used allergen that induces a robust, allergic pulmonary inflammation in laboratory rodents. In addition to OVA, other groups have used alternative allergens for example house dust mite (HDM) and cockroach extracts.

The House Dust Mite (HDM)-induced asthma model in the mouse is used to assess the in vivo efficacy of anti-asthma drugs. This model features many similarities to human allergic asthma, including the presence of eosinophilic lung inflammation and the release of inflammatory mediators and cytokines primarily associated with Th2-type inflammation. Total and differential cell counts of inflammatory cells in the lung are performed on the bronchoalveolar lavage (BAL) fluid at various time points to observe the time course of inflammation and evaluate the effects of compounds.

Measurement of Airway hyperresponsiveness (AHR). AHR to the acetylcholine challenge, defined by the time-integrated rise in peak airway pressure [airway-pressure-time index (APTI) in centimeters of H2O 3 seconds].

Histological assessment of murine asthma. The lungs and trachea are stained with H-E for pathologic alteration, toluidine blue for mast cell, congo red for eosinophil, PAS for goblet cell and Masson's-trichrome for fibrosis.

### Example 1

### Mice Lacking IL-12Beta (IL-12b, II12b) or STAT2, Molecules that Utilize JAK Tyrosine Kinase Signaling Pathways, were Protected from the DMM Model of OA in Mice

Mice genetically deficient for the major inflammatory cytokine IL-12b or the STAT2 transcription factor downstream of IFNgamma (IFNγ) were induced to develop osteoarthritis by surgically-induced destabilization of the medial meniscus (DMM). 20 weeks following surgery, their knee joints were harvested, fixed and processed for histology. Tissue sections were stained by safranin-o to visualize cartilage damage and inflammation. FIGS. 1A-1D are representative knee joint sections and graphs illustrating reduction in osteoarthritis pathologies in mice that lack IL-12b, a major inflammatory cytokine involved in several inflammatory diseases including RA. Mice were induced to develop osteoarthritis by surgically-induced destabilization of the medial meniscus (DMM). FIG. 1B shows the cartilage degradation scores in control or wild-type (WT, open circles) and IL-12b-deficient (IL12b−/−, closed circles), assessed using a semi-quantitative scoring system 20-weeks post DMM surgery. FIG. 1C shows the osteophyte score. FIG. 1D shows the synovitis score for the same mice. Statistical analyses were done by unpaired Student's t test. FIGS. 1E-1H are representative knee joint sections and graphs illustrating reduction in osteoarthritis pathologies in mice that lack STAT2, a transcription factor downstream of IFNγ signaling known to induce macrophage activation in several inflammatory diseases including RA. FIG. 1F shows the cartilage degradation scores in control or wild-type (WT, open circles) and STAT2-deficient (Stat2−/−, closed circles), assessed using a semi-quantitative scoring system 20-weeks post DMM surgery. FIG. 1G shows the osteophyte score. FIG. 1H shows the synovitis score for the same mice. Statistical analyses were performed by unpaired Student's t test.

Thus, we demonstrated that genetic deficiency in IL-12b or STAT2, molecules that trigger or are associated with tyrosine kinase signaling pathways, reduced the severity of osteoarthritis of, and reduced joint inflammation in, mice induced to develop OA via DMM.

### Example 2

### Mice Deficient in Fc Receptors that Activate Mast Cells and Macrophage Via Tyrosine Kinases were Protected Against OA

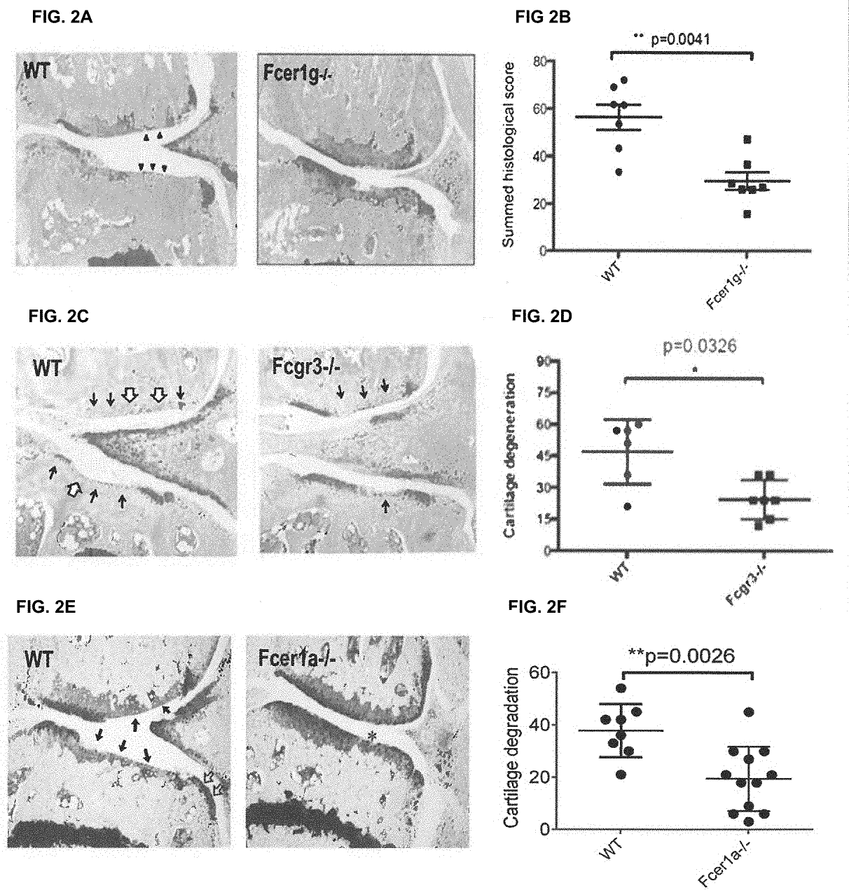

Mice deficient in FcR signaling or the activating FcRs, Fcgr3 and Fcer1a were induced by DMM to develop OA. 20 weeks following surgery, their knee joints were harvested, fixed and processed for histology. Tissue sections were stained by safranin-o to visualize cartilage damage and inflammation. FIGS. 2A-2F are representative knee joint sections and graphs illustrating reduction in cartilage damage 20-weeks following DMM surgery in mice lacking specific Fc receptors. FIG. 2A shows representative safranin-o stained knee joint sections from wild-type (WT) and Fc gamma common chain-deficient (Fcerlg−/−) mice. Major cartilage damage as evaluated by profound loss of proteoglycans or red staining is indicated by black arrowheads. FIG. 2B shows summed cartilage damage scores for the groups of WT (closed circles) and Fcerlg−/− (closed squares) mice. FIG. 2C shows representative safranin-o stained knee joint sections from wild-type (WT) and activating Fc gamma receptor 3-deficient (Fcgr3−/−) mice. Major cartilage damage as evaluated by profound loss of proteoglycans or red staining is indicated by white block arrowheads and moderate damage is indicated by black arrows. FIG. 2D shows summed cartilage damage scores for the groups of WT (closed circles) and Fcgr3−/−(closed squares) mice. FIG. 2E shows representative safranin-o stained knee joint sections from wild-type (WT) and high affinity IgE receptor Fc epsilon receptor 1 alpha-deficient (Fcerla−/−) mice. Major cartilage damage as evaluated by profound loss of proteoglycans or red staining is indicated by black arrows and moderate damage is indicated by asterisk. FIG. 2F shows summed cartilage damage scores for the groups of WT and Fcerla−/− mice. Statistical analyses were done by unpaired Student's ttest.

Thus, we demonstrated that genetic deficiency in FcR molecules that trigger or are associated with tyrosine kinase signaling pathways, reduced the severity of osteoarthritis in, and reduced joint inflammation in, mice induced to develop OA via DMM.

### Example 3

### Mice Deficient for Csf1, the Ligand for a Receptor Tyrosine Kinase, were Protected Against OA

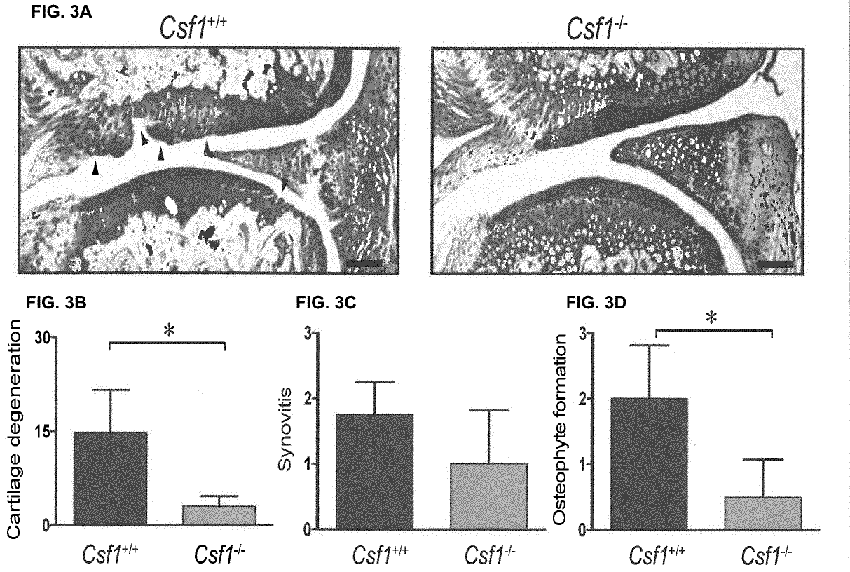

Mice deficient in Csf1 (Fms), whose signaling is transduced by its high affinity receptor, the CSF-1R, a receptor tyrosine kinase (RTK) and the cellular homologue of the v-fms oncoprotein, were induced to develop OA via DMM surgery. 20 weeks following surgery, their knee joints were harvested, fixed and processed for histology. Tissue sections were stained by safranin-o to visualize cartilage damage and inflammation. FIGS. 3A-3D show the results of experiments demonstrating that genetic elimination of MCSF and consequently macrophages significantly diminishes osteoarthritis-like pathologies in mice following destabilization of the medial meniscus. FIG. 3A shows representative toluidine blue stained joint-tissue sections from wild-type (Csf1^{+/+}) and Csf-deficient (Csf1^{-/-}) mice 20-weeks following destabilization of the medial meniscus (DMM) surgery. Arrowheads denote areas of cartilage damage. FIGS. 3B-3D are bar graphs showing histological scores of cartilage damage, synovitis and osteophyte formation in mice as described in FIG. 3A, respectively. *P<0.05 and by unpaired Student's ttest.

Thus, we demonstrated that genetic deficiency in Csf1 that activates macrophage via a receptor tyrosine kinase, reduced the severity of osteoarthritis of, and reduced joint inflammation in, mice induced to develop of DMM-induced OA.

### Example 4

### Mice Lacking Expression of the Receptor Tyrosine Kinase, Kit were Protected Against the Development of DMM-Induced OA

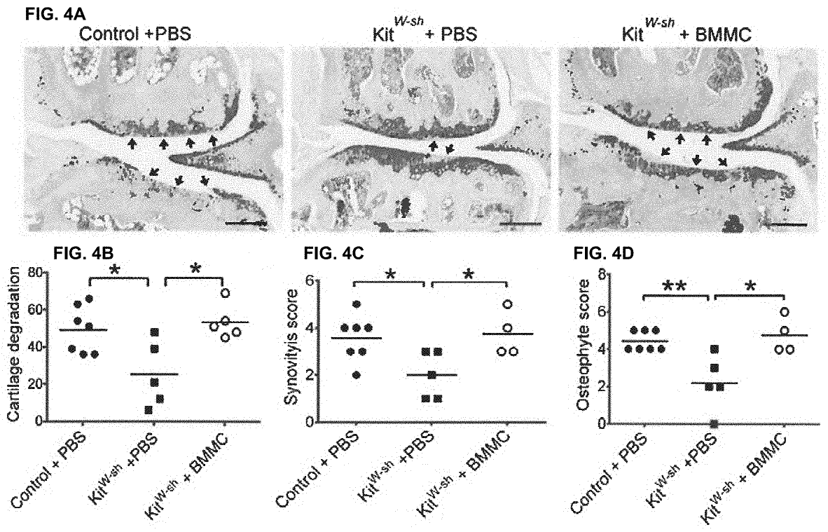

Wild-type mice, and mice deficient in Kit, a receptor tyrosine kinase (RTK) crucial for mast cell development, were induced to develop OA via DMM surgery. 4-week-old Kit-mutant mice were also reconstituted with mast cells by intravenous injection with 10⁷ wild-type bone marrow derived mast cells (BMMC) and 8 weeks later with 106 BMMCs via intraarticular injection. 20 weeks following surgery, their knee joints were harvested, fixed and processed for histology. Tissue sections were stained by safranin-o to visualize cartilage damage and inflammation. FIG. 4A shows representative knee joint sections stained with safranin-o from control mice (left panel), mast cell deficient (Kit^{W-sh}) mice (middle panel) that received PBS i.e., no mast cells and mast cell reconstituted (right panel) i.e., Kit^{W-sh} mice that received bone marrow-derived mast cells, 20-weeks after DMM surgery. Arrows indicate areas of cartilage damage. FIGS. 4B-4D are graphs showing histological scores of cartilage damage, synovitis and osteophyte formation in mice as described in FIG. 4C, respectively. *P<0.05 and **P<0.01 by unpaired Student's ttest.

Thus, we demonstrated that genetic deficiency in the receptor tyrosine kinase, crucial for mast cell development, reduced the severity of osteoarthritis in, and reduced joint inflammation in, mice induced to develop of DMM-induced OA.

### Example 5

### Systemic Treatment with the Tyrosine Kinase Inhibitor Imatinib Protected Mice Against the Development of OA

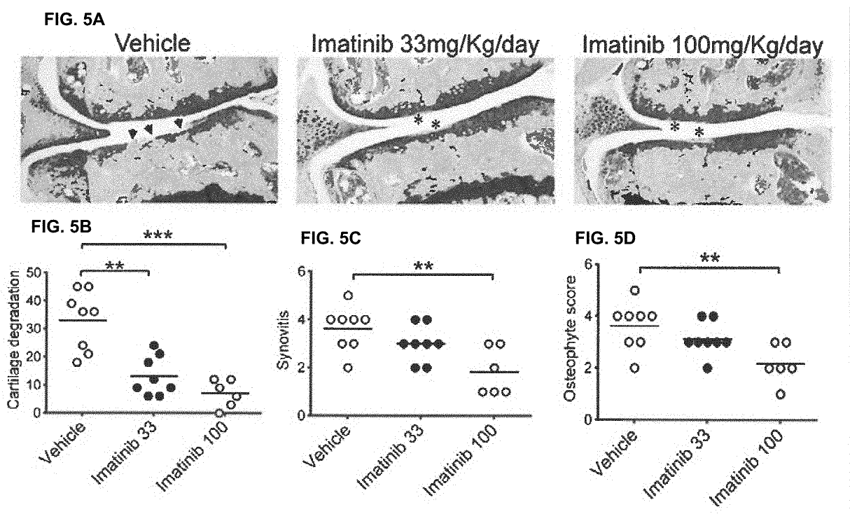

Wild-type mice were surgically-induced to develop OA by DMM. 24 hrs following surgery, mice were treated systemically with the tyrosine kinase inhibitor, imatinib, at doses of 33 mg/kg/day or 100 mg/kg, given orally twice-daily for 12 weeks starting one day after DMM surgery. FIG. 5A shows representative safranin-o stained knee joint sections from vehicle (left panel), imatinib 33 mg/Kg/day (middle panel), and imatinib 100 mg/Kg/day (right panel) treated mice. Arrows indicate areas of cartilage damage. FIG. 5B-5D are graphs showing histological scores of cartilage damage, synovitis and osteophyte formation in vehicle (circles), imatinib 33 mg/Kg/day (squares), and imatinib 100 mg/Kg/day (triangles) treated mice, respectively. Each symbol represents scores from individual mice and line represents the mean values for these scores. *P<0.05, **P<0.01 and ***P<0.001 by unpaired Student's ttest.

Thus, we demonstrated that systemic treatment with the tyrosine kinase inhibitor imatinib prevented the development of DMM-induced OA.

### Example 6

### Sustained Release Tyrosine Kinase Inhibitor Nanoparticles Prolonged the Residency of Tyrosine Kinase Inhibitors in Human Synovial Fluid

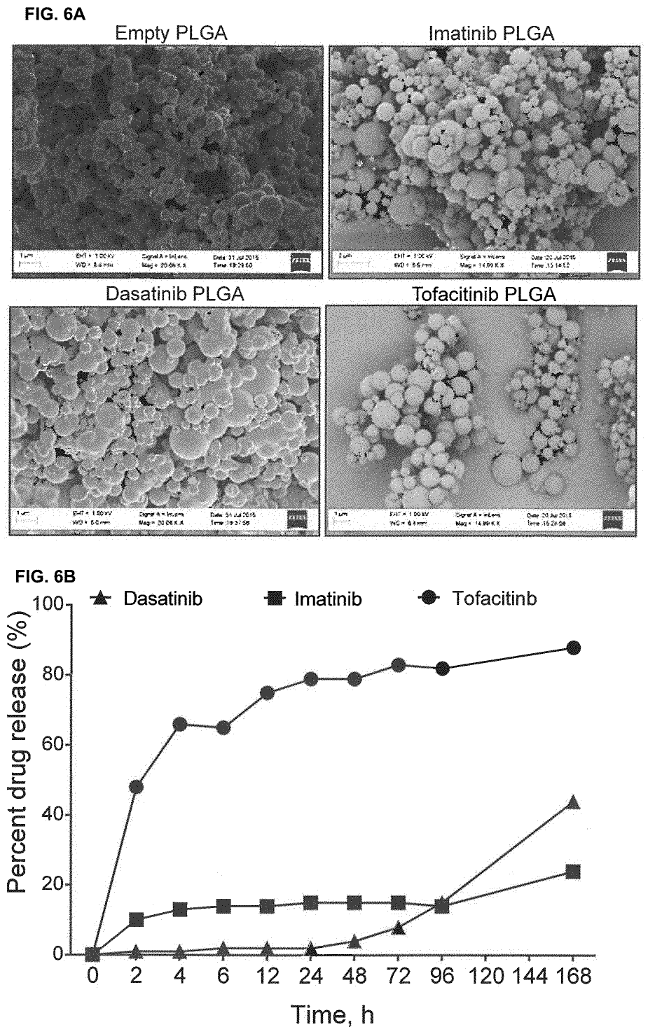

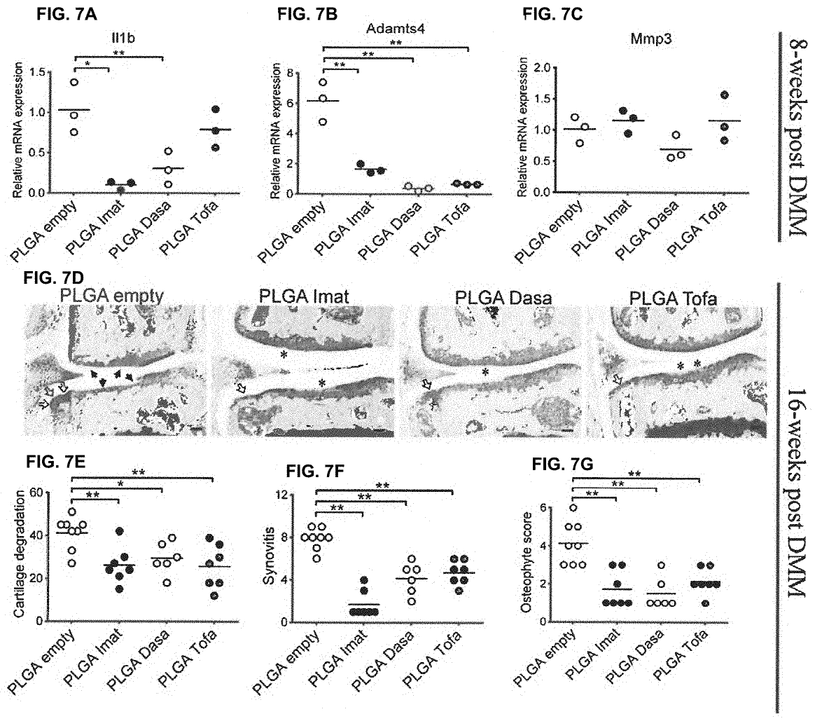

FIG. 6A shows representative scanning electron microscopy images of PLGA particles without any drug (empty PLGA) or with any of the 3 TKIs tested i.e., imatinib, tofacitinb or dasatinib. The PLGA formulations had an average particle size ranging from 20 nm-2 um. FIG. 7B is the result of experiments demonstrating the release of drugs from the PLGA encapsulations over time in 5% simulated synovial fluid containing hyaluronic acid as analyzed by mass spectrometry.

Thus, we demonstrated that PLGA-encapsulated tyrosine kinase inhibitor nanoparticles (TKI/PLGA nanoparticles) exhibited either a unimodal release (immediate also known as “burst” or sustained), or bimodal release (immediate also known as “burst” or prolonged and sustained) in human synovial fluid.

### Example 7

### Intraarticular Injection Treatment with Sustained Release Tyrosine Kinase Inhibitors (TKI Particles) Reduced Inflammation and Protected Mice Against DMM-Induced OA

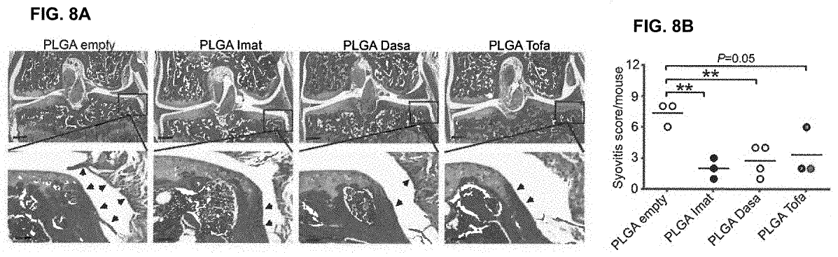

Wild-type mice were surgically-induced to develop OA by DMM. Mice were given intraarticular injections containing 50 μl of these different TKI/PLGA particle formulations every 3 weeks for 8 weeks or 16 weeks. Control mice received only PLGA particles denoted as PLGA empty in these graphs. FIG. 8A-8B are graphs showing relative mRNA expression of II1b and Adamts4, key pathogenic mediators of osteoarthritis in the synovium of mice described above. Symbols denote individual mice and line represent mean values. FIG. 8C is a graph showing no change in Mmp3 gene expression in the synovium of mice treated with PLGA/imat, PLGA/Dasa or PLGA/Tofa. Control mice received only PLGA particles denoted as PLGA/empty in these graphs. *P<0.05 and **P<0.01 by unpaired Student's t test. FIG. 8D shows representative knee joint sections stained with safranin-o from mice treated with vehicle (PLGA/empty), imatinib (PLGA/imat), dasatinib (PLGA/dasa) or tofacitinib (PLGA/tofa). Asterisk denotes areas of moderate cartilage damage, arrows indicate areas of severe cartilage damage. FIG. 8E-8G are graphs showing histological scores of cartilage damage, synovitis and osteophyte formation in mice described in FIG. 8D, respectively. *P<0.05, and **P<0.01 by unpaired Student's ttest.

Thus, we demonstrated that PLGA sustained release formulations of the tyrosine kinase inhibitors imatinib, dasatinib and tofacitinib (TKI/PLGA particles) reduced inflammation and protected against the development of DMM-induced OA.

### Example 8

### Intraarticular Injection Treatment with Sustained Release Tyrosine Kinase Inhibitors (TKI Particles) Reduced the Severity of RA in the CAIA Mouse Model

Mice were induced to develop RA using the collagen antibody-induced arthritis (CAIA) model. To induce CAIA, all mice were administered given 1 mg of Arthrogen-CIA® 5-Clone monoclonal antibody cocktail i.p. on day 0, and 25 μg LPS i.p. on day 3. On day 4, CAIA-challenged mice were given a single intraarticular injection of PLGA/empty, PLGA/Imat, PLGA/dasa or PLGA/tofa. On day 11 mice were sacrificed, their knee joints harvested and processed for histology. FIG. 8A show representative H&E stained knee joint sections from CAIA-challenged mice that received no treatment (PLGA/empty), imatinib (PLGA/Imat), dasatinib (PLGA/dasa) or tofacitinib (PLGA/tofa). Bottom panels are magnified photomicrographs denoting synovial inflammation (arrows) in each of these cases. FIG. 8B show the summed synovitis score from knee joint sections of mice described in FIG. 8A. Symbols denote individual mice and bars denote mean values. *P<0.05, **P<0.01 by unpaired Student's t test.

Thus, we demonstrated that PLGA sustained release formulations of the tyrosine kinase inhibitors imatinib, dasatinib and tofacitinib reduced the severity of RA in the CAIA mouse model.

### Example 9

### Intraarticular Injection of Sustained Release Tyrosine Kinase Inhibitor Treatment Reduced the Severity of Crystal-Induced Arthritis in Mice

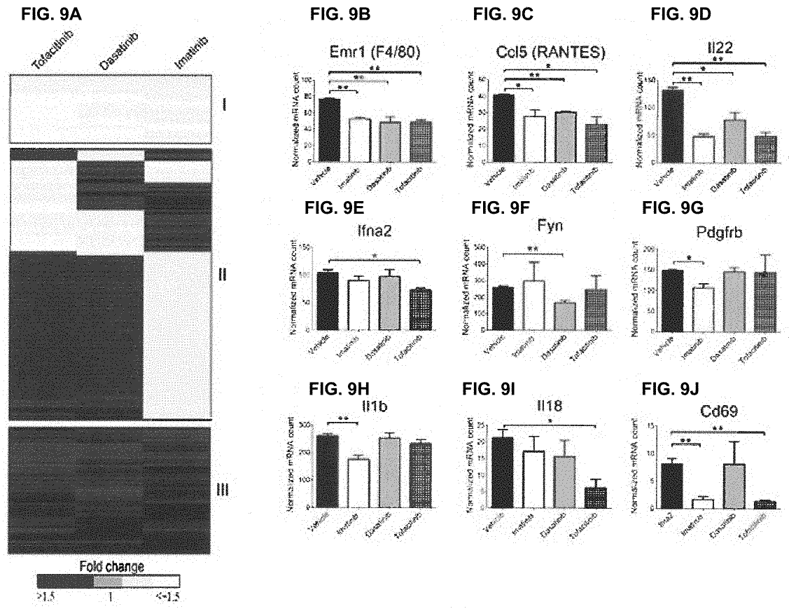

Mice were induced to develop crystal-induced arthritis using monosodium urate (MSU, 5 mg/ml) crystal-induced model of gouty arthritis. Mice were given a single intraarticular injection of individual TKI/PLGA formulation at 4 h after MSU crystal injection in the knees. FIG. 9A is a Nanostring-based heatmap depicting fold changes of over 300 genes in the local knee joint of mice obtained at 24 hrs after gouty arthritis induction. Fold changes of individual TKI/PLGA treated mice were those over vehicle (PLGA/empty) treated mice. I— set of genes whose expression was significantly lower in all three treatment groups compared to vehicle. II— set of genes whose expression was significantly lower in at least one drug treatment group compared to vehicle. III— set of genes whose expression remained unaltered in all three treatment groups relative to vehicle. FIG. 9B-9J shows bar graphs representing examples of genes whose local expression has been lowered following treatment with TKI/PLGA formulation. *P<0.05 and **P<0.01 by unpaired Student's ttest.

Thus, we demonstrated that PLGA sustained release formulations of the tyrosine kinase inhibitors imatinib, dasatinib and tofacitinib reduced the severity of crystal-induced mouse arthritis.

### Example 10

### Systemic Sustained Release Tyrosine Kinase Inhibitor Treatment (with TKI/PLGA Particles) Reduces the Severity of OA in Mice

Mice (n=10 per experimental arm) will be induced by DMM surgery to develop OA. On day 1 following surgery mice will be treated with PLGA/imat, PLGA/Dasa or PLGA/Tofa by oral gavage once a week for 12 weeks. Control mice will receive only PLGA particles (PLGA/empty) following the same regimen as for the treatment mice. 12 weeks after surgery, mice will be sacrificed and their knee joints harvested for histological examination of cartilage damage, synovitis, mast cell numbers and inflammation. Systemic administration of PLGA/TKI reduces cartilage damage, osteophyte formation and synovial inflammation in mice. Analyses of mast cell numbers shows lower mast cell counts as well as decreased numbers of mast cells that have degranulated in the synovium of PLGA/TKI-treated mice as compared to controls.

Thus, systemic sustained release tyrosine kinase inhibitors (TKI/PLGA particles) reduces the severity and progression of OA in mice.

### Example 11

### Systemic Sustained Release Tyrosine Kinase Inhibitor Treatment (with TKI/PLGA Particles) Reduces the Severity of Crystal-Induced Arthritis in Mice

Mice (n=5 per experimental arm) will be induced to develop crystal-induced arthritis using monosodium urate (MSU, 5 mg/ml). 4 hrs after induction, mice will be treated orally with PLGA/imat, PLGA/Dasa or PLGA/Tofa. Control mice will receive only PLGA particles (PLGA/empty). 24 hrs after induction mice are sacrificed and their knee joints harvested for histologic analysis and RNA extraction and subsequent qPCR analyses for changes in inflammatory gene expression. Local inflammatory gene expression is significantly lower in mice that receive PLGA/TKI formulations as compared to controls. Histologic analysis, including hematoxylin and eosin staining of joint sections, reveals statistically lower numbers of inflammatory cells in mice that receive PLGA/TKI formulations as compared to controls.

Thus, systemic sustained release tyrosine kinase inhibitors (TKI/PLGA particles) reduce inflammation associated with crystal-induced arthritis in mice.

### Example 12

### Intra-Nasal Administration of Sustained Release Tyrosine Kinase Inhibitor (TKI/PLGA) Formulations for the Treatment of Allergic Asthma in Mice

Mice (n=10 per experimental arm) are induced to develop asthma using the house dust mite (HDM)-induced asthma model. Mice are inoculated intranasally on day 0, 1 and 2 with 25 μg HDM (sensitization phase) and on day 14, 15, 18 and 19 with 6.25 μg HDM (challenge phase). Inoculum volume is 20 μl for every HDM and saline exposure and inoculation procedures are performed during isoflurane inhalation anesthesia. On day 14, mice are initiated on once per week, or alternatively twice per week, intranasal administration of PLGA/TKI formulations i.e., PLGA/lmat or PLGA/Dasa or PLGA/Tofa. Controls receive isotonic sterile saline intranasally on each occasion and receive PLGA/empty formulations on day 14. The experiment is terminated at day 42 by euthanizing the mice and the subsequent collection and processing of samples: in one experiment bronchoalveolar lavage fluid (BALF) and citrated blood is collected, in a separate experiment one lung is obtained for pathology/histology and one lung for homogenization to extract proteins or RNA. Analysis of BALF reveals significant diminution in inflammatory cells, serum cytokine levels are significantly lower in mice receiving either once per week or twice per week intranasal administration of PLGA/TKI. Airway hyper-responsiveness (AHR) is also significantly lower in PLGA/TKI-treated groups relative to controls. Histological assessment shows significant reduction in mucus containing goblet cells and inflammatory cell infiltrate including fewer mast cells in PLGA/TKI-treated groups relative to controls.

Thus, intranasal administration of sustained-release TKI/PLGA particles reduces the severity of allergic asthma in mice.

### Example 13

### Inhalation of Sustained Release Tyrosine Kinase Inhibitor (TKI/PLGA) Formulations for the Treatment of Allergic Asthma in Mice

Mice (n=10 per experimental arm) are induced to develop asthma using the house dust mite (HDM)-induced asthma model. Mice are inoculated intranasally on day 0, 1 and 2 with 25 μg HDM (sensitization phase) and on day 14, 15, 18 and 19 with 6.25 μg HDM (challenge phase). Inoculum volume is 20 μl for every HDM and saline exposure and inoculation procedures are performed during isoflurane inhalation anesthesia. On day 14, mice are initiated on every 3 day, or alternatively every 7 day, aerosol inhalation various PLGA/TKI formulations i.e., PLGA/Imat or PLGA/Dasa or PLGA/Tofa. Controls receive isotonic sterile saline intranasally on each occasion and receive aerosol inhalation PLGA/empty formulations every 3^{rd} or every 7^{th} day. The experiment is terminated at day 42 by euthanizing the mice and the subsequent collection and processing of samples: in one experiment bronchoalveolar lavage fluid (BALF) and citrated blood is collected, in a separate experiment one lung is obtained for pathology/histology and one lung for homogenization to extract proteins or RNA. Analysis of BALF reveals significant diminution in inflammatory cells, serum cytokine levels (e.g., IL13, TNFalpha [TNFa]) are significantly lower in mice receiving every third day, or every seventh day, PLGA/TKI. AHR is also significantly lower in PLGA/TKI-treated groups relative to controls. Histological assessment shows significant reduction in mucus containing goblet cells and inflammatory cell infiltrate including fewer mast cells in PLGA/TKI-treated groups relative to controls.

Thus, inhaled administration of sustained-release TKI/PLGA particles reduces the severity of allergic asthma in mice.

### Example 14

### Intra-Nasal Administration of Sustained Release Tyrosine Kinase Inhibitor (TKI/PLGA) Formulations for the Treatment of Allergic Rhinitis in Mice

Mice (n=10 per experimental arm) are immunized with ovalbumin (10 μg OVA) emulsified in 2 mg AL(OH)3 (OVA/alum) in 0.5 ml PBS or as a control with PBS in alum by an intraperitoneal injection on day 0 and day 7. Ten days later, mice are challenged by instilling a droplet of 10 μl OVA (1 μg/ml) in each nostril with a micropipettor on three successive days a week for three consecutive weeks. Treatment with PLGA/TKI i.e., PLGA/Imat or PLGA/Dasa or PLGA/Tofa or PLGA/empty (vehicle control) is given together with the OVA challenge via intranasal administration. The control group is sensitized to OVA but is given a challenge with PBS in the presence of diluent (PBS-dil). Twenty-four hours after the last OVA or PBS application, mice are sacrificed using anesthetic overdose followed by bleeding. The palatine containing the nasal mucosa is snap frozen in freezing solution. Before and after sensitization, the frequencies of nasal symptoms (sneezing, nasal rubbing) are recorded and the serum levels of total immunoglobulin E (IgE) are evaluated using ELISA. Finally, the murine nasal mucosal tissues are snap frozen and stained by Giemsa solution to estimate the degree of mast cell infiltration. Mice that receive PLGA/TKI formulations show significant reduction of nasal symptoms (sneezing and rubbing) relative to those that receive PLGA/empty (vehicle control). Total IgE levels are also lower in PLGA/TKI-treated mice compared to vehicle controls. Finally, mast cell infiltration and degranulation is also found to be statistically decreased in PLGA/TKI-treated groups relative to vehicle control groups.

Thus, intra-nasal administration of sustained-release TKI/PLGA particles reduces the severity of allergic rhinitis in mice.

### Example 15

### Inhalation of Sustained Release Tyrosine Kinase Inhibitor (TKI/PLGA) Formulations for the Treatment of Allergic Rhinitis in Mice

Mice (n=10 per experimental arm) are immunized with ovalbumin (10 μg OVA) emulsified in 2 mg AL(OH)3 (OVA/alum) in 0.5 ml PBS or as a control with PBS in alum by an intraperitoneal injection on day 0 and day 7. Ten days later, mice are challenged by instilling a droplet of 10 μl OVA (1 μg/ml) in each nostril with a micropipettor on three successive days a week for three consecutive weeks. On Day 17, treatment with PLGA/TKI i.e., PLGA/Imat or PLGA/Dasa or PLGA/Tofa or PLGA/empty (vehicle control) is initiated via aerosol inhalation, and is administered every three days or every seven days. The control group is sensitized to OVA but is given a challenge with PBS in the presence of diluent (PBS-diluent). Twenty-four hours after the last OVA or PBS application, mice are sacrificed using anesthetic overdose followed by bleeding. The palatine containing the nasal mucosa is snap frozen in freezing solution. Before and after sensitization, the frequencies of nasal symptoms (sneezing, nasal rubbing) are recorded and the serum levels of total immunoglobulin E (IgE) are evaluated using ELISA. Finally, the murine nasal mucosal tissues are snap frozen and stained by Giemsa solution to estimate the degree of mast cell infiltration. Mice that inhale PLGA/TKI formulations show significant reduction of nasal symptoms (sneezing and rubbing) relative to those that receive PLGA/empty (vehicle control). Total IgE levels are measured and are statistically lower in PLGA/TKI-treated mice compared to vehicle controls. Finally, mast cell infiltration and degranulation is found to be significantly decreased in PLGA/TKI-treated groups relative to vehicle control groups.

Thus, inhaled administration of sustained-release TKI/PLGA particles reduces the severity of allergic rhinitis in mice.

### Example 16

### Intra-Nasal Administration of Sustained Release Tyrosine Kinase Inhibitor (TKI/PLGA) Formulations for the Treatment of Nasal Polyps in Mice

Mice (n=10 per experimental arm) are induced to develop OVA-induced allergic rhinitis as in example 14 and example 15. After induction of an ovalbumin (OVA)-induced allergic rhinosinusitis, 6% OVA and staphylococcal enterotoxin B (SEB) (10 ng) are instilled into the nasal cavity of mice 3 times a week for 8 weeks. Beginning at week 3 of challenge with OVA and SEB, mice will receive intra-nasal administration of PLGA/TKI formulations i.e., PLGA/Imat or PLGA/Dasa or PLGA/Tofa or PLGA/empty (vehicle control) once every 3 or once every 7 days. The murine nasal mucosal tissues are snap frozen and stained by Giemsa solution to estimate the degree of mast cell infiltration and by H&E to assess inflammation. Mice that receive PLGA/TKI formulations develop small polyps and lesser inflammation relative to vehicle controls. Mast cell infiltration and degranulation is lower in the PLGA/TKI group relative PLGA/Empty group.

Thus, intra-nasal administration of sustained-release TKI/PLGA particles reduces the severity of nasal polyps in mice.

Mouse model of nasal polyposis. BALB/c mice (four weeks of age) are randomized into one control (group A; n=10) and three experimental groups (each of n=10). Eosinophilic inflammation of the sinonasal (e.g. sinus and nasal) mucosa is induced, and Staphylococus aureus enterotoxin B contributes to induction of nasal polypoid lesions in an allergic rhinosinusitis mouse model (Chang, D Y et al. (2015). Therapeutic effects of intranasal cyclosporine for eosinophilic rhinosinusitis with nasal polyps in a mouse model. American journal of rhinology & allergy, 29(1), e29-e32 doi: 10.2500/ajra.2015.29.4152). In group A, phosphate buffered saline (PBS) is instilled intranasally. The experimental groups are as follows: intranasal instillation of polymer particle (group B); TKI/polymer particle (group C); triamcinolone acetonide (TAC) (group D). Mice in the experimental groups are systemically sensitized with 25 g of ovalbumin (OVA) (grade V; Sigma, St. Louis, Mo.) dissolved in 300 L of PBS, in the presence of 2 mg of aluminum hydroxide gel as an adjuvant, by i.p. injection on days zero and five. One week after the second i.p. injection, mice in the control and experimental groups are challenged intranasally with PBS and 3% OVA diluted in 40 L of PBS, respectively, daily for one week. Thereafter, continual intranasal challenge is maintained in the same manner three times per week for four consecutive weeks. Finally, 3% OVA diluted in 40 L of PBS is applied intranasally accompanied by the administration of drugs, including TKI/polymer particle and TAC, at the same intervals for eight consecutive weeks. During that period, 10 ng of Staphylococcus aureus enterotoxin B diluted in 20 L of PBS is challenged intranasally subsequent to the instillation of OVA once weekly. Twenty-four hours after the final nasal challenge with drug administration, mice are euthanized and decapitated. Four control mice and six from each experimental group are prepared for histologic examination; the sinonasal mucosa from four mice in each group are used for quantitative measurement of cytokines using a cytometric bead array. This study is approved by the Stanford University Committee of Animal Research and in accordance with NIH guidelines.

Histologic Analyses The heads of the mice are fixed immediately in 2% paraformaldehyde and decalcified in 5% nitric acid for four to five days at 4° C. The tissues are dehydrated and processed according to standard paraffin-embedding procedures. The true maxillary sinus and ethmoidal labyrinths are identified at the lesions posterior to the two maxillary turbinelles. Two coronal sections that are similar to the sinus cavity were chosen for evaluation. Hematoxylin and eosin, Sirius red, and Toludine blue staining are used to examine polyp formation, eosinophilic inflammation, and mast cells, respectively. The numbers of polyp-like lesions, eosinophils, and mast cells are counted in high-power fields (original magnification, 400). Two consecutive slides are reviewed to obviate processing errors. At killing, sinonasal mucosae are dissected and harvested, the obtained mucosae are homogenized mechanically, and multiplex cytokine analysis performed.

At killing, sinonasal mucosae are dissected and harvested. The obtained mucosae are homogenized mechanically and resuspended in PBS. The homogenates are filtered and filtrates are then centrifuged. After centrifugation, supernatants are collected and cryopreserved at 70° C. until the time of analysis. Concentrations of murine cytokines including tumor necrosis factor (TNF)/interferon (IFN)-/interleukin (IL)-5/IL-13, and IL-2/IL-4/IL-17A, IL-16, IL-6, are assessed by cytometric bead arrays.

Polyp-like lesions are present only at the junction of olfactory and respiratory epithelia. No polyp-like lesions were observed in group A. Eleven lesions are observed in six mice in group B. Three and seven lesions are observed in groups C and D, respectively. At the junction of the olfactory and respiratory epithelia, the number of eosinophils and mast cells was highest in group B and decreased significantly in groups C (TKI/polymer particle group). There is no definite infiltration of inflammatory cells in group A. Quantitative measurement of cytokine levels including TNF, IL-2, IFN-, IL-4, IL-5, IL-6, IL-13, IL-16, and IL-17A, are markedly elevated in group B compared with group A. TNF, IL-4, IL-5, IL-6, IL-13, IL-16, and IL-17A levels are significantly reduced in groups C.

Thus, intra-nasal administration of sustained-release TKI/PLGA particles reduces the severity of nasal polyps in mice.

### Example 17

### Inhaled Administration of Sustained Release Tyrosine Kinase Inhibitor (TKI/PLGA) Formulations for the Treatment of Nasal Polyps in Mice

Mice (n=10 per experimental arm) are induced to develop OVA-induced allergic rhinitis as in example 14 and example 15. After induction of an ovalbumin (OVA)-induced allergic rhinosinusitis, 6% OVA and staphylococcal enterotoxin B (SEB) (10 ng) are instilled into the nasal cavity of mice 3 times a week for 8 weeks. Beginning at week 3 post-challenge with OVA and SEB, mice are treated with PLGA/TKI formulations i.e., PLGA/Imat or PLGA/Dasa or PLGA/Tofa or PLGA/empty (vehicle control) once every 3 or once every 7 days via aerosol inhalation. The murine nasal mucosal tissues are snap frozen and stained by Giemsa solution to estimate the degree of mast cell infiltration and by H&E to assess inflammation. Mice that receive PLGA/TKI formulations develop statistically smaller polyps and lesser inflammation relative to vehicle controls. Mast cell infiltration and degranulation is lower in the PLGA/TKI group relative PLGA/Empty group.

Thus, inhaled administration of sustained-release TKI/PLGA particles reduces the severity of nasal polyps in mice.

### Example 18

### Inhaled Administration of Sustained Release Tyrosine Kinase Inhibitor (TKI/PLGA) Formulations for the Treatment of Aspirin-Exacerbated Respiratory Disease (AERD) in Mice

Mice (n=10 per experimental arm) are induced to develop AERD by house dust mite priming of mice lacking microsomal PGE2 synthase (ptges(−/−)) as described (Liu, T et al. (2015). Aspirin-Exacerbated Respiratory Disease Involves a Cysteinyl Leukotriene-Driven IL-33-Mediated Mast Cell Activation Pathway. The Journal of Immunology, 195(8), 3537-3545 doi: 10.4049/jimmunol.1500905 Liu, T et al. (2013). Prostaglandin E2 deficiency causes a phenotype of aspirin sensitivity that depends on platelets and cysteinyl leukotrienes. Proceedings of the National Academy of Sciences, 110(42), 16987-16992 doi: 10.1073/pnas.1313185110). These mice exhibit similar histologic and molecular features to those observed in humans with AERD. Two weeks following priming with house dust mite, mice are treated with PLGA/TKI formulations i.e., PLGA/Imat or PLGA/Dasa or PLGA/Tofa or PLGA/empty (vehicle control) once every 3 or once every 7 days via aerosol inhalation. The experiment is terminated at day 48 by euthanizing the mice and the subsequent collection and processing of samples: in one experiment bronchoalveolar lavage fluid (BALF) and citrated blood is collected, in a separate experiment one lung is obtained for pathology/histology and one lung for homogenization to extract proteins or RNA. Analysis of BALF reveals significant diminution in inflammatory cells by flow cytometry, serum cytokine levels (e.g., IL13, IL33, and other pro-inflammatory cytokines) as measured by ELISA are significantly lower in mice receiving every third day, or every seventh day, PLGA/TKI. Histological assessment shows significant reduction in mucus containing goblet cells and inflammatory cell infiltrate including fewer mast cells and fewer eosinophils in PLGA/TKI-treated groups relative to controls.

Thus, inhaled administration of sustained-release TKI/PLGA particles reduces the severity of AERD in mice.

### Example 19. Inhaled Administration of Sustained Release Tyrosine Kinase Inhibitor (TKI/PLGA) Formulations for the Treatment of COPD in Mice

Mice (n=10 per experimental arm) are induced to develop COPD by cigarette smoke exposure as described (Vlahos, R et al. (2015). (In Press) Preclinical murine models of chronic obstructive pulmonary disease. European Journal of Pharmacology, 1-7 doi: 10.1016/j.ejphar.2015.03.029; Fricker, M et al. (2014). Animal models of chronic obstructive pulmonary disease. Expert opinion on drug discovery, 9(6), 629-645 doi: 10.1517/17460441.2014.909805.). These mice exhibit similar histologic and molecular features to those observed in humans with COPD starting at 3 months following initiation of cigarette smoke exposure. Three months following initiation of cigarette smoke exposure, mice are treated with PLGA/TKI formulations i.e., PLGA/Imat or PLGA/Dasa or PLGA/Tofa or PLGA/empty (vehicle control) once every 3 or once every 7 days via aerosol inhalation. The experiment is terminated at 6 months by euthanizing the mice and the subsequent collection and processing of samples: in one experiment bronchoalveolar lavage fluid (BALF) and citrated blood is collected, in a separate experiment one lung is obtained for pathology/histology and one lung for homogenization to extract proteins or RNA. Analysis of BALF reveals significant diminution in inflammatory cells by flow cytometry, serum cytokine levels as measured by ELISA are significantly lower in mice receiving every third day, or every seventh day, PLGA/TKI. Histological assessment shows statistically significant reductions in inflammatory cell infiltrates including fewer mast cells in PLGA/TKI-treated groups relative to controls.

Thus, inhaled administration of sustained-release TKI/PLGA particles reduce the severity of COPD in mice.

### Example 20

### Example of Humans at High Risk for Development, or with Preclinical OA, or with Established OA and their Treatment with TKI/Polymer Particles

(1) A 59 year old male with knee pain is diagnosed with osteoarthritis of the R knee (Kellgren-Lawrence, K-L, grade II). He is limited when running and sitting for prolonged periods by the sensation of stiffness or “gelling” in his knee. His R knee range of motion is intact and there is no deformity of angulation of adduction moment on ambulation. Assessment is performed using the Western Ontario and McMaster Universities (WOMAC) OA index for assessment of pain, function and stiffness of the knee joint as well as a score of 1-100 using a visual analog score (VAS) for pain. The patient undergoes MRI with gadolinium of the R knee which reveals enhancement consistent with synovitis which is assessed using a semi-quantitative scoring system. The patient is treated with a low, medium, or high dose of Imatinib/PLGA delivered by intra-articular injection with follow up evaluation.

(2) 54 year old male presents with mild intermittent locking in his left knee. X-ray reveals K-L grade 1 OA and ultrasound demonstrates a degenerative meniscal tear and moderate synovial enhancement consistent with synovitis. The patient is offered arthroscopic meniscal debridement but declines surgical intervention. The patient is treated with a low, medium, or high dose of Imatinib/PLGA intra-articular injection with follow up evaluation for OA symptoms.

(3) 28 year old male develops a fracture of his right ankle (tibial plafond) with appropriate reduction and casting. His X-rays do not show any features of OA. Given the 30% risk of significant radiographic OA within 2-4 years increasing to 74 percent by 11 years after fracture, the patient is monitored for evidence of joint inflammation by ultrasound and MRI, and/or by molecular markers. Ultrasound detects a synovial effusion and synovitis, and as a result the patient is treated with a low, medium, or high dose of Imatinib/PLGA intra-articular injection with follow up evaluation for OA symptoms.

(4) 49 year old male presents with intermittent pain in his left knee. X-ray reveals K-L grade 1 OA and MRI demonstrates a degenerative meniscal tear and moderate synovial enhancement consistent with synovitis. The patient is offered arthroscopic meniscal debridement but declines surgical intervention. The patient is treated with a low, medium, or high dose of Imatinib/PLGA intra-articular injection with follow up evaluation for OA symptoms.

### Follow Up Evaluation:

Humans at risk for OA, with early OA, or with established OA are evaluated before and after treatment for the presence of inflammation in the involved joint to 1. identify individuals most-likely to respond to treatment with TKI/polymer and 2. evaluate response to TKI/polymer treatment. Testing for joint inflammation can be performed with imaging markers, such as MRI with or without gadolinium contrast, or an ultrasound, to determine if one or more of the following features indicative of inflammation are present: synovial enhancement or proliferation, an effusion is present, and bone marrow edema. Molecular markers of inflammation can also be tested for, including one or more of CRP, ESR and inflammatory cytokines. If an effusion is present, a joint aspiration can be done and the fluid analyzed for specific inflammatory markers. Finally, clinical history and exam can be used to assess inflammation—including the presence of an effusion on physical exam or morning stiffness on history. Various efficacy outcomes will be measured to evaluate effect of TKI/polymer therapy in pain reduction global assessment of disease, treatment, slowing, halting, prevention of OA. Assessment will include but not be limited to: 1) radiographic evaluation to assess inflammation and structural changes using baseline and post-treatment K-L scores 2) change from baseline in the pain the patient felt in the index knee while walking on a flat surface and in the patient's global assessment of response to therapy, averaged over weeks 1 through 16. 3) Change from baseline in overall knee pain and in scores on the WOMAC subscales for pain, stiffness, and physical function. Pain while walking and overall knee pain are recorded daily in an electronic diary, whereas the patient's global assessment of response to therapy and scores on the WOMAC subscales are recorded on study-visit days. Pain, the patient's global assessment, and scores on the WOMAC subscales are assessed with the use of a visual-analogue scale that range from 0 to 100. In the case of pain and WOMAC scores, a lower score indicate improvement (i.e., less pain, less stiffness, and less limitation of physical function), whereas in the case of the patient's global assessment, a higher score indicated improvement (i.e., a better response to therapy). 4) The response to therapy on the basis of the criteria of the Outcome Measures for Rheumatology Committee and Osteoarthritis Research Society International Standing Committee for Clinical Trials Response Criteria Initiative (OMERACT-OARSI). Patients are classified as having had a response if the WOMAC pain or physical-function score decreased by 50% or more and by 20 or more points on the visual-analogue scale or if two of the following three findings are recorded: a decrease in the WOMAC pain score by 20% or more and by 10 or more points on the visual-analogue scale, a decrease in the WOMAC physical-function score by 20% or more and by 10 or more points on the scale, or an increase in the score on the patient's global assessment by 20% or more and by 10 or more points on the scale.

### Example 21

### Intraarticular Sustained Release TKI Particles are Well Tolerated, Prolongs the Residency of Given TKI in the Synovial Joints, Reduces Inflammation, Tissue Damage and Pain in Patients with Knee Osteoarthritis

Intra-articular (IA) administration of corticosteroids for example, triamcinolone acetonide injectable suspension (TCA IR) are commonly used to treat pain and inflammation associated with osteoarthritis (OA) of the knee. While corticosteroids may relieve pain caused by osteoarthritis for a short amount of time (weeks to months), they are not effective in treating pathological inflammatory processes in OA. Tyrosine kinase inhibitors are capable of inhibiting several inflammatory pathways of pathogenic cell types like mast cells. Imatinib/PLGA particles used in this study are an extended-release IA formulation of Imatinib at a load dose of about 10% in 50:50 poly(lactic-co-glycolic acid) (PLGA) nanoparticles that is intended to deliver said TKI to the synovial and peri-synovial tissues for a period of up to 3 months.

This study is designed as a double-blind, randomized, placebo-controlled, four parallel arm, dose-finding study, to demonstrate the efficacy of single intraarticular (IA) injections of TKI/PLGA particle formulations in patients with symptomatic osteoarthritis (OA) of the knee. Approximately 80 male and female patients 40-80 years old, with BMI <30 kg/m² and with a clinical diagnosis of symptomatic primary osteoarthritis of the knee will be randomized to a total of 4 treatment arms. Each arm includes a single intraarticular injection of one of three dosages of TKI/PLGA particle formulations (low, intermediate and high dose) OR placebo. The randomization ratio will be 1:1:1:1. The validated Western Ontario and McMaster University questionnaire (WOMAC) will be used to measure total knee pain choosing its visual analogue scale version (VAS). The WOMAC VA 3.1 A subscore (WOMAC A) ranges from 0 to 500 mm (summing up five VAS 0-100 mm) with higher scores indicating more pain. The primary efficacy variable will be the change of the Western Ontario and McMaster Universities Visual Analogue Scale 3.1 A (WOMAC VA 3.1 A) (total pain) subscore from baseline up to 24 weeks after randomization. The secondary outcome measures include (a) Change in WOMAC INDEX: The WOMAC VA 3.1 Index score (WOMAC INDEX) is the sum of WOMAC A (total pain), WOMAC B (stiffness) and WOMAC C (functional impairment) subscores. The WOMAC INDEX score ranges from 0 to 2400 mm, with higher scores indicating higher disease burden. (b) Responder Rate According to OMERACT-OARSI Criteria: Percentage of responders according to Outcome Measures in Rheumatology-Osteoarthritis Research Society International criteria (OMERACT-OARSI criteria). Patients with at least 50% improvement in pain or in function scores are considered responders. Alternatively, patients are considered responders if they show at least 20% improvement in at least two of the following scores: pain, function and Patients' Global Assessment (PGA) scores. Safety will be assessed by monitoring adverse events (AE) and clinical laboratory tests; local tolerability at the injection site will also be assessed. Other secondary outcomes include patient's global assessment, function of the target joints, ESR and serologic markers of inflammation (blood tests), duration of morning stiffness, number of tender and swollen joints, number of analgesic pills, cumulative dose of glucocorticoids, NSAIDs or colchicine and safety. Patient's global assessment of their general health is evaluated using a Lickert scale ranging from 0 to 10. Functional impairments are determined by asking the patient to assess function in the involved joints (3=total disability, 2=movement possible, 1=weight bearing possible, 0=painless full function. This study is designed to evaluate, by magnetic resonance imaging or MRI, knee cartilage and structure in all subjects. Clinical examinations and MRI are performed at baseline, and after 6, 12 and 24 weeks. Cartilage volume, thickness and surface area are determined in cartilage plates and subregions were defined using proprietary software. In addition, the population pharmacokinetics and the exposure-response relationship will be evaluated.

All treatments are well tolerated. The TKI/PLGA particle formulations maintain a gradient between synovial and systemic concentrations for the duration of this 24-week study. TK/PLGA particle formulations provide clinically meaningful and statistically significant improvement in the primary end point measure over placebo administration. The percentage of patients with an improvement in pain relief over the baseline level of pain, as measured at week 6 is statistically larger for the TKI/PLGA particle formulations as compared to the placebo. The percentage of patients with an improvement in pain relief and function over the baseline level of pain and function, as measured at week 4, week 8, week 12, week 16, week 20 and week 24 is statistically larger for the TKI/PLGA particle formulations as compared to placebo. The percentage of patients showing radiographic improvement in cartilage characteristics measured using pre-specified parameters over the baseline level of cartilage damage, as measured at week 6, week 12 and week 24 is larger for the TKI/PLGA particle formulations as compared to placebo.

Thus, we demonstrate that the TKI/PLGA particle formulations have the potential to provide prolonged suppression of the synovitis of OA, slow down or arrest tissue damage, effects that may prove beneficial to patients in the extension of symptomatic relief.

### Example 22

### Intraarticular Sustained Release TKI Particles are Well Tolerated and Reduce Inflammation, Tissue Damage and Pain Associated with Gouty Arthritis

This study is designed as a double-blind, randomized, placebo-controlled, four parallel arm, dose-finding study, to evaluate the efficacy of single intraarticular (IA) injections of TKI/PLGA particle formulations in patients with acute gouty arthritis in a specific joint. Approximately 80 male or female patients 20-80 years old, who meet at least 6 of the 12 American College of Rheumatology preliminary criteria (1977) for the classification of acute arthritis of primary gout, and have current or prior tophus or documented monosodium urate (MSU) crystals in the joint fluid, have serum uric acid >7.5 mg/dL. Each arm includes a single intraarticular injection of TKI/PLGA particle formulations (low, intermediate and high dose) OR placebo. The randomization ratio will be 1:1:1:1. The primary efficacy variable is improvement in pain relief, erythema, tenderness, swelling and inflammation in the joint with acute gouty arthritis from baseline as compared to the following time points: 1 day, 3 days, 5 days, 7 days, 10 days, 14 days, 21 days, and 28 days post-TKI/PLGA particle treatment. The reductions in joint pain, erythema, swelling and inflammation are consistently larger for the TKI/PLGA particle treated joints as compared to placebo treated joints.

Thus, we demonstrate that the TKI/PLGA particle formulations have the potential to treat pain and inflammation associated with acute gouty arthritis, an effect that may prove beneficial to patients.

### Example 23

### Intraarticular Sustained Release TKI Particles are Well Tolerated and Reduces Inflammation, Tissue Damage and Pain Associated with Chronic or Recurrent CPPD Arthropathy (Pseudogout Arthritis)

Patients with CPPD arthropathy are randomized to receive intraarticular TKI/PLGA particle formulations (low, intermediate and high dose) OR placebo in a double-blind, four parallel arm, dose-finding study, to evaluate the efficacy of TKI/PLGA particles. Inclusion criteria are definite CPPD disease, with prior or current calcium pyrophosphate crystals in fluid obtained from the acutely affected joint. Approximately 80 male or female patients 20-80 years old, who have acute CPPD of a specific joint are enrolled. Each arm includes a single intraarticular injection of TKI/PLGA particle formulations (low, intermediate and high dose) OR placebo into the joint with acute CPPD arthritis. The randomization ratio will be 1:1:1:1. The primary efficacy variable is improvement in pain relief, erythema, tenderness, swelling and inflammation in the affected acute CPPD joint at baseline as compared to the following time points: 1 day, 3 days, 5 days, 7 days, 10 days, 14 days, 21 days, and 28 days post-TKI/PLGA particle treatment. The reductions in joint pain, erythema, swelling and inflammation are consistently larger for the TKI/PLGA particle treated joints as compared to placebo treated joints.

Thus, we demonstrate that the TKI/PLGA particle formulations have the potential to treat pain and inflammation associated with acute CPPD arthritis, an effect that may prove beneficial to patients.

### Example 24

### Treatment of Allergic Rhinitis with Intra-Nasally-Administered TKI PLGA Particles

1) A 35-year-old woman has a history of nasal congestion on most days of the year, dating back to her late teens. She has chronic nasal drainage, which is clear and thick. Her congestion is worst in the late summer and early fall and again in the early spring; at these times, she also has sneezing, nasal itching, and cough. Five years ago, she had an episode of shortness of breath with wheezing on a day when her nasal symptoms were severe, but this episode resolved spontaneously and has not recurred. Her eyes do not bother her. Over-the-counter oral antihistamines help her symptoms a little but make her somnolent, nasal decongestants help but cause worsening symptoms after a couple days of use. She has not found intranasal corticosteroids or immunotherapy very helpful. Serum IgE testing is shows elevated IgE for dust mites. Patient is prescribed TKI/polymer particle intranasal administration and has follow-up evaluation for her allergic rhinitis symptoms.

2) A busy 28-year-old professional consults his physician for advice on long-standing hay fever. He reports having itchy eyes and an itchy nose, lacrimation, sneezing, rhinorrhea, and nasal congestion during the summer months. In previous years, he tried various antihistamines and nasal sprays, but these treatments only had limited benefit. A friend has suggested a corticosteroid injection or allergy injections, but he is hesitant to receive corticosteroids and unable to give up the time from work to receive allergy injections. An allergist evaluates him. Skin testing confirms that he is strongly sensitized to grass, pollen, and oak tree. A trial of sublingual immunotherapy is recommended but is hesitant given side effect profile and current FDA approval only for grass allergy. Patient is prescribed TKI/polymer particle intranasal administration and has follow-up evaluation for her allergic rhinitis symptoms.

### Example 25

### Intranasal TKI/Polymer Particles are Well Tolerated and Reduce Nasal Inflammation, Tissue Damage and Nasal Symptoms of Allergic Rhinitis

A study is conducted to show that TKI/polymer particles, as compared to placebo, can provide improvement in nasal symptoms of seasonal allergic rhinitis. This study is designed as a double-blind, randomized, placebo-controlled, four parallel arm, efficacy study of intranasal injections of TKI/polymer particle formulations in patients with allergic rhinitis. Each arm includes twice-weekly intranasal administration of one of three dosages of TKI/PLGA particle formulations (low, intermediate and high dose) OR placebo. The randomization ratio will be 1:1:1:1. This will be an outpatient study in adult men or women who have seasonal allergic rhinitis and have 2-year history (or longer) of mild to moderate allergic reaction to pollen/grass/trees/dust mite/animal or other allergen triggers. Qualified patients will be admitted to the single-blind 7-day Run-in Period (placebo daily) to establish the baseline allergic rhinitis symptom scores. Patient eligibility to enter the double-blind treatment phase will be based on patients' baseline nasal symptom scores. Eligible patients whose daytime average nasal symptom scores (of nasal congestion, nasal itching, rhinorrhea, and sneezing) is 2 or greater, with the daytime nasal congestion symptom score 2 or greater, on at least 4 of the 7 Run-in days will be admitted to the double-blind treatment phase, and randomized to either of three intranasal TKI/polymer particle doses (twice per week) or placebo treatment group. The primary endpoints are the Total Nasal Symptom Score (TNSS) which is the sum of scores for each of nasal congestion, sneezing, nasal itching, and rhinorrhea at each time point, using a four point scale (0-3), where 0 indicates no symptoms, a score of 1 for mild symptoms that are easily tolerated, 2 for awareness of symptoms which are bothersome but tolerable and 3 is reserved for severe symptoms that are hard to tolerate and interfere with daily activity. TNSS is calculated by adding the score for each of the symptoms to a total out of 12. Another method that will be used is the, the Visual Analogue Scale (VAS), a 10 cm scale that ranges from “no symptoms” to “worst symptoms ever” for each of the nasal symptoms. Secondary outcomes will be the Peak Nasal Inspiratory Flow (PNIF) for assessing nasal patency. PNIF provides an objective measurement of nasal airflow obstruction. It has the advantage of being simple, noninvasive and easily taught so participants can perform it on their own. Other secondary endpoints will be the Rhinoconjunctivitis Quality of Life Questionnaire (RQLQ) and monthly MD examination with nasal otoscopic evaluation for inflammation in visible mucosa and turbinates.

The percentage of patients with an improvement in their TNSS score, as measured at treatment time points is statistically larger for the TKI/PLGA particle formulations as compared to the placebo. The specified secondary endpoints also are statistically improved for the TKI/PLGA particle treated group. The results show TKI/polymer particle are able to slow, halt, treat and reverse nasal symptoms as well as nasal inflammation and tissue damage associated with allergic rhinitis as assessed based on MD exam, patient report of overall symptoms, TNSS, VAS, RQLQ.

### Example 26

### Treatment of Chronic Rhinosinusitis with TKI/PLGA Particles Delivered Intra-Nasally

1) A 42 year old male presents with nasal congestion, clear, thick nasal discharge, facial pain and pressure for 6 months. He denies recent illnesses. Skin prick testing is reviewed and is negative. He denies exacerbating factors and but notes persistent congestion and pain for past 6 months. A course of antibiotics prescribed by his doctor is not helpful. An ENT has ruled out anatomical pathology, he does not have nasal polyps. Allergy skin prick testing is negative. He has tried intranasal steroids and antihistamines without effect. Systemic decongestants work only temporarily and worsen his hypertension. Facial CT confirms sinonasal inflammation, with LMS score of 11. Patient is prescribed TKI/polymer particle intranasal injection and has follow-up evaluation for the severity of his rhinosinusitis symptoms.

2) A 73 year old male with a two to three decade history of recurrent nasal polyps associated with chronic sinusitis presented with complaints of nasal blockage, anosmia and disruption of sleep. Prior treatments include 2 surgeries for nasal polyposis and chronic sinusitis. Endoscopic exam revealed evidence of recurrent inflammatory polyps. A 4 week therapy of intranasal corticosteroids was not effective. The patient is prescribed TKI/polymer particles for intranasal use and has follow-up evaluation for the severity of his chronic sinusitis symptoms.

### Example 27

### Intranasal TKI/Polymer Particles are Well Tolerated and Reduce Nasal Inflammation, Tissue Damage, and Nasal Symptoms of Chronic Rhinosinusitis (CRS) with and without Nasal Polyps

A study is conducted to show that TKI/polymer particles, as compared to placebo, can provide improvement in nasal symptoms and inflammation of sinuses via imaging CRS with and without polyposis, which will be conducted in two separate trials. This study is designed as a double-blind, randomized, placebo-controlled, four parallel arm, efficacy study of intranasal administration of TKI/polymer particle formulations in patients with CRS+/−polyposis. Each arm includes a single intranasal administration of one of three dosages of TKI/polymer particle formulations (low, intermediate and high dose) OR placebo. The randomization ratio will be 1:1:1:1. Patients with confirmed diagnosis of chronic rhinosinusitis (CRS) by CT or MRI with a Lund-Makay-Score designation, with or without polyposis nasi grade I-III and PNIF of >7 l/min separated for left and right side of the nose are included in the study. During the Treatment Phase, patients will self-administer TKI/polymer or placebo per nostril every other day for 1, 2, 3, 4, 5, 6 days, through 14, 36, 52 weeks. Baseline serum IgE and skin prick testing is performed. A baseline chest X-ray is also performed. The primary endpoint is symptom specific measurements using one of three health related quality of life tools: 1) SNOT 22 (Sino-Nasal Outcome Test 22) score, a patient reported measure of outcome developed for use in CRS with or without nasal polyposis which covers a broad range of health and health-related quality of life problems including ‘physical problems, functional limitations and emotional consequences, as well as nasal blockage and changes in ability to smell; 2) Disability Index (RSDI) a validated, disease-specific quality-of-life survey designed for patients with sinonasal (sinus and nasal disease) disease. The RSDI has three separate subscales incorporating 30 questions with a total score range of 0-120; 3) The Chronic Sinusitis Survey (CSS) a validated, 6 question survey with two separate subscales which measure the impact of sinonasal symptoms and medication use in the preceding 8-week period. Total score range of 0-100 for total and subscale measures. Secondary endpoints are MD assessment during monthly visits, improvement of inflammation of the nasal mucosa and paranasal sinus as imaged by CT or MRI and Lund-Makay-Score (LMS). The LMS divides the sinus into six portion and the severity of sinus mucosal inflammation or fluid accumulation is scored as 0 (complete lucency), 1 (partial lucency) or 2 (complete opacity). Mild mucosal thickening without fluid collecting is scored as 0; mild mucosal thickening with fluid collecting causing partial lucency scored as 1; and, moderate or severe mucosal thickening without fluid collecting causing partial lucency, but not complete opacity, scored as 1. In addition, the ostiomeatal complex is scored as either 0 (not obstructed) or 2 (obstructed) because it is difficult to describe the ostiomeatal complex with any gradation (FIG. 1). The ten scores for the various sinuses and bilateral ostiomeatal complexes were summed to give a bilaterally total LMS that could range from 0 (complete lucency of all sinuses) to 24 (complete opacity of all sinuses). In addition, unilateral five portions of the sinuses from either the left or the right and one ipsilateral ostiomeatal complex were also summed to give separate unilaterally total LMS values that could range from 0 to 12, and finally change in size of polyps, polyposis nasi grade and recurrence of polyposis in patients who had a history of polyposis.

All treatments are well tolerated. The percentage of patients with an improvement in one of three HRQL scores (SNOT22, RSDI, and/or CSS), as measured at treatment time points is statistically larger for the TKI/polymer particle formulations as compared to the placebo. The specified secondary endpoints (including improved LMS scores) also support treatment with TKI/polymer particles. The percentage of patients with improvement in one of the HRQL scores compared to baseline level, as measured at the specified time points, is larger for the TKI/PLGA particle formulations as compared to placebo. The results will show TKI/polymer particles are able to slow, halt, treat and reverse nasal symptoms (through HRQL scales) as well as nasal inflammation associated with CRS (LMS scores) and in the case of CRS with polyposis, decrease polyp size, polypsis nasi grade, and prevent recurrence of polyps in those with history of polyps that have previously been removed.

### Example 28

### Treatment of Conjunctivitis by Ocular Administration of Ocular Drops Containing TKI/PLGA

1) A 20 year old male with history of atopy and seasonal allergies presents with rhinitis, sneezing and bilateral red, watery, itchy eyes. The ocular symptoms are most bothersome. He denies pain, foreign body sensation, vision changes. He notes similar symptoms every spring. Nasal antihistamines help temporarily, but he does not have improvement from saline or antihistamine eye drops. Allergy testing shows elevated IgE for grass, ragweed, and redwood trees. The patient is prescribed eye drops containing TKI/polymer particles which he administered topically every three days. The patient has follow up evaluation of his conjunctivitis symptoms.

### Example 29

### Ocular Administration of TKI/Polymer Particles are Well Tolerated and Reduce Inflammation, Tissue Damage and Symptoms of Allergic Conjunctivitis (AC)

A study is conducted to show that TKI/polymer particles, as compared to placebo is efficacious in providing improvement in ocular symptoms, including itching, and inflammation of eyes in allergic conjunctivitis. This study is designed as a double-blind, randomized, placebo-controlled, four parallel arm, efficacy study of intraocular administration of TKI/polymer particle formulations in patients allergic conjunctivitis. Each arm includes a single administration of one of three dosages of TKI/PLGA particle formulations (low, intermediate and high dose) OR placebo. The randomization ratio will be 1:1:1:1. Inclusion criteria include diagnostic skin test indicative of allergy for cat hair, cat dander, grasses, ragweed, dust mite, dog dander, cockroach and/or trees within 24 months prior to first visit, history of seasonal or perennial allergic conjunctivitis for at least 1 year prior to first visit, best-corrected visual acuity of 55 or greater in each eye as measured by ETDRS (letters read method), and manifest a positive bilateral Conjunctival Allergen Challenge (CAC) test response.

Enrolled participants will be tested for the presence of common allergens using the Conjunctival Allergen Challenge (CAC) model, which involves instillation of allergens directly into the eye to allow observations of acute allergic responses under controlled conditions. Drops of increasing concentration of a solubilized allergen will be instilled in both eyes until a positive reaction occurred. The test will be repeated to confirm the allergic reaction one week later. Participants with confirmed reactions will be administered the test article (Day 0) followed by treatment with TKI/polymer particles and be observed for 2 hours with changes measured over a matter of minutes, then again 24 hours after CAC-instillation (Day 1) and observed for 4 hours. The patient will return daily until day 7 to determine length of action of TKI/poylmer particles. Primary outcomes are mean ocular itching at onset of action and several time points <1 hr after administration and mean ocular itching at 24 hours duration of action. Secondary endpoints are mean total redness at onset and at 24 hours, mean conjunctival redness at onset and at 24 hours, proportion of responders to itching at onset and at 24 hours. Itching was assessed by the participant on a 0-4 scale (0=none, 4=incapacitating itch). Conjunctival redness was assessed by the investigator on a 0-4 scale (0=none, 4=extremely severe). Mean Total Redness at Onset of Action. Conjunctival redness, ciliary redness, and episcleral redness were assessed by the investigator on 0-4 scale (0=none, 4=extremely severe). Total redness is a composite variable summing conjunctival redness, ciliary redness, and episcleral redness scores (resultant score 0-12). All measurements were done for both eyes.

All treatments are well tolerated. The percentage of patients with an improvement in ocular itching at onset and at 24 hours consistently larger for the TKI/polymer particle formulations as compared to the placebo. The specified secondary endpoints also support TKI/polymer particle treatment as well as increased proportion of responders in those that received TKI/polymer particles. Unlike currently available therapies, which require daily or twice daily dosing, TKI/polymer particle efficacy is equal to or greater than one day. The results show TKI/polymer particle are able to slow, halt, treat and reverse ocular symptoms and inflammation related to AC based on symptoms and redness.

### Example 30

### Treatment of Uveitis with a TKI/PLGA Particle Delivered Topically to the Eye

1) A 38 year old male presented with right eye pain, with redness for 5 days associated with photophobia, excessive tearing, and reduced vision. On examination left eye was normal. On his right eye visual acuity was 6/12 with pin hole. He has circumcorneal congestion and had grade +4 cells and grade +3 flare in anterior chamber with posterior synechia and miosis. He doesn't have any clinical features suggestive of systemic disorder. He is diagnosed with uveitis, and prescribed eye drops containing TKI/polymer particles which he administered topically every three days. The patient has follow up evaluation of his conjunctivitis symptoms.

### Example 30

### Intraocular TKI/Polymer Particles are Well Tolerated and Reduce Inflammation, Tissue Damage and Symptoms of Uveitis

A study is conducted to show that TKI/polymer particles, as compared to placebo, is safe and efficacious in providing improvement in ocular symptoms, including pain, redness, and inflammation of eyes in uveitis. This study is designed as a double-blind, randomized, placebo-controlled, four parallel arm, dose-finding study and frequency of dose-finding study evaluating the safety, tolerability, and efficacy of intraocular administration of TKI/polymer particle formulations in patients with uveitis. Each arm includes a single administration of one of three dosages of TKI/PLGA particle formulations (low, intermediate and high dose) OR placebo. The randomization ratio will be 1:1:1:1. During the Treatment Phase, patients will self-administer TKI/polymer particle or placebo per eye daily for 1, 2, 3, 4, 5, 6 days, through 14, 36, 52 weeks. Inclusion criteria include active uveitis (Laser flare-cell meter score of at least 30 photons/ms) despite topical steroid therapy for least 1-3 months. The activity of uveitis will be evaluated by laser flare photometry, a recently validated technique for follow-up of the efficacy of treatments of uveitis. The primary endpoints are safety as evaluation of adverse events and a significant reduction of ocular inflammation after 2 months of treatment, quantified by laser flare photometry, considering the more severely affected eye in the case of bilateral uveitis. Clinical, laboratory and ophthalmological evaluation including laser flare photometry and conventional slit lamp examination will be performed at each visit (pre-inclusion, D0, D14, M1, M2, M3, M4, M5, M6, M9 and M12). Deterioration of ocular inflammation during the first 2 months will justify decoding for the patient concerned who will be considered to be a treatment failure. Secondary outcomes include patient perceived improvement and ability to wean off topical steroids.

All treatments are well tolerated. The primary endpoint, reduction of ocular inflammation after 2 months of treatment, quantified by laser flare photometry, compared to baseline is statistically larger for the TKI/polymer particle formulations administered as compared to the placebo. The specified secondary endpoints also support such a dose as the optimal dose and all show improvement in those that received TKI/polymer particles. The results show TKI/polymer particles are able to slow, halt, treat and reverse ocular symptoms and inflammation related to uveitis.

### Example 31

### Treatment of Eosinophilic Esophagitis (EOE) with TKI/PLGA Particles Administered Orally for Local Targeting of the Esophagus

1) 37 year old man presents with dysphagia for solid foods for 2 years. He complains of difficulty swallowing solid foods, and a history of food getting stuck in his throat and needing to vomit for clearance of impacted food. He notes heartburn, which is only intermittently improved with proton pump inhibitors. He denies food allergies. He is referred to GI and gets EGD which shows a stricture. Esophageal biopsy shows 17 eosinophils/hpf. He is resistant to start or budesonide for fear of esophageal candidiasis. He is started on TKI/polymer particle oral solution/suspension, and 4 months later is evaluated for symptoms of EOE.

### Example 32

### TKI/Polymer Particles are Well Tolerated and Reduce Inflammation, Tissue Damage and Symptoms of Eosinophilic Esophagitis (EOE)

A study is conducted to show that TKI/polymer particle, as compared to placebo, is safe and efficacious in providing improvement in symptoms and inflammation associated with EOE. This study is designed as a double-blind, randomized, placebo-controlled, four parallel arm, dose-finding study evaluating the efficacy of swallowed TKI/polymer particle formulations for local esophageal effect in patients with EOE. Each arm includes a single administration of one of three dosages of TKI/PLGA particle formulations (low, intermediate and high dose) OR placebo. The randomization ratio will be 1:1:1:1. During the Treatment Phase, patients swallow TKI/polymer particle or placebo every other day for 12 weeks. The Treatment Period will be 12 weeks during which subjects will visit the clinic at study weeks 0 (Baseline Visit), 2, 4, 8 and 12 (Final Treatment Evaluation) for clinical symptom assessment and safety evaluation (including adverse events and vital signs). Inclusion criteria include symptomatic adults based on the EoE Activity Index (EEsAI) PRO instrument (or other adult PRO) and with >15 eosinophils/HPF in 1 esophageal biopsy at baseline. The EEsAI is a validated scoring system that ranges from 0 to 100 points and includes seven items that assess frequency and duration of dysphagia episodes, severity of dysphagia caused by eating foods of eight different consistencies, and behavioral adaptations to living with dysphagia also assessed in the context of eating foods of eight different consistencies. Primary outcomes are percentage of improvement in PRO score and decreased eosinophil count on biopsy compared to baseline. Secondary outcomes included effects on esophageal remodeling after treatment, continued improvement of symptoms after completion of study treatment.

All treatments are well tolerated. The primary endpoint, percent of patients with patient symptomatic improvement and decrease in eosinophil count, compared to baseline is statistically larger for the TKI/polymer particle formulations as compared to the placebo, with one dose performing better than the others. The specified secondary endpoints are also met and all show improvement in those that received TKI/polymer particles. The results show TKI/polymer particles are able to slow, halt, treat and reverse the symptoms and inflammation associated with EOE.

### Example 33

### Treatment of Asthma with Inhaled TKI/PLGA Particles

1) A 29-year-old man with mild persistent asthma presents for a follow-up visit. He reports wheeze and cough 4 days a week and nocturnal symptoms three times a month. Spirometry reveals forced vital capacity (FVC) 85% predicted, forced expiratory volume in 1 second (FEV1) 75% predicted, FEV1/FVC 65%, and an increase in FEV1 of 220 ml or 14% following an inhaled short-acting bronchodilator. He is on a low-dose inhaled corticosteroid twice a day and a short-acting inhaled beta-agonist as needed. He also had symptoms of rhinitis; therefore he was referred to an allergist for evaluation. Skin testing is positive for trees, ragweed, dust mites, and cats. She is diagnosed with asthma, and prescribed Imatinib/PLGA to be inhaled twice per week. Three months later she has follow-up evaluation of her asthma symptoms.

2) A 40-year-old woman presents with wheeze and cough 3 days a week and nocturnal symptoms three times a month. Spirometry reveals forced vital capacity (FVC) 85% predicted, forced expiratory volume in 1 second (FEV1) 75% predicted, FEV1/FVC 65%, and an increase in FEV1 of 220 ml or 14% following an inhaled short-acting bronchodilator. She has no history of allergies. Skin testing is negative. She reports a viral respiratory infection 2 months ago after which symptoms started. She is diagnosed with post-infectious reactive airway disease, and is prescribed Imatinib/PLGA to be inhaled twice per week. Three months later she has follow-up evaluation of her asthma symptoms.

3) An 18 year old female college student presents to the student health center complaining of cough and chest tightness that occurs frequently with exercise. She is on the varsity field-hockey team and notes she occasionally has trouble keeping up with the other players during practice and games. Her coach has been criticizing her frequently for what he construes as “poor effort.” Her cough is episodic and is non-productive. Her dyspnea seems to occur after several minutes of exercise, and she states it feels like she cannot get a deep breath. She does not notice symptoms at other times of the day when she is not exercising. Her review of symptoms is otherwise unremarkable. She is diagnosed with exercise-induced asthma, and is prescribed Imatinib/PLGA to be inhaled twice per week. Three months later she has follow-up evaluation of her asthma symptoms.

4) A 33 year old African American male without significant past medical history, was transferred for evaluation of acute onset of dyspnea (<48 hours), wheezing and a cough. The patient denied a personal or family history of pulmonary disease. He was previously able to participate in athletic events without symptoms. He denied the use of tobacco, alcohol or drugs. He was employed as an industrial insulation application specialist. Approximately one day prior to presentation, he admitted to an unprotected exposure to a maleic anhydride gas cloud (used as a resin in fiberglass insulation). The patient denied any history of previous exposures. At the time of presentation, the patient did not have a fever or chills and did not report recent weight gain or lower extremity swelling. He had no chest pain, but did complain of chest tightness. He denied nausea, vomiting, diarrhea or abdominal pain. The remainder of his review of systems was unremarkable. He is diagnosed with asthma, and is prescribed Imatinib/PLGA to be inhaled twice per week. Three months later he has follow-up evaluation of his asthma symptoms.

5) A 49 year old white male presents to pulmonary clinic for evaluation of wheezing and dyspnea. He has a history of asthma since childhood that has been well-controlled off medication until this past year. He reports daily symptoms and almost nightly nocturnal awakenings due to shortness of breath, which is temporarily relieved with bronchodilators. He has had several exacerbations in the past 6 months and required hospitalization for an episode 1 month ago. He has been treated with tapering doses of oral prednisone for each exacerbation and reports his symptoms worsen each time he completes a steroid taper. His past medical history is also significant for perennial allergies and chronic sinusitis requiring three surgeries. His current medications include: fluticasone/salmeterol 500 μg/50 μg twice daily, zileuton 1200 mg twice daily, prednisone 10 mg daily, montelukast 10 mg once daily and albuterol on an as-needed basis which he is currently using four times daily. He was started on omalizumab 300 mg/month, 4 months ago without significant improvement. He is a lifelong nonsmoker and denies any illicit drug use. Sputum samples show increased eosinophils. He is diagnosed with asthma, and is prescribed Imatinib/PLGA to be inhaled twice per week. Three months later he has follow-up evaluation of his asthma symptoms.

### Example 35

### Inhaled TKI/Polymer Particles are Well Tolerated and Reduce the Inflammation, Tissue Damage, and Symptoms of Asthma

A study is conducted to show that TKI/polymer particle, as compared to placebo, is efficacious in providing improvement in symptoms and inflammation associated with asthma. This study is designed as a double-blind, randomized, placebo-controlled, four parallel arm, dose-finding study evaluating the efficacy of inhaled TKI/polymer particle formulations for several forms of asthma, including but not limited to allergic, non-allergic, exercise, occupational/environmental, severe/refractory, and eosinophilic/neutrophilic asthma. Each arm includes administration of one of three dosages of TKI/PLGA particle formulations (low, intermediate and high dose) OR placebo to each asthma cohort mentioned above. The randomization ratio will be 1:1:1:1. During the Treatment Phase, patients will inhale TKI/polymer particle or placebo every other day for 12 weeks. Eligible patients who have clinically diagnosed asthma requiring treatment with combined inhaled corticosteroid and long acting beta agonist according to Global Initiative for Asthma (GINA) guideline. The primary outcome is peripheral airway function measured by airway resistance at 5 and 20 Hz frequency from impulse oscillometry. The secondary outcomes are peak expiratory flow rate, forced expiratory flow at 1 second, forced vital capacity, forced expiratory flow at 25-75% of vital capacity (FEF25-75%) measured by spirometry, residual volume per total lung capacity ratio measured by body plethysmography, asthma control test score and asthma control questionaire-7 version, and sputum eosinophil count. All outcomes are measured at baseline and 2, 4, 6, and 12 weeks post treatments in all arms and across all cohorts. A baseline chest X-ray is also performed. Sputum samples are collected and analyzed at all visits.

All treatments are well tolerated. The primary endpoint, percent of patients with improvement of peripheral airway function measured by airway resistance, compared to baseline is statistically larger for the TKI/polymer particle formulations as compared to the placebo. The specified secondary endpoints are also improved in TKI/polymer particle recipients over placebo. The results show TKI/polymer particles are able to slow, halt, treat and reverse the symptoms and inflammation associated with asthma as seen by air resistance, symptoms, and measurement of inflammatory markers in sputum (including eosinophils).

### Example 36

### Treatment of Chronic Obstructive Pulmonary Disease (COPD) with Inhaled Imatinib/PLGA Particles

1) A 68 year-old male presents with worsening shortness of breath. He states feeling ‘out of breath’ and wheezing more. He cannot walk further than 5m, from his chair to the toilet. He has a worsening productive cough and has been producing yellow/green sputum. Past medical history is significant for COPD and 30-pack year smoking history. Recent spirometry results show forced FEV1: 55%, FEV1/FVC: 65%, of predicted. His current medications are albuterol as needed, fluticasone/salmeterol twice daily, however he continues to have frequent COPD exacerbations similar to the current presentation. He is prescribed Imatinib/PLGA to be inhaled twice per week. One month later he has follow-up evaluation of his COPD symptoms.

2) A 80-year-old woman who is a chronic smoker presents with a persistent cough for the last 4 months productive of white sputum. She had the same symptoms last year. Spirometry reveals forced expiratory volume in 1 second (FEV1) 60% predicted, FEV1/FVC 63%. She has no history of allergies. She is diagnosed with chronic bronchitis (a form of COPD), and is prescribed Imatinib/PLGA to be inhaled twice per week. Three months later she has follow-up evaluation of her chronic bronchitis symptoms.

### Example 37

### Inhaled Imatinib/Polymer Particles are Well Tolerated and Reduce the Inflammation, Tissue Damage, and Symptoms of COPD

A study is conducted to show that Imatinib/polymer particles, as compared to placebo, is efficacious in providing improvement in symptoms and inflammation associated with asthma. This study is designed as a double-blind, randomized, placebo-controlled, four parallel arm, dose-finding study evaluating the efficacy of inhaled Imatinib/PLGA particle formulations for COPD. Each arm includes a single administration of one of three dosages of TKI/PLGA particle formulations (low, intermediate and high dose) OR placebo. The randomization ratio will be 1:1:1:1. During the Treatment Phase, patients will inhale Imatinib/PLGA particle or placebo every other day for 12 months. Eligible patients have clinically diagnosed GOLD Stage I-III COPD, with or without smoking history. Patients will have site visits as months 1, 3, 6, 9, and 12. Primary endpoints are change in FEV1 and FEV1/FVC ratio and change in GOLD Stage group at 12 months. Secondary outcomes are change in GOLD stage and spirometry (both FEV and FEV1/FVC) as above at time points 1, 3, 6, 9 months, quality of life per CAT and CCQ scores, symptom scores by MMRC dyspnea scale, time to first COPD exacerbation and number of COPD exacerbations. A baseline chest X-ray is also performed.

The primary endpoints of change in FEV1 and FEV1/FVC ratio and change in GOLD Stage group at 12 months, compared to baseline is consistently significantly improved for the Imatinib/PLGA particle formulations as compared to the placebo. The specified secondary endpoints are improved in Imatinib/PLGA particle recipients over placebo. The results show Imatinib/PLGA particles are able to slow, halt, treat and reverse the symptoms and inflammation associated with COPD as seen by change in FEV1 and FEV1/FVC ratio and change in GOLD Stage, quality of life per CAT and CCQ scores, symptom scores by MMRC dyspnea scale, time to first COPD exacerbation and number of COPD exacerbations.

### Example 38

### Treatment of Aspirin-Exacerbated Respiratory Disease (AERD) with TKI/PLGA Particles Administered Intranasally

1) A 43 year old African American female presented after a reaction to aspirin. She had a history of severe asthma and was using twice daily inhaled fluticasone propionate/salmeterol in a 500/50 microgram combination formulation, montelukast 10 mg/day, albuterol metered dose inhaler and tiotropium 18 mcg inhaled per day. She had been on multiple courses of systemic prednisone but not in the prior month. She had a history of 3 prior sinus operations which included polypectomies. She also has chronic sinusitis confirmed by CT scan. She reported having been intubated after taking ibuprofen in the past, which she developed respiratory distress almost immediately after ingestion. She endorses allergies to penicillin, egg and shrimp. She is started on intranasal TKI/polymer particle for a period of time and then undergoes aspirin desensitization with concomitant use of intranasal TKI/polymer.

2) A 43 year old African American female presented after a reaction to aspirin. She had a history of severe asthma and was using twice daily inhaled fluticasone propionate/salmeterol in a 500/50 microgram combination formulation, montelukast 10 mg/day, albuterol metered dose inhaler and tiotropium 18 mcg inhaled per day. She had been on multiple courses of systemic prednisone but not in the prior month. She had a history of 3 prior sinus operations which included polypectomies. She also has chronic sinusitis confirmed by CT scan. She reported having been intubated after taking ibuprofen in the past, which she developed respiratory distress almost immediately after ingestion. She endorses allergies to penicillin, egg and shrimp. Sputum analysis shows increased eosinophils. She is started on intranasal TKI/polymer particle for a period of time prior to desensitization and then undergoes aspirin desensitization with concomitant use of intranasal TKI/polymer.

### Example 36

### Intranasal or Inhaled TKI/Polymer Particles are Well Tolerated and Reduce Inflammation, Tissue Damage and Symptoms (Including During Desensitization) of Aspirin-Exacerbated Respiratory Disease (AERD)

A study is conducted to show that TKI/polymer particle, as compared to placebo, is efficacious when inhaled or nasally administered prior to aspirin desensitization will reduce severity of aspirin-induced respiratory reaction, and improve associated conditions such as asthma and rhinosinusitis. This study is designed as a double-blind, randomized, placebo-controlled, four parallel arm, dose-finding study evaluating the efficacy of inhaled TKI/polymer particle formulations for AERD without and without increased sputum eosinophils. Each arm includes a single administration of one of three dosages of TKI/PLGA particle formulations (low, intermediate and high dose) OR placebo. The randomization ratio will be 1:1:1:1. Subjects must meet inclusion criteria including diagnostic criteria for AERD and be a candidate for aspirin desensitization. Subjects can also have chronic asthma and chronic rhinosinusitis. Sinusitis will have been confirmed by imaging studies presently and/or in the past. All patients must have a history of adverse reaction to aspirin and/or aspirin-like drugs (e.g., ibuprofen, naproxen, etc.) compatible with AERD. During the Treatment Phase, patients will inhale TKI/polymer particle or placebo every other day for 24 weeks. All outcomes are measured at baseline and 2, 4, 6, and 12 weeks post treatments as well as during aspirin desensitization. Aspirin desensitization will occur 1-4 weeks after starting therapy. Primary outcomes include percent of patients reacting during aspirin desensitization, and severity of reaction, including TNSS for subjects with AERD during the clinical reaction to aspirin challenge. Secondary endpoints will evaluate dose of aspirin causing reaction, dose of aspirin needed to maintain desensitization, change in associated conditions including asthma and rhinosinusitis. Another study will be conducted with intranasal administration of TKI/polymer particles to assess for improvement in AERD symptoms through this mode of administration.

The primary endpoint, percent of patients with improvement of reaction during aspirin desensitization compared to baseline is consistently larger for the TKI/polymer particle formulations as compared to the placebo. The specified secondary endpoints are also improved in TKI/polymer particle recipients over placebo. The results show TKI/polymer particles are able to slow, halt, treat and reverse the symptoms and inflammation associated with AERD as evaluated by symptoms and reactions during and after desensitization and measurement of inflammatory markers in sputum (including eosinophils).

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. Applicants reserve the right to pursue such inventions in later claims.

What is claimed is:

1. A method of treating a mast cell-mediated inflammatory disease, comprising:
locally administering a therapeutically effective amount of a tyrosine kinase inhibitor to a patient in need thereof.

2. The method of claim 1, wherein the mast cell-mediated inflammatory disease is a joint disease selected from the group consisting of osteoarthritis, gout, calcium pyrophosphate dihydrate deposition disease, hydroxyapatite crystal deposition disease, or calcific tendonitis.

3. The method of claim 1, wherein the mast cell-mediated inflammatory disease is a disease selected from the group consisting of allergic rhinitis, chronic rhinitis, chronic rhinosinusitis, chronic obstructive pulmonary disease (COPD), asthma, eosinophilic esophagitis, aspirin exacerbated respiratory disease (AERD), or uveitis.

4. The method of claim 1, wherein the tyrosine kinase inhibitor is selected from inhibitors targeting a member of the JAK, KIT, SYK, or SRC family of kinases.

5. The method of claim 1, wherein the tyrosine kinase inhibitor is selected from the group consisting of imatinib, dasatinib, tofacitinib, fostamitinib, ruxolitinib, nilotinib, baricitinib, or ponatinib.

6. A method of treating a mast cell-mediated inflammatory joint disease, comprising:
injecting a plurality of sustained release particles into a joint of a patient in need thereof, wherein:
said patient joint is affected by said inflammatory joint disease, and
the sustained release particles comprise a therapeutically effective amount of a tyrosine kinase inhibitor.

7. A method of treating a mast cell-mediated inflammatory disease, comprising:
local administration of a plurality of sustained release particles to a patient in need thereof, wherein:
said patient is affected by said mast cell-mediated inflammatory disease, and the sustained release particles comprise a therapeutically effective amount of a tyrosine kinase inhibitor.

8. The method of claim 6, wherein said sustained release particles comprise a biodegradable polymer and the tyrosine kinase inhibitor.

9. The method of claim 8, wherein the biodegradable polymer is selected from the group consisting of PLGA polymers.

10. The method of claim 6, wherein the tyrosine kinase inhibitor is selected from the group consisting of imatinib, dasatinib, fostamatinib, tofacitinib ruxolitinib, nilotinib, baricitinib, and ponatinib.

11. A pharmaceutical composition comprising a plurality of sustained release particles comprising a biodegradable polymer and a tyrosine kinase inhibitor, wherein said sustained release particles have a biomodal particle size distribution which provides 10% TKI release per week and provide therapeutically effective levels of TKI for 2 months.

12. The method of any of claim 2, wherein said administering comprises injecting a plurality of sustained release particles comprising a therapeutically effective amount of a tyrosine kinase inhibitor into a joint affected by the joint disease of a patient in need thereof.

13. The method of any of claim 3, comprising local administration of a plurality of sustained release particles comprising a therapeutically effective amount of a tyrosine kinase inhibitor into the eye, sinuses, esophagus, or lungs of a patient in need thereof.

14. The composition of claim 11, wherein the biodegradable polymer is selected from the group consisting of polymers of D-lactic acid, L-lactic acid, racemic lactic acid, glycolic acid, polycaprolactone, or combinations thereof.

15. The method of claim 8, wherein the biodegradable polymer is selected from the group consisting of polymers of D-lactic acid, L-lactic acid, racemic lactic acid, glycolic acid, polycaprolactone, or combinations thereof.

16. The composition of claim 11, wherein the biodegradable polymer is polylactic-co-glycolic acid (PLGA).

17. The method of claim 8, wherein the biodegradable polymer is polylactic-co-glycolic acid (PLGA).

18. The composition of claim 11, wherein the tyrosine kinase inhibitor is selected from the group consisting of imatinib, dasatinib, tofacitinib, fostamitinib, ruxolitinib, nilotinib, baricitinib, and ponatinib.
